(19) United States
(12) Patent Application Publication
(10) Pub. No.: US 2023/0279018 A1
(43) Pub. Date: Sep. 7, 2023

(54) APICOMPLEXAN PARASITE INHIBITION

(71) Applicants: The Broad Institute, Inc., Cambridge, MA (US); President and Fellows of Harvard College, Cambridge, MA (US); Eisai R&D Management Co., LTD., Tokyo, Chiba (JP)

(72) Inventors: Eamon COMER, Cambridge, MA (US); Bruno MELILLO, Cambridge, MA (US); Francis G. FANG, Andover, MA (US); Branko MITASEV, North Reading, MA (US)

(73) Assignees: The Broad Institute, Inc., Cambridge, MA (US); President and Fellows of Harvard College, Cambridge, MA (US); Eisai R&D Management Co., LTD., Tokyo, Chiba (JP)

(21) Appl. No.: 18/188,249

(22) Filed: Mar. 22, 2023

Related U.S. Application Data
(63) Continuation of application No. PCT/US2021/051986, filed on Sep. 24, 2021.
(60) Provisional application No. 63/083,028, filed on Sep. 24, 2020.
(60) Provisional application No. 63/083,065, filed on Sep. 24, 2020.

(51) Int. Cl. C07D 487/04; A61P 33/06; A61P 33/02

(52) U.S. Cl. CPC C07D 487/04; A61P 33/06; A61P 33/02

(57) ABSTRACT

The invention provides agents that inhibit L-Phe in cytoplasmic phenylalanine tRNA-synthetase (cFRS), methods for identifying them, compositions comprising such agents, and therapeutic methods of using such agents.

### CROSS REFERENCE TO RELATED APPLICATIONS

This application is a U.S. utility application under 35 U.S.C. 111(a) that is a continuation of PCT International Patent Application No. PCT/US2021/051986, filed Sep. 24, 2021, designating the United States and published in English, which claims priority to and the benefit of U.S. Provisional Patent Application No. 63/083,028, filed Sep. 24, 2020, and U.S. Provisional Patent Application No. 63/083,065, filed Sep. 24, 2020, each of which are hereby incorporated by reference in their entirety.

### SEQUENCE LISTING

The present application contains a Sequence Listing which has been submitted electronically in XML format following conversion from the originally filed TXT format.

The content of the electronic XML Sequence Listing, (Date of creation: Mar. 22, 2023; Size: 45,018 bytes; Name: 167741-025002US-Sequence_Listing.xml), and the original TXT format, is herein incorporated by reference in its entirety.

### BACKGROUND OF THE INVENTION

Species in the phylum Apicomplexa are parasitic alveolates that result in a wide array of diseases upon host infection. Apicomplexan species often also develop strains resistant to active agents. For example, malaria, caused by apicomplexan parasites from the genus Plasmodium, presents a formidable global health challenge mainly due to the emergence of parasite strains that are resistant to front-line drugs. It is therefore necessary to discover and validate new drug targets, as well as compounds whose efficacy is unaffected by mechanisms of resistance to traditional antiparasitic agents. Ideally, such candidates should have a new mechanism of action (MoA) with rapid asexual blood-stage parasite reduction and activity against all stages of the parasite lifecycle in the human host.

### SUMMARY OF THE INVENTION

In accordance with the foregoing objectives and others, the present disclosure provides compounds with unique MoA for inhibition of the FRS enzyme in apicomplexan parasites, methods for identifying such compounds, and therapeutic methods for their use in treating disease (e.g., malaria, toxoplasmosis, and cryptosporidiosis). Furthermore, these compounds do not inhibit or do not significantly inhibit a human ortholog of FRS.

In one aspect, the compound has the structure of formula (I):

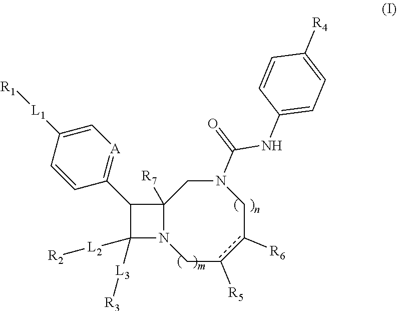

wherein the dashed bond (———) may be a single or double bond;
m is 0 (i.e., it is a bond) or 1;
n is 0, 1 or 2;
A is CH or N;
L₁ is absent, or —C≡C—;
L₂ and L₃ are independently absent, alkylene (e.g., C₁-C₄ alkylene, methylene), or heteroalkylene (e.g., C₁-C₄ heteroalkylene), wherein any of the foregoing groups optionally comprises one or more (e.g., one, two, three) points of substitution;
R₁ is hydrogen, aryl (e.g., C₅-C₁₂ aryl), or heteroaryl (e.g., C₅-C₁₂ heteroaryl), wherein any of the foregoing groups optionally comprises one or more (e.g., one, two, three) points of substitution;
R₂ and R₃ are independently hydrogen, —OH, —OR, —S(O)₂R, —N(R)S(O)₂R, —C(O)R, —OC(O)R, —N(R)C(O)R, —C(O)N(R)R, —N(R)₂, or heterocyclyl, and R₂ and/or R₃ has one or more (e.g., two, three, four, five) optional points of substitution;
R₄ is cycloalkoxy (e.g., C₃-C₆ cycloalkoxy, cyclopropoxy) optionally comprising one or more (e.g., one, two, three) points of substitution;
R₅ and R₆ are independently selected from hydrogen and —OH;
R₇ is hydrogen, —CH₂OH, or —CH₂OR;
R is independently selected at each occurrence from hydrogen and alkyl (e.g., C₁-C₁₂ alkyl, C₁-C₈ alkyl, C₁-C₅ alkyl, C₃-C₁₂ cycloalkyl), wherein each R has one or more (e.g., two, three, four, five) optional points of substitution (e.g., with —OH, with —C(O)OH, —CN, —NH₂, —N(R^{A})₂); and
R^{A} is independently selected at each occurrence from hydrogen and lower alkyl (e.g., C₁-C₄ alkyl, methyl, ethyl, propyl, isopropyl); or
pharmaceutically acceptable salts thereof; or
prodrugs of any of the foregoing;
wherein said compound is not

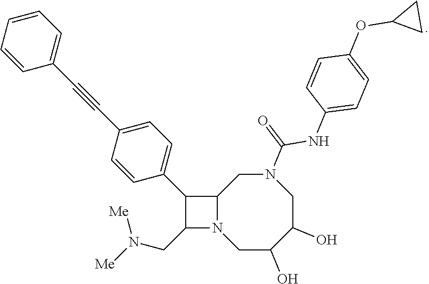 [SMILES: CN(C)CC1C(c2ccc(C#Cc3ccccc3)cc2)C2CN(C(=O)Nc3ccc(OC4CC4)cc3)CC(O)C(O)CN12]

In one embodiment, the compound has the structure of formula (II):

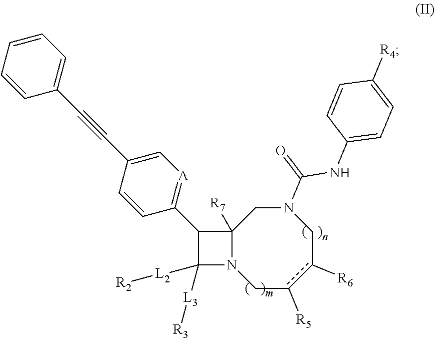

pharmaceutically acceptable salts thereof; or
prodrugs of any of the foregoing.

In another embodiment, the compound has the structure of formula (III):

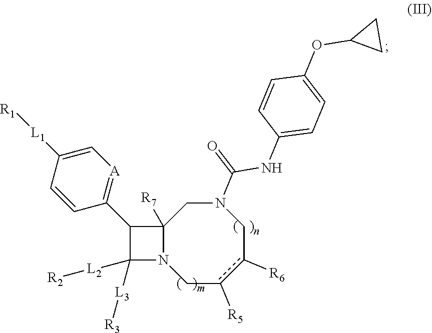

pharmaceutically acceptable salts thereof; or
prodrugs of any of the foregoing.

In yet another embodiment, the compound has the structure:

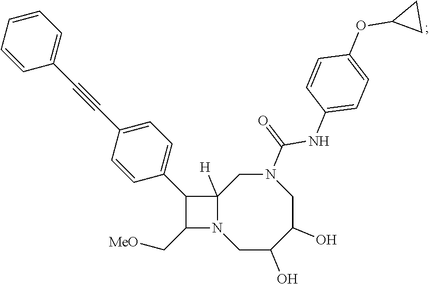 [SMILES: COCC1C(c2ccc(C#Cc3ccccc3)cc2)C2CN(C(=O)Nc3ccc(OC4CC4)cc3)CC(O)C(O)CN12]

or including enantiomers, diastereomers, or mixtures of enantiomers and/or diastereomers (e.g., racemic mixture) thereof,
or a pharmaceutically acceptable salt and/or prodrug of any of the foregoing. For example, in some embodiments, the compound has the structure of:

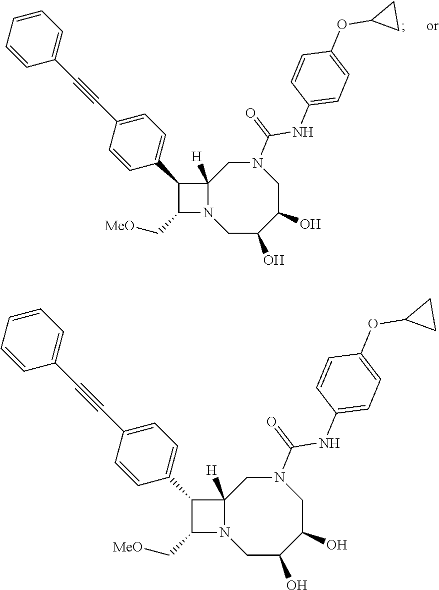 [SMILES: COC[C@@H]1[C@@H](c2ccc(C#Cc3ccccc3)cc2)[C@@H]2CN(C(=O)Nc3ccc(OC4CC4)cc3)C[C@@H](O)[C@@H](O)CN12 ; COC[C@@H]1[C@H](c2ccc(C#Cc3ccccc3)cc2)[C@@H]2CN(C(=O)Nc3ccc(OC4CC4)cc3)C[C@@H](O)[C@@H](O)CN12]

or is a racemic mixture thereof. In some embodiments, the compound as the structure of Compound 1:

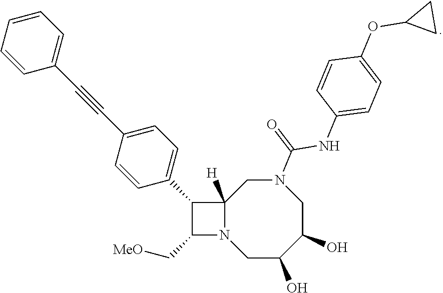 [SMILES: COC[C@@H]1[C@H](c2ccc(C#Cc3ccccc3)cc2)[C@@H]2CN(C(=O)Nc3ccc(OC4CC4)cc3)C[C@@H](O)[C@@H](O)CN12]

In another aspect, the invention provides a pharmaceutical composition containing a pharmaceutically acceptable excipient and the compound according to any previous aspect, or a pharmaceutical salt thereof, or a prodrug of any of the foregoing.

In another aspect, the invention provides a crystalline form of cytoplasmic phenylalanyl-tRNA synthetase enzyme from an apicomplexan species bound to a compound having the structure of any previous aspect.

In one embodiment, the enzyme is cytoplasmic phenylalanyl-tRNA synthetase enzyme from a species in the Plasmodium genus (PcFRS).

In another embodiment, the enzyme is Plasmodium falciparum: cytoplasmic phenylalanyl-tRNA synthetase (PfcFRS).

In yet another embodiment, the enzyme is Plasmodium vivax cytoplasmic phenylalanyl-tRNA synthetase (PvcFRS).

In still another embodiment, the crystalline form is characterized by the Protein Data Bank Structure ID 7BY6, which is hereby incorporated by reference in its entirety corresponding to the protein atomic coordinates in the three-dimensional protein crystal structure in Table 1 of U.S. App. No. 63/083,028, which is hereby incorporated by reference in its entirety and particularly, Table 1. Protein Data Bank Structure ID 7BY6 also includes the structure and orientation of Compound 1 when crystallized with the protein derived from apicomplexan parasite phenylalanyl-tRNA synthetase.

In still another embodiment, the crystalline form diffracts to a resolution of from 2 to 4 Å (e.g., from 2.5 to 3.5 Å, from 3.9 to 4.1 Å, and all values in between).

The unique mechanisms of action for these inhibitors were in part elucidated following the preparation of crystalline forms of apicomplexan phenylalanyl t-RNA synthetase complex with bicyclic azetedines. For example, crystalline forms of apicomplexan FRS polypeptides with drug candidate compounds are provided herein. In some embodiments, the crystalline form is complexed with a compound having the structure of Formula (IV):

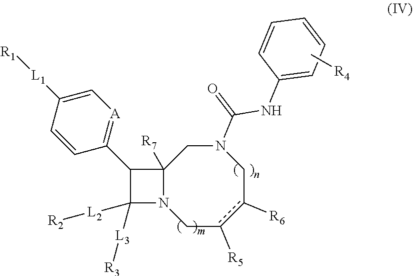

wherein the dashed bond (———) may be a single or double bond;
m is 0 (i.e., it is a bond) or 1;
n is 0, 1 or 2;
A is CH or N;
L₁ is absent, or —C≡C—;
L₂ and L₃ are independently absent, alkylene (e.g., C₁-C₄ alkylene, methylene), or heteroalkylene (e.g., C₁-C₄ heteroalkylene), wherein any of the foregoing groups optionally comprises one or more (e.g., one, two, three) points of substitution;
R₁ is hydrogen, alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, C₁-C₈ heteroalkyl, C₁-C₅ heteroalkyl, C₃-C₁₂ heterocycloalkyl), halogen (e.g., fluoro, chloro), aryl (e.g., C₆-C₁₂ aryl, phenyl), or heteroaryl (e.g., C₅-C₁₂ heteroaryl, pyridinyl), and R₁ has one or more (e.g., two, three, four, five) optional points of substitution;
R₂ and R₃ are independently hydrogen, —OH, —OR, —S(O)₂R, —N(R)S(O)₂R, —C(O)R, —OC(O)R, —N(R)C(O)R, —C(O)N(R)R, —N(R)₂, or heterocyclyl, and R₂ and/or R₃ has one or more (e.g., two, three, four, five) optional points of substitution;
R₄ is hydrogen, perfluoroalkyl, aryl (e.g., C₆-C₁₂ aryl, phenyl), arylalkyl (e.g., C₇-C₁₄ alkylaryl, benzyl), alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, alkoxy such as C₁-C₁₂ alkoxy or C₃-C₈ cycloalkoxy), or heteroaryl (e.g., C₅-C₁₂ heteroaryl, pyridinyl), and R₄ has one or more (e.g., two, three, four, five) optional points of substitution (e.g., with alkoxy, fluoroalkoxy);
R₅ and R₆ are independently selected from hydrogen and —OH;
R₇ is hydrogen, —CH₂OH, or —CH₂OR;
R is independently selected at each occurrence from hydrogen and alkyl (e.g., C₁-C₁₂ alkyl, C₁-C₈ alkyl, C₁-C₅ alkyl, C₃-C₁₂ cycloalkyl), wherein each R has one or more (e.g., two, three, four, five) optional points of substitution (e.g., with —OH, with C(O)OH, —CN, —NH₂, —N(R^{A})₂); and
R^{A} is independently selected at each occurrence from hydrogen and lower alkyl (e.g., C₁-C₄ alkyl, methyl, ethyl, propyl, isopropyl); or
pharmaceutically acceptable salts thereof; or
prodrugs of any of the foregoing.

In another aspect, the invention provides a method for identifying an agent that binds to a binding pocket of a cytoplasmic phenylalanyl-tRNA synthetase enzyme from an apicomplexan species or a fragment thereof are also provided which may comprise:
(a) performing a computational fitting operation between a candidate agent (e.g., a compound having the structure of Formula (IV) or Formula (I)) and a three-dimensional representation of the apicomplexan enzyme; and
(b) quantifying an association between the candidate agent and the three-dimensional representation of the L-Phe binding site of the apicomplexan enzyme and/or the ATP binding site and/or the Auxiliary binding site of the apicomplexan enzyme, thereby identifying an agent that binds to a binding pocket of the apicomplexan enzyme or a fragment thereof.

The present disclosure includes methods for identifying an agent that binds to a binding pocket of a cytoplasmic phenylalanyl-tRNA synthetase enzyme from an apicomplexan species or a fragment thereof which comprises:
(a) generating a three-dimensional representation of a binding pocket of apicomplexan enzyme or a fragment thereof from an X-ray crystal structure of one or more binding pockets of the apicomplexan enzyme or a fragment thereof,
(b) identifying the amino acids of the binding pocket of apicomplexan enzyme or a fragment thereof;
(c) generating a three-dimensional representation of the apicomplexan enzyme or a fragment thereof;
(d) performing a computational fitting operation between a candidate agent (e.g., a compound having the structure of Formula (IV) or Formula (I)) and the three-dimensional representation of the apicomplexan vivax enzyme or a fragment thereof, and
(e) quantifying an association between the candidate agent and the three-dimensional representation of the apicomplexan enzyme or a fragment thereof, thereby identifying an agent that binds to apicomplexan enzyme or a fragment thereof.

Methods of use of these inhibitors are also provided. For example, a method of treatment or prophylaxis of a parasitic disease caused by an apicomplexan parasite in a subject in need thereof comprises administration to said subject the compound of the present disclosure (e.g., a compound of formula (I), a compound of formula (IV), a compound of identified in the screening assays described herein), or a pharmaceutical salt thereof, or a prodrug of any of the foregoing. For example, the compound is formulated in a pharmaceutical composition comprising the compound and one or more pharmaceutically acceptable carriers, excipients, and/or diluents.

Methods of inhibiting or preventing the growth of a population of apicomplexan parasites in a medium are also part of the present disclosure comprising contacting the population with a compound of the present disclosure (e.g., a compound of formula (I), a compound of formula (IV), a compound of identified in the screening assays described herein).

In various embodiments of the above aspects, the enzyme is cytoplasmic phenylalanyl-tRNA synthetase enzyme from a species in the Plasmodium genus (PcFRS). In other embodiments, the enzyme is Plasmodium falciparum: cytoplasmic phenylalanyl-tRNA synthetase (PfcFRS). In still other embodiments, the enzyme is Plasmodium vivax cytoplasmic phenylalanyl-tRNA synthetase (PvcFRS). In still other embodiments, the one or more binding pockets comprises one or more of an amino acid sequence selected from amino acids 443-552 of Plasmodium vivax enzyme. In still other embodiments, the Plasmodium vivax enzyme or a fragment thereof binding pocket comprises an amino acid selected from the group consisting of Arg443, Glu445, Val458, His451, Phe455, Gln457, Glu459, Tyr480, I1e483, Tyr 497, Gly506, His508, Glu510, Lys512, Lys513, Leu515, Val517, Asn519, Ala541, Trp542, Gly543, Leu544, Pro549, and I1e552. In still other embodiments, the agent is selected from a compound having the structure of formula (IV):

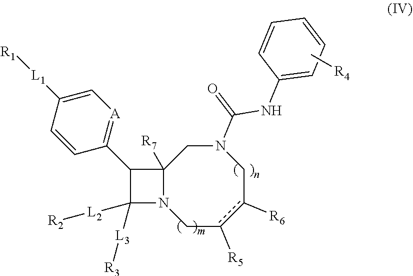

wherein the dashed bond (———) may be a single or double bond;
m is 0 (i.e., it is a bond) or 1;
n is 0, 1 or 2;
A is CH or N;
L₁ is absent, or —C≡C—;
L₂ and L₃ are independently absent, alkylene (e.g., C₁-C₄ alkylene, methylene), or heteroalkylene (e.g., C₁-C₄ heteroalkylene), wherein any of the foregoing groups optionally comprises one or more (e.g., one, two, three) points of substitution;
R₁ is hydrogen, alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, C₁-C₈ heteroalkyl, C₁-C₅ heteroalkyl, C₃-C₁₂ heterocycloalkyl), halogen (e.g., fluoro, chloro), aryl (e.g., C₆-C₁₂ aryl, phenyl), or heteroaryl (e.g., C₅-C₁₂ heteroaryl, pyridinyl), and R₁ has one or more (e.g., two, three, four, five) optional points of substitution;
R₂ and R₃ are independently hydrogen, —OH, —OR, —S(O)₂R, —N(R)S(O)₂R, —C(O)R, —OC(O)R, —N(R)C(O)R, —C(O)N(R)R, —N(R)₂, or heterocyclyl, and R₂ and/or R₃ has one or more (e.g., two, three, four, five) optional points of substitution;
R₄ is hydrogen, perfluoroalkyl, aryl (e.g., C₆-C₁₂ aryl, phenyl), arylalkyl (e.g., C₇-C₁₄ alkylaryl, benzyl), alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, alkoxy such as C₁-C₁₂ alkoxy or C₃-C₈ cycloalkoxy), or heteroaryl (e.g., C₅-C₁₂ heteroaryl, pyridinyl), and R₄ has one or more (e.g., two, three, four, five) optional points of substitution (e.g., with alkoxy, fluoroalkoxy);
R₅ and R₆ are independently selected from hydrogen and —OH;
R₇ is hydrogen, —CH₂OH, or —CH₂OR;
R is independently selected at each occurrence from hydrogen and alkyl (e.g., C₁-C₁₂ alkyl, C₁-C₈ alkyl, C₁-C₅ alkyl, C₃-C₁₂ cycloalkyl), wherein each R has one or more (e.g., two, three, four, five) optional points of substitution (e.g., with —OH, with C(O)OH, —CN, —NH₂, —N(R^{A})₂); and
R^{A} is independently selected at each occurrence from hydrogen and lower alkyl (e.g., C₁-C₄ alkyl, methyl, ethyl, propyl, isopropyl); or
pharmaceutically acceptable salts thereof; or prodrugs of the foregoing.

In various embodiments of the above aspects, the apicomplexan parasite is Plasmodium (e.g., Plasmodium falciparum (Pf), Plasmodium vivax (Pv), Plasmodium ovale (Po), Plasmodium malariae (Pm), Plasmodium fragile (Pfr), Plasmodium inui (Pi), or Plasmodium gonderi (Pg)), Cryptosporidium (e.g., Cryptosporidium parvum (Cp) or Cryptosporidium hominis (Ch)) or Toxoplasma (e.g., Toxoplasma gondii (Tg)). In certain embodiments, the apicomplexan parasite is Plasmodium falciparum. In various aspects, the apicomplexan parasite is Plasmodium vivax. In various embodiments of the above aspects, the subject is a human.

### Definitions

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half-integral numeric values are also contemplated as specifically disclosed and as limits for all subsets of the disclosed range. For example, a range of from 0.10% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, or from 0.1% to 2.5%. It will be understood that the sum of all weight % of individual components will not exceed 100%.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented. Unless otherwise specified, the terms “first,” “second,” and the like, “primary,” “secondary,” and the like, do not denote any order, quantity, or importance, but rather are used to distinguish one element from another.

By “agent” is meant a small compound, polypeptide or polynucleotide.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

As used herein “apicomplexan phenylalanyl-tRNA synthetase polypeptide” or “apicomplexan FRS polypeptide” refers to polypeptides of the phenylalanyl-tRNA synthetase enzyme of an apicomplexan parasite. Apicomplexan FRS polypeptides include the cytoplasmic FRS polypeptides (cFRS). The apicomplexan FRS polypeptide may be from a species from the genus Plasmodium (e.g., Plasmodium falciparum (Pj), Plasmodium vivax (Pv), Plasmodium ovale (Po), Plasmodium malariae (Pm), Plasmodium fragile (Pfr), Plasmodium inui (Pi), Plasmodium gonderi (Pg)), Cryptosporidium (e.g., Cryptosporidium parvum (Cp), Cryptosporidium hominis (Ch)), or Toxoplasma (e.g., Toxoplasma gondii (Tg)). The apicomplexan polypeptide may include dimers of the alpha and beta subunits including heterodimeric forms which may optionally further dimerize to include heterotetramers (e.g., α2β2).

For example, the apicomplexan FRS polypeptide may be a protein or fragment thereof having at least 85% amino acid sequence identity to the Plasmodium fragile FRS a subunit sequence provided at NCBI Ref No. XP_012338036 or a fragment thereof that functions in protein synthesis. An exemplary Plasmodium fragile FRS a subunit sequence is provided below:

| | | | | | SEQ ID NO: 1 | |
|---|---|---|---|---|---|---|
| 1 | MQAKGQEEQK | GEELNHFMQV | LEEEFALCHD | MREKREVDDA | WLGNAEAEEL | ANHRAYYLQL |
| 61 | QEEKNVQVEE | TKYVTSLHMS | KKYNLEHIKV | LGMAKKLETL | YYVVNHVRSF | NTYQLTEEGK |
| 121 | EYLRDGSPEY | VTLKYVVEQQ | TCSLEDLKKL | FGKKGDIGLN | INLKKKKIEL | RKSDKRLCHH |
| 181 | VEGSANSLVD | ETRRHLDMVE | THGHDEGTLV | SHLKDIVTCG | KKDEEVNNLI | KDLKKRKLIE |
| 241 | VKKVSYMYIV | RTNHFTKEIK | KQITDLTYLL | IKNEEYKKYE | VKKYNFFSSG | KKMNKGNVHL |
| 301 | LIKQMRTFKE | VFISLGFEEM | DTHNYVESSF | WCFDALYIPQ | QHPSRDLQDT | FFIEAPELCK |
| 361 | DNFTDQSYID | NVKRVHSVGD | YGSFGWNYPW | ELKSTKKNVL | RTHTTANSCR | ALYQLAKEYQ |
| 421 | KTGSIIPKKY | FSIDRVFRNE | NLDSTHLAEF | HQVEGLIIDK | NLGLSHLIGT | LGAFYKYIGI |
| 481 | SKLRFKPTFN | PYTEPSMEVY | GYHEENKKWM | EVGNSGIFRP | EMLRAMGFPA | EVSVIAWGLS |
| 541 | LERPTMIKYN | IRNIRDLFGY | RSVL. | | | |

The apicomplexan FRS polypeptide may be a protein or fragment thereof having at least 85% amino acid sequence identity to the Plasmodium falciparum: FRS α subunit sequence or fragment thereof, such as the sequence provided at NCBI Ref. No. Plasmodb ID PF3D7_0109800 or Uniprot ID Q8I246_PLAF7 or NCBI Ref. No XP_001351028.1 having the exemplary sequence:

| | | | | | SEQ ID NO: 2 | |
|---|---|---|---|---|---|---|
| 1 | MSTNNVEEKK | NEELYNLLNI | LEEEFNLSKI | ENKNNNKNDD | GDDDDVNKRE | DNNNNEYYKK |
| 61 | LKDEKGLDVL | INEYTTSLKI | SKKYNIDHNK | VMGMIKKLET | LYYIINLIKS | FNTYHLSDEG |
| 121 | KQYLKEGSPE | FIVIKYVIQN | NKCTLEDLKK | NLGKIGDIGL | NTNLKNRTIQ | LNKADKCLTC |
| 181 | NCNLHDIQDF | TQIYLDIINQ | YGHDEELLFA | HLKNKNHTKN | ALNNNEMKNS | CKDNNIINNL |
| 241 | IQDLKKRKLL | EIKKNSYSHV | IKTSIFKKDI | KKQITDINYL | LLKNEEYKKY | DIKKYNFFSS |
| 301 | GKKINKGNIH | LLTKQMRTFK | EIFFSLGFEE | METHNYVESS | FWCFDALYIP | QQHPSRDLQD |
| 361 | TFFIKEPETC | IDKFVDTEYI | DNIKRVHTHG | DYGSFGWNYK | WKLEESKKNV | LRTHTTANSC |
| 421 | RALFKLAKEY | KEAGCIKPKK | YFSIDRVFRN | ENLDSTHLAE | FHQVEGLIID | KNIGLSHLIG |
| 481 | TLAAFYKHIG | IHKLKFKPTF | NPYTEPSMEI | YGYHEQSKKW | LEVGNSGIFR | PEMLRSMGFS |
| 541 | EEVSVIAWGL | SLERPTMIKY | NIKNIRDLFG | YKSVV. | | |

The apicomplexan FRS polypeptide may be a protein or fragment thereof having at least 85% amino acid sequence identity to the Plasmodium inui San Antonio 1 FRS α subunit sequence or fragment thereof, such as that provided at NCBI Ref No. XP_008818671 having the exemplary sequence:

| | | | | | SEQ ID NO: 3 | |
|---|---|---|---|---|---|---|
| 1 | MQAKAQEEQK | GEELSQFLQV | LEEEFALCQD | GGQKREEEEE | DDPWSASTEA | EELAKQRAYY |
| 61 | LQLKEEKNVQ | VEEAKHVTSL | HLSRKYHLEH | SKVLGMAKKL | ETLYYVVNHV | RSFNTYQLTD |
| 121 | EGKEYLRHGS | PEYVTLKYVV | EQQTCTLEDL | KKLFGKKGEI | GLNINLREKK | IQLGKSDRRL |
| 181 | YPHVEGKADS | LVDETRRYLG | IVETHGHDEG | TLVSHLKGIL | PPENQDEKVN | ILIRELKKRK |
| 241 | LMEVKKVSYM | YIIRTSHFRR | EIKKQITDLT | YQLIKNEEYK | KYEVKKYNFF | SSGKKINKGN |
| 301 | IHLLIKQMRT | FKDVFVSLGF | EEMDTHNYVE | SSFWCFDALY | IPQQHPSRDL | QDTFFVQTPE |
| 361 | LCRDNFTDKT | YIDNVKRVHS | VGDYGSFGWN | YPWELGSTKK | NVLRTHTTAN | SCRTLFQLAK |
| 421 | EYQKTGSIIP | KKYFSIDRVF | RNENLDSTHL | AEFHQVEGLI | VDKNLGLSHL | IGTLAAFYKY |
| 481 | IGIFKLRFKP | TFNPYTEPSM | EVYGYHEENK | KWMEVGNSGI | FRPEMLRAMG | FPPEVSVIAW |
| 541 | GLSLERPTMI | KYNIRNIRDL | FGYRSVF. | | | |

The apicomplexan FRS polypeptide may be a protein or fragment thereof having at least 85% amino acid sequence identity to the Plasmodium ovale FRS α subunit sequence provided or fragment thereof, such as that provided at NCBI Ref No. SBS80025.1 or Plasmodb ID PocGH01_02012500 or Uniprot ID A0A1D3KWQ1 (A0A1D3KWQ1_9APIC). An exemplary sequence is provided below:

| | | | | | SEQ ID NO: 4 | |
|---|---|---|---|---|---|---|
| 1 | MNCNNGMHGN | GGAEEELYSF | LSLLDDEFKL | CGTNDKGGSE | EKREYIAYYA | NLKEDKGIDV |
| 61 | MVNECTTSLH | LSKKYNIEHS | KVIGMIKKLE | TLYYVINHVK | SENTYHLTEE | GMKYIRDGSP |
| 121 | EYVVLKLVLE | KKKSTMEEVK | NLLGKKGEIG | LNTNLKNKHI | QLSKSDKSLS | SHLSLHNLED |
| 181 | KTKKCLEIVN | TYGQEEENLW | KVMKNIHNGD | NNEVNILIQD | LKKRKLLEVK | KNSYSYVVKT |
| 241 | SKFKKDIKKQ | ITDLNYLLLR | NDEYAKYDIK | KYNFFSSGKK | MNKGNIHLLT | RQMRAFKDIF |
| 301 | ISLGFEEMDT | QNYVESSFWC | FDALYIPQQH | PSRDLQDTFF | IKEPEMCADN | FIDSDYIANI |
| 361 | EKVHTYGDYG | SFGWNYKWKL | EESKKNVLRT | HTTANSCRTL | FKLAKEYNKK | GCIIPKKYFS |
| 421 | IDRVFRNENL | DSTHLAEFHQ | IEGLIIDKNL | GLSQLISTLS | AFYKYIGIHK | LKFKPTFNPY |
| 481 | TEPSMEIYGY | HEENKKWLEV | GNSGVFRPEM | LRSMGFEKDV | SVIAWGLSLE | RPTMIKYNIK |
| 541 | NIRDLFGYKS | VV. | | | | |

The apicomplexan FRS polypeptide may be a protein or fragment thereof having at least 85% amino acid sequence identity to the Plasmodium malariae FRS α subunit sequence provided or fragment thereof, such as that provided at NCBI Ref No. XP_028860155. An exemplary sequence is provided below:

| | | | | | SEQ ID NO: 5 | |
|---|---|---|---|---|---|---|
| 1 | MNANIVEDEQ | KNEELSRFIY | ILDKEFKLCK | EEAKKEEEAK | KEEEAKKDRG | GEKKEEQDEY |
| 61 | YSNLKKEKGI | EVLPNEYASS | LSISKKYDLD | HNKVVGLMKK | LEVLYYIINT | VKTFNTYHLT |
| 121 | EEGLQYLKEG | TPEYLVLKYV | VLEKKTCSIE | DLKKKFGKKG | DIGLNVNLKN | RTIQLNKTDK |
| 181 | SLSSSVDVNK | LQDVTQMYLS | LIDMHGHDEQ | LLMKEVERVG | LINDVITDGH | VSCVMQNLRK |
| 241 | RKLVEVKKNS | YCYVVKTSAF | KKDIKKQITD | LNYLLLKNEE | YKKYDIKEYN | FFSSGKKIIK |
| 301 | GTLHVLTKQM | RIFKDIFISL | GFEEMETHNY | VESSEWCFDA | LYIPQQHPSR | DLQDTFFIKD |
| 361 | PEICQDNFID | IGYIENVKRV | HSVGDYGSFG | WNYEWKIEES | KRNVLRTHTT | SNSCRTLFKL |
| 421 | AKEYNRKGHI | ILPKKYFSID | RVFRNENLDS | THLAEFHQVE | GLIIDKNLGL | AHLIGTLSAF |
| 481 | YKYIGIHKLK | FKPAFNPYTE | PSMEIFGYHE | QSRKWLEVGN | SGVFRPEMLR | AMGFPKDVSV |
| 541 | IAWGLSLERP | TMIKYNVKNI | RDLFGYRSTL. | | | |

The apicomplexan FRS polypeptide may be a protein or fragment thereof having at least 85% amino acid sequence identity to the Plasmodium vivax FRS sequence or fragment thereof, such as that provided at NCBI Ref No. XP_001616062.1 or Plasmodb ID PmUG01_02014200 or Uniprot ID A0A1A8XAF0 (A0A1A8XAF0_PLAMA). An exemplary sequence is provided below:

| SEQ ID NO: 6 | |
|---|---|
| 1 | MVLPLPKLIQ SNEGRVLYHI ANHPIRTVKK KIESFFKYES LDNLNSDISV RQNFDELFVP |
| 61 | LTHPARNIKD TFYLCKNYVK YFSTLHNDLY TRLENTNGIY KYYLTTKLVG HHKILLKRTH |
| 121 | MTAHLPDLLR RNYKNVIYTG SVYRRDEIDR FHFPIFHQTD GFLIRPDNFD VETDLKTNLQ |
| 181 | QLIRYLFDAS DIKMKWDGNT SFPFTDPSYE LYIRGVPSGG RGTTRDDNNG GAASASLQGD |
| 241 | PRGGEPPWVE VLGCGKIKKE VIAMALHAED IRQMIEDEIA RHDRGLLGEL HSMEAAASGA |
| 301 | AAEGTAPNEV TPNVATPNPA TSNVAPPKGI LDKLCSAPLN NRIETQMNKF LKTIRYQGWA |
| 361 | FGIGLERLAM LLYHIHDIRL FWSTDSRFID QFEEDVITKF HPFSNFPPVE KDISFYVSSQ |
| 421 | FKEALFFQIC RDIASENIEQ VKKIDSYHNP RNNQTSVCYR ITYRSHSQNL THKAVNELQS |
| 481 | KIAQMLVKQC AVTIR. |

The apicomplexan FRS polypeptide may be a protein or fragment thereof having at least 85% amino acid sequence identity to the Plasmodium vivax FRS α subunit sequence or fragment thereof such as that provided at NCBI Ref No. XP_001613535.1 or Plasmodb ID PVX 081300 or Uniprot ID A5K9S0 (A5K9S0_PLAVS). An exemplary sequence is provided below:

| SEQ ID NO: 7 | |
|---|---|
| 1 | MQAKAQEEQK GEELSQFLQA LEEEFALCQE GGGDKREADG PSLENAEAEE LANRRAYYLQ |
| 61 | LKEERNVLVE ESKHVTSLHM SSKHNIEHAK VLGMAKKLET LYYVVNHVRS FNTYQLTEEG |
| 121 | KEYLRDGSPE HVTLRYVMEQ EGCTLEDLKK LFGKKGEIGL NINLKKKKIE LRKSDKRLFP |
| 181 | HVEGSAHSLV DETRCYLQKV EAHGNDEGAL VSHLKGILPP EKGEKKEEDE ANNLIKELKK |
| 241 | RKLIEAKKIS YIYIIRTNLF TKEIKKQITD LTYLLIKNEE YKKYQVKKYN FFSSGKKMNK |
| 301 | GNIHLLIRQM RTFKDVFVSL GFEEMNTHNY VESSFWCFDA LYIPQQHPSR DLQDTFFIKV |
| 361 | PEMCQEEFTD QSYIENVKRV HSVGDYGSFG WNYQWELKST KKNVLRTHTT ANSCRALFQL |
| 421 | AKEYQKTGSI IPKKFFSIDR VFRNENLDST HLAEFHQVEG LIIDRNLGLS HLIGTLSAFY |
| 481 | KYIGISKLRF KPTFNPYTEP SMEVYGYHEE NKKWLEVGNS GIFRPEMLRA MGFPPEVSVI |
| 541 | AWGLSLERPT MIKYSIRNIR DLFGYRSVI. |

The apicomplexan FRS polypeptide may be a protein or fragment thereof having at least 85% amino acid sequence identity to the Cryptosporidium hominis FRS α subunit sequence or fragment thereof such as that provided at NCBI Ref No. XP_668177.1 or Cryptyo db ID Chro.30378. An exemplary sequence is provided below:

| SEQ ID NO: 8 | |
|---|---|
| 1 | MEVEPVTILE KLESLNCSLS SISISEELKC EHERVIGVIK SLESRNIIKT QFISDNMLSL |
| 61 | TDEGKDYLLN GSPEYRLIKI ILEKSDHKIK QDEAMTFLGG SIFKIGLSKC LQKKLIKLNK |
| 121 | ELGLIEIPAD SNMDLLNTDE LGKNLFIVGV NGIKESQLTK QDLQIITELK KRKLVVSQKM |
| 181 | SYFMINKGED FTVKLKPMIT DLTQEMIMNS SWENNEFKPY NFNAKGKRLP RGNIHPLTRT |
| 241 | SRKFKRILSQ MGFEEMPTNR WVESSFWNFD ALFQPQKHPA RDSHDTFFLE TPSTFRNEME |
| 301 | LSNDHIDKVK KVHEVGGYGS IGLSYNWSIE EASKNLLRTH TTAVSVRMLY KLAQMYLYNE |
| 361 | NGLSFDNFQR KAYFSIDRVE RNESIDATHL AEFHOVEGLI VDKNLTLADL IGTLKTFYEK |
| 421 | IGISDLKFKP AFNPYTEPSM EIFGYHTTLK KWIEVGNSGL FRPEMLRPLG FPSDIVVIAW |
| 481 | GLSLERPTMI SYNIPNIRDL FSFKAKIFS. |

The apicomplexan FRS polypeptide may be a protein or fragment thereof having at least 85% amino acid sequence identity to the Toxoplasma gondii FRS α subunit sequence or fragment thereof such as that provided at NCBI Ref No. XP_002368890.1 or Toxo DB ID TGME49_234505 or Uniprot ID (A0A125YPN6_TOXGM). An exemplary sequence is provided below:

| SEQ ID NO: 9 | |
|---|---|
| 1 | MADVASAALM DDFLQVLNKE FGLQPVVSTL ALAENSEYGG KFGAHEKIVG LVKSLQANEY |
| 61 | VLGEQVEKTV WSLTEEGQLY AKEGSPEFRL VAWLQQRGNE EASLDVAAIK AGFGSEADVA |
| 121 | VSNAMKMQWL RIDKATKLLH INKVPESDNV KTLLDVIQGQ RDGNQDDAAA LFKQLSELTA |
| 181 | TASPEKALQD LKKRKLVELR KVKYLKLQKG PKFSLHLMKP VADLTAELLI GDAWEKSNFK |
| 241 | EYNFFAAGKR IRRGAVHPLM QVMKQFKQIL YCMGFEEMPT NQYVESSFWC FDSLFMPQQH |
| 301 | PARDVQDTFF LQAPKSSDAT KIPQAYFNSV KNIHERGGHG SIGWQYEWSE EESMGNILRT |
| 361 | HTTASSARML YGLAQEYKKT GVFRPRKFFS IDRVERNETL DATHLAEFHQ VEGLVADRGL |
| 421 | TIGHLMGVME TFYKQIGIEQ LKFKPAYNPY TEPSMEIFGY HAGLKRWIEV GNSGIFRPEM |
| 481 | LLPMGLPEDV TVIAWGLSLE RPTMIRYGIS NIRQLFGHRA LLGMA. |

The apicomplexan FRS polypeptide may be a protein or fragment thereof having at least 85% amino acid sequence identity to the Plasmodium falciparum: FRS β subunit sequence or fragment thereof, such as the sequence provided at NCBI Ref. No. XP_001347727.1 or Plasmodb ID PF3D7_1104000 or Uniprot ID Q8IIW2_PLAF7 having the exemplary sequence:

| SEQ ID NO: 10 | |
|---|---|
| 1 | MPTISVYEDD LFEKLGEEII EEKLLDVCFD FGLEVDDIEY KNDKKIYKIE VPANRYDLIC |
| 61 | VEGLCRALKN FMCKFDDIKY DISMNNYDIC IKGNQYIKVD GSVDDRRGYV VCCVLKNMNI |
| 121 | NDSVYNNIIE IQEKLHHNLG KKRSVLAIGI HDYDKIKFPL KYKFEKKEKI NFIPLNEKTN |
| 181 | LNGMNLIDFY SKNLNLKPYL KIIKDFDKYP IIVDSNEQIL SLPPIINCDH TKISLNTKNV |
| 241 | FIECTAIDRN KAQIALNILC SMLSEYCVPK YSIQSFVVIY ENQDFSDDQN LKKKETQFLY |
| 301 | PIFENKSLTC NIDYVRKLSG ISHITVHEVN NLLKRMMLSC DIMDNNTFKV TIPFYRSDIM |
| 361 | HCCDIIEDIA IAYGYGNIKY EPPQICKKHS LNNCSELFRN VLVECGYTEV MTNALLSRDE |
| 421 | NYNCMLRTHK SYDDPNINLD EYNPLAAPIQ IKNSKTSEYE IIRTSLIVNL LKFVSANKHR |
| 481 | ELPLRFFEIG DVSYATYNQT DTNAVNKKYL SIIFSDKFTA GLEELHGVLE AILKEYQLFS |
| 541 | DYKIEEKKKE NISIRSDMYY KLIPKEDPSF LNERIVDIVL FPHNLKFGVL GIIHPKVLEN |
| 601 | FSLDIPVSAI EINVETLLNV LMM. |

The apicomplexan FRS polypeptide may be a protein or fragment thereof having at least 85% amino acid sequence identity to the PvFRS β subunit sequence or fragment thereof such as that provided at NCBI Ref No. XP_001615169.1 or Plasmodb ID PVX_090880 or Uniprot ID A0A1G4GX19 (A0A1G4GX19_PLAVI). An exemplary sequence is provided below:

| SEQ ID NO: 11 | |
|---|---|
| 1 | MPTISVHEED LIEKLGEKIE EEKLNDICFE FGIEIDDVEY KGEKKIYKIE VPANRYDLVC |
| 61 | VEGLCRALKS FIGKYENVSY ALLTNSEEAC VKEKHFMRVD ESVDERRSYV VSAVLKNVKM |
| 121 | NENVYNNIIE LQEKLHHNLG KKRILLAIGI HDYDKINFPV AYKFEEKEKI NFIPLNETQN |
| 181 | VNGNNFINFY QDNINLKSYL KIISDFEKFP VIVDAGGQIL SLPPIINCDY TKITYDTRNL |
| 241 | FIECTAIDRN KAEIAVNIIC SMLSEYCTPK YSIHSFFVQY DKNHKAEKGN GYLYPVFKNK |
| 301 | TLTCHMDYVR KLSGILNLSV KDVEPLLKKM MITSKVIDSS TFTVDVPFYR SDIMHFCDIV |
| 361 | EDIAIAYGYG NIVSEKIEIA KKNSLSACTE LFRNVLAECT YTEVMTNALL SKRENYDCML |
| 421 | RKHRSYDDRK INLDEYNPLA PPVQIMNSKT SEYEIVRTSL IVNMLKFVSA NKHRELPLRF |
| 481 | FEIGDVSYTT YDRTDTNAVN KRYLSVIFAD KFTAGLEEAF GMLETVLKEF QLFSDYKIEE |
| 541 | KSKENVAIRS DVFYKLVPKE DPSFLNERVV DIVLCPHNLK FGIMGIIHPK VLENFSIDIP |
| 601 | VSVIEINIET IMDVLMM. |

The apicomplexan FRS polypeptide may be a protein or fragment thereof having at least 85% amino acid sequence identity to the Plasmodium fragile FRS β subunit sequence or fragment thereof such as that provided at NCBI Ref. No. XP_012336572. An exemplary sequence is provided below:

| SEQ ID NO: 12 | |
|---|---|
| 1 | MPTISVYEED LIEKLGQKIE EEKLNDICFE FGIEIDDIEY KGEKKIYKIE VPANRYDLVC |
| 61 | VEGLCRALKS FIGKCQNINY VLLQNSEEAC VKEKHFIRVD ESVDERRSYV VSAVLKNVKI |
| 121 | NENVYNNIIE LQEKLHHNLG KKRILLAIGI HDYDKIKFPV VYKFEEKDKI NFIPLNETKN |
| 181 | VNGNNFLKFY QDNINLKSYL KIIKDFEKFP VIVDADNKIL SLPPIINCDH TKITYDTKNL |
| 241 | FIECTAIVKN KAEIAVNIIC SMLSEYCTPK YSIHSFFVQY DKKHKAEKGN GYLYPIFKNK |
| 301 | SLTCHMDYVR KLSGILDLSV KDVEPLLKKM MISSKIIDSN TFTVDVPFYR SDIMHCCDIV |
| 361 | EDIAIAYGYG NIVSEKIEIA KKNSLSACTE LFRNVLVECT YTEVMTNALL SKRENYDCML |
| 421 | RKHRDYKDEK INLDEYNPLA PPVQIMNSKT SEYEIVRTSL IVNMLKFVSA NKHRELPLRF |
| 481 | FEIGDVSYTT YNKTDTNAVN KRYLSVIFAD KFTAGLEEAH GMLETVLKEF QLFSDYKIEE |
| 541 | KRKENVAIRS DVFYKLVPTK DPSFLNERVV DIILCPQNLK FGILGIIHPK VLENFSIDVP |
| 601 | VSVMEINIES IMNILMI. |

The apicomplexan FRS polypeptide may be a protein or fragment thereof having at least 85% amino acid sequence identity to the Plasmodium gonderi FRS β subunit sequence or fragment thereof such as that provided at NCBI Ref. No. XP_028861466.1 An exemplary sequence is provided below:

| SEQ ID NO: 13 | |
|---|---|
| 1 | MPTISVFEED LIEKLEERLS DEKLQDICFD FGLEVDDIEY KNKKKIYKIE VPANRYDLVC |
| 61 | VEGLCRALKS FIGKFDNIKY DILMNSHECC IKEKHYINVK ESVDERRSYV VSCVLKNIKM |
| 121 | TEHVYNNIIE LQEKLHHNIG KKRTVLAIGI HDYDKIKFPV EYKFEEKSKI NFIPLNETKN |
| 181 | LNGNNLMEFY EHNTNLKPYL KIIKNFEKYP LIVDANNQIL SLPPIINCEY TKITLNTKNL |
| 241 | FVECTATDKH KAEIALNIIC SMLSEYCTPK YSIYSFLVLY DQNHKIEKGN SYLYPEFKNK |
| 301 | VVTCEIDYVR KLSGIPNITV EDVEKVLKKM MIPIKIIDNC TFTAHVPFYR SDIMHSCDIV |
| 361 | EDIAIGYGYG NIKSCEVEFS KKHLLNTCSD LFRNALIECA YTEVMTNALL SKRENYNCML |
| 421 | RKYKDYKDVQ INLEEYNPLA PPVQIMNSKT SEYEIIRTSL IVNLLKFVAS NKHRELPLRF |
| 481 | FEIGDVSYTT YNKTDTNAVN KRHLSVIFAD KFCSGLEEIH GVLETVLKEF QLENDYKIDE |
| 541 | KKKENISLRS DVFYKLLPKE DSSFLSERVV DIVLFPHNLK FGVLGIIHPE VLGNFSIDIP |
| 601 | VSAVEINIET LLNVLMM. |

The apicomplexan FRS polypeptide may be a protein or fragment thereof having at least 85% amino acid sequence identity to the Plasmodium gonderi FRS β subunit sequence or fragment thereof such as that provided at NCBI Ref. No. XP_028541670.1 An exemplary sequence is provided below:

| SEQ ID NO: 14 | |
|---|---|
| 1 | MNKKEKGNQN VEKNENTFEE QKEDEMNHFL KVLEEEFGLC REKYEKKEDV QMNEFGIDVA |
| 61 | SHRSYYLQLK DEKNIEVLEN EHTTSLCVSE KHNLEHSKVL GMVKKLETLY YVINHVKSFN |
| 121 | KYQLTEEGEE YLKSGSPEYV TLKFVVENEK CTLDHLKKMF GKKGEIGLNI NLKNKKIELN |
| 181 | KNDKSLIPRV STTGCDIVDD TRKLLNVIKA EGHDEHMLFT HLKETLFSKL DSDVNNLIAN |
| 241 | LKKRKLMEVK KVSYSYIIRT SRFEKKIKKQ ITDMNYQLMK NEEYKKYDIK KYNFFSSGKK |
| 301 | INKGNIHLLT KQMRTFKEIF ISLGFEEMET HNYVESSFWC FDALYIPQQH PSRDLQDTFF |
| 361 | IKIPELCNEN FIDSEYIENV KRVHSVGDYG SFGWNYNWEL RESKKNVLRT HTTANSCRAL |
| 421 | FKLAKEYKKT GSIIPKKYFS IDRVFRNENL DSTHLAEFHQ VEGLIIDKNL GLSHLIATLA |
| 481 | SFYKYIGIHK LRFKPTFNPY TEPSMEVYGY HEQNKKWIEV GNSGIFRPEM LQAMGFSDEV |
| 541 | SVIAWGLSLE RPTMIKYNIR NIRDLFGYRS LIA. |

The apicomplexan FRS polypeptide may be a protein or fragment thereof having at least 85% amino acid sequence identity to the Plasmodium falciparum: FRS β subunit sequence or fragment thereof such as that provided at Plasmod_id PF3D7_1104000 or Uniprot_id Q8IIW2_PLAF7 or NCBI Ref. No XP_001347727.1 having the exemplary sequence:

| SEQ ID NO: 15 | |
|---|---|
| 1 | MPTISVYEDD LFEKLGEEII EEKLLDVCFD FGLEVDDIEY KNDKKIYKIE VPANRYDLIC |
| 61 | VEGLCRALKN FMCKFDDIKY DISMNNYDIC IKGNQYIKVD GSVDDRRGYV VCCVLKNMNI |
| 121 | NDSVYNNIIE IQEKLHHNLG KKRSVLAIGI HDYDKIKFPL KYKFEKKEKI NFIPLNEKTN |
| 181 | LNGMNLIDFY SKNLNLKPYL KIIKDFDKYP IIVDSNEQIL SLPPIINCDH TKISLNTKNV |
| 241 | FIECTAIDRN KAQIALNILC SMLSEYCVPK YSIQSFVVIY ENQDFSDDQN LKKKETQFLY |
| 301 | PIFENKSLTC NIDYVRKLSG ISHITVHEVN NLLKRMMLSC DIMDNNTFKV TIPFYRSDIM |
| 361 | HCCDIIEDIA IAYGYGNIKY EPPQICKKHS LNNCSELFRN VLVECGYTEV MTNALLSRDE |
| 421 | NYNCMLRTHK SYDDPNINLD EYNPLAAPIQ IKNSKTSEYE IIRTSLIVNL LKFVSANKHR |
| 481 | ELPLRFFEIG DVSYATYNQT DTNAVNKKYL SIIFSDKFTA GLEELHGVLE AILKEYQLFS |
| 541 | DYKIEEKKKE NISIRSDMYY KLIPKEDPSF LNERIVDIVL FPHNLKFGVL GIIHPKVLEN |
| 601 | FSLDIPVSAI EINVETLLNV LMM. |

The apicomplexan FRS polypeptide may be a protein or fragment thereof having at least 85% amino acid sequence identity to the Cryptosporidium hominis FRS β subunit sequence or fragment thereof such as the sequence provided at NCBI Ref. No. OLQ17121.1 An exemplary sequence is provided below:

| SEQ ID NO: 16 | |
|---|---|
| 1 | MEVEPVTILE KLESLNCSLS SISISEELKC EHERVIGVIK SLESRNIIKT QFISDNMLSL |
| 61 | TDEGKDYLLN GSPEYRLIKI ILEKSDHKIK QDEAMTFLGG SIFKIGLSKC LQKKLIKLNK |
| 121 | ELGLIEIPAD SNMDLLNTDE LGKNLFIVGV NGIKESQLTK QDLQIITELK KRKLVVSQKM |
| 181 | SYFMINKGED FTVKLKPMIT DLTQEMIMNS SWENNEFKPY NFNAKGKRLP RGNIHPLTRT |
| 241 | SRKFKRILSQ MGFEEMPTNR WVESSFWNFD ALFQPQKHPA RDSHDTFFLE TPSTFRNEME |
| 301 | LSNDHIDKVK KVHEVGGYGS IGLSYNWSIE EASKNLLRTH TTAVSVRMLY KLAQMYLYNE |
| 361 | NGLSFDNFQR KAYFSIDRVF RNESIDATHL AEFHQVEGLI VDKNLTLADL IGTLKTFYEK |
| 421 | IGISDLKFKP AFNPYTEPSM EIFGYHTTLK KWIEVGNSGL FRPEMLRPLG FPSDIVVIAW |
| 481 | GLSLERPTMI SYNIPNIRDL FSFKAKIFS. |

The apicomplexan FRS polypeptide may be a protein or fragment thereof having at least 85% amino acid sequence identity to the Cryptosporidium hominis FRS β subunit sequence or fragment thereof such as the sequence provided at NCBI Ref. No. OLQ17346.1 An exemplary sequence is provided below:

| SEQ ID NO: 17 | |
|---|---|
| 1 | MPVVSVSTRV LSSYLNKEVT SEWLDEVCFN FGLELDCIEF DEEIKDNVAK IEIPANRPDI |
| 61 | LCLEGLVIAL GCFIGNSNIP LFNLKPKGDD QKIIVKRNVA RVRPFILCAI LRDLSFNEDI |
| 121 | YRSFIDYQEK LHNNICRKRS LVSIGTHDLD MIEGPFYYDA KAHSEIKFVP LIGSAEVDGN |
| 181 | QLLDLLNKHQ QLKKYVSLVE NEIFLPVVTD SNGIVLSVPP LINGSQSKIT LNTKNVFIEV |
| 241 | TATDYNRASI VLNQIVSSFS MYCKNKFEIE PVKVEYEHST YPPFPHKYEV LANGNYHVVT |
| 301 | PNVERLEFVV KASEASNLLG INPILDPIVA QSLLRKMMID SEIIKDNESL KCFVPINRSD |
| 361 | ILHPVDIIED IGISFGFNNI ELKKSRFYEL DKSNLMAEQV KRELSLLGIS ESLNWALCKH |
| 421 | SDCFESLFRT ENIKLKNLFE SEVQYNLNCP PVVVKDAKTS EFEILRTTLI QSLLKTIASN |
| 481 | KSLPLPQKIF EVGDVVILDN NTPSGSRNDK RVAIAYCNSS GSGLEEIHGF LDQLLSKLGL |
| 541 | VAEYSLNEPN SIHPNIIGMY SLSEVNDPSF LPTRSVKITI QKVELNSCLK SIDIEKTMLN |
| 601 | KQVVIGIMGV VHPKVLNNFS LTLPTSVLEL RLEPIMHLWP DTFFYDE. |

The apicomplexan FRS polypeptide may be a protein or fragment thereof having at least 85% amino acid sequence identity to the Toxoplasma gondii FRS β subunit sequence or fragment thereof such as the sequence provided at NCBI Ref. No. KYF47306.1 having the exemplary sequence:

| SEQ ID NO: 18 | |
|---|---|
| 1 | MPTVSVPRDE LFRRLGRTYS VHEFEELCFE FGIELDEVVE PGKDGSTETI YKIEVPANRY |
| 61 | DLLCTEGISR ALYAFNNPDA PLPAYRLEPA TPQFTMTVKP AVNQVRPFVV CAILRNVTLT |
| 121 | KAGLASFIEF QDKLHHTLCR RRSLVAIGTH DLSKIQPPFV YDARPPKNFE FVPLGCDSQM |
| 181 | NGEQVMAHFS SHLQLKAYLP LIQNSPVYPL ILDAKDRILS LPPIINSEFS KVTEDTRDIF |
| 241 | IECTAVDITK AQIVLNTLVA MFSEYCKEPY TVEPIRVVYE DPSSAPIDRS VKCQGEASLQ |
| 301 | NGSASMNGWV FPRVNSRSMP FSLDYVRQLT GIPDLTADAC ANLLKRMMIH TSIEKATQAG |
| 361 | ILEASIPITR SDILHERDIV EDVAIAYSFN RLPVTRSYML TGDALNCLSE KIRNFCTVCG |
| 421 | YTEALNFSLS SAAENSSSLG RTPGDGKSSL FNPLEYQVNA QPVRLANSKT REFDQVRTTL |
| 481 | ISGLLKTIVA NKGRRELPIK LFEIGDVCLL DSTTEVGARN LRYCCLAFAD EHSSGLEEVH |
| 541 | GVLDALLQSL QFVGEYAIAE MEAAVAAAAA AEKAGCEHAS AHATEQLQRT LSRIRGTFKL |
| 601 | VPSHEETFLP GRQVQVVASL KSSGDMENVP VLLGVMGTLH PHTLKAFGLT IPVSIFELNV |
| 661 | EAFVQWLPAI DLVVG. |

As used herein “Human phenylalanyl-tRNA synthetase polypeptide” or “Human FRS polypeptide” refers to polypeptides of the phenylalanyl-tRNA synthetase enzyme of a Homo sapiens (Hs). The polypeptide may include dimers of the alpha and beta subunits including heterodimeric forms which may optionally further dimerize to include heterotetramers (e.g., α2β2).

The human FRS polypeptide may be a protein or fragment thereof having at least 85% amino sequence identity to the Homo sapiens FRS α subunit sequence or fragment thereof such as the sequence provided at NCBI Ref. No. NP_004452.1 or Uniprot ID Q9Y285. An exemplary sequence is:

| SEQ ID NO: 19 | |
|---|---|
| 1 | MADGQVAELL LRRLEASDGG LDSAELAAEL GMEHQAVVGA VKSLQALGEV IEAELRSTKH |
| 61 | WELTAEGEEI AREGSHEARV FRSIPPEGLA QSELMRLPSG KVGFSKAMSN KWIRVDKSAA |
| 121 | DGPRVFRVVD SMEDEVQRRL QLVRGGQAEK LGEKERSELR KRKLLAEVTL KTYWVSKGSA |
| 181 | FSTSISKQET ELSPEMISSG SWRDRPFKPY NFLAHGVLPD SGHLHPLLKV RSQFRQIFLE |
| 241 | MGFTEMPTDN FIESSFWNFD ALFQPQQHPA RDQHDTFFLR DPAEALQLPM DYVQRVKRTH |
| 301 | SQGGYGSQGY KYNWKLDEAR KNLLRTHTTS ASARALYRLA QKKPFTPVKY FSIDRVFRNE |
| 361 | TLDATHLAEF HQIEGVVADH GLTLGHLMGV LREFFTKLGI TQLRFKPAYN PYTEPSMEVE |
| 421 | SYHQGLKKWV EVGNSGVFRP EMLLPMGLPE NVSVIAWGLS LERPTMIKYG INNIRELVGH |
| 481 | KVNLQMVYDS PLCRLDAEPR PPPTQEAA. |

The human FRS polypeptide may be a protein or fragment thereof having at least 85% amino sequence identity to the Homo sapiens FRS β subunit sequence or fragment thereof such as the sequence provided at NCBI Ref. No. NP_005678.3 or Uniprot ID Q9NSD9. An exemplary sequence is:

| SEQ ID NO: 20 | |
|---|---|
| 1 | MPTVSVKRDL LFQALGRTYT DEEFDELCFE FGLELDEITS EKEIISKEQG NVKAAGASDV |
| 61 | VLYKIDVPAN RYDLLCLEGL VRGLQVFKER IKAPVYKRVM PDGKIQKLII TEETAKIRPF |
| 121 | AVAAVLRNIK FTKDRYDSFI ELQEKLHQNI CRKRALVAIG THDLDTLSGP FTYTAKRPSD |
| 181 | IKFKPLNKTK EYTACELMNI YKTDNHLKHY LHIIENKPLY PVIYDSNGVV LSMPPIINGD |
| 241 | HSRITVNTRN IFIECTGTDF TKAKIVLDII VTMFSEYCEN QFTVEAAEVV FPNGKSHTFP |
| 301 | ELAYRKEMVR ADLINKKVGI RETPENLAKL LTRMYLKSEV IGDGNQIEIE IPPTRADIIH |
| 361 | ACDIVEDAAI AYGYNNIQMT LPKTYTIANQ FPLNKLTELL RHDMAAAGFT EALTFALCSQ |
| 421 | EDIADKLGVD ISATKAVHIS NPKTAEFQVA RTTLLPGLLK TIAANRKMPL PLKLFEISDI |
| 481 | VIKDSNTDVG AKNYRHLCAV YYNKNPGFEI IHGLLDRIMQ LLDVPPGEDK GGYVIKASEG |
| 541 | PAFFPGRCAE IFARGQSVGK LGVLHPDVIT KFELTMPCSS LEINVGPFL. |

By “computer readable media” is meant any media which can be read and accessed directly by a computer, for example, so that the media is suitable for use in the computer systems described herein. The media may include magnetic storage media such as floppy discs, hard disc storage medium and magnetic tape; optical storage media such as optical discs or CD-ROM; electrical storage media such as RAM and ROM; and hybrids of these categories such as magnetic/optical storage media.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

By “disease” is meant any condition or disorder that damages or interferes with the normal function of a cell, tissue, or organ. Examples of diseases include diseases associated with apicomplexan infection, such as malaria, toxoplasmosis, and cryptosporidiosis.

“Detect” refers to identifying the presence, absence or amount of the analyte to be detected.

The term “effective amount” or “therapeutically effective amount” of an agent is meant the amount of an agent (e.g., a compound described herein) required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. Agents described herein include compounds having the structure of Formula (I), Formula (II), Formula (III), Formula (IV), or those identified in the screening assays of the present disclosure. In some embodiments, the compounds are administered in an effective amount for the treatment or prophylaxis of a disease disorder or condition. In another embodiment, in the context of administering an agent that is an antiparasitic agent, an effective amount of an agent is, for example, an amount sufficient to achieve alleviation or amelioration or prevention or prophylaxis of one or more symptoms or conditions; diminishment of extent of disease, disorder, or condition associated with parasitic infection; stabilized (i.e., not worsening) state of disease, disorder, or condition; preventing spread of disease, disorder, or condition; delay or slowing the progress of the disease, disorder, or condition; amelioration or palliation of the disease, disorder, or condition (e.g., those associated with infection); and remission (whether partial or total), whether detectable or undetectable, as compared to the response obtained without administration of the agent. In some embodiments, the antiparasitic agent slows the rate of infection or decreases the number of parasites in a host subject or infected medium.

The term “pharmaceutical composition,” as used herein, represents a composition containing a compound described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap); for topical administration (e.g., as a cream, gel, lotion, or ointment); for intravenous administration (e.g., as a sterile solution free of particulate emboli and in a solvent system suitable for intravenous use); or in any other formulation described herein (see below).

As used herein, the phrase “pharmaceutically acceptable” generally safe for ingestion or contact with biologic tissues at the levels employed. Pharmaceutically acceptable is used interchangeably with physiologically compatible. It will be understood that the pharmaceutical compositions of the disclosure include nutraceutical compositions (e.g., dietary supplements) unless otherwise specified.

By “reduces” is meant a negative alteration of at least 10%, 25%, 50%, 75%, or 100%.

By “reference” is meant a standard or control condition.

A “reference sequence” is a defined sequence used as a basis for sequence comparison. In some embodiments, a reference sequence is the sequence of an apicomplexan FRS polypeptide provided herein. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “subject” is meant a mammal, including, but not limited to, a human or non-human mammal, such as a bovine, equine, canine, ovine, or feline. Typical subjects include any animal (e.g., mammals such as mice, rats, rabbits, non-human primates, and humans). A subject in need thereof is typically a subject for whom it is desirable to treat a disease, disorder, or condition as described herein. For example, a subject in need thereof may seek or be in need of treatment, require treatment, be receiving treatment, may be receiving treatment in the future, or a human or animal that is under care by a trained professional for a particular disease, disorder, or condition.

By “substantially identical” is meant a polypeptide or nucleic acid molecule exhibiting at least 50% identity to a reference amino acid sequence (for example, any one of the amino acid sequences described herein) or nucleic acid sequence (for example, any nucleic acid sequence encoding a polypeptide described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “substituent” refers to a group “substituted” on a hydrocarbon, e.g., an alkyl, aryl, cycloalkyl, alkylene, arylene, cycloalkylene, heteroalkyl, heteroaryl, heterocyclo, heteroalkylene, heteroarylene, heterocycloene, at any atom of that group, replacing one or more atoms therein (e.g., the point of substitution) including hydrogen atoms. In some aspects, the substituent(s) on a group are independently any one single, or any combination of two or more of the permissible atoms or groups of atoms delineated for that substituent. In another aspect, a substituent may itself be substituted with any one of the substituents described herein. Substituents may be located pendant to the hydrocarbon chain.

In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different (e.g., R may be independently selected at each occurrence from C₁-C₁₀ alkyl optionally comprising one or more points of substitution).

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 shows the inhibition of the aminoacylation activity of Pf(Plasmodium falciparum), Pv (Plasmodium vivax), Hs (Human) and Pf-Mut (L550V) cFRS enzymes by Compound 1. These assays were performed at concentrations ranging from 0.001 nM to 100 M and the IC50 values were calculated by non-linear regression. The error bars indicate standard deviation (n=3).

FIG. 2A is a Lineweaver-Burk plot obtained at a saturating concentration of ATP (500 μM) with varying concentrations of L-Phe (0.001-10 μM) and Compound 1 (1×IC₅₀ (triangle), 0.5×IC50 (square), and 0×IC50 (black circle). The error bars indicate standard deviation (n=3).

FIG. 2B is a Lineweaver-Burk plot obtained at a saturating concentration of L-Phe (2 μM) with varying concentrations of ATP (0.001-10 μM) and Compound 1 (1×IC50 (triangle), 0.5×IC50 (square), and 0×IC50 (vehicle control, black circle). The error bars indicate standard deviation (n=3).

FIG. 3A shows the final purified PfcFRS and PvcFRS proteins as measured by SDS-PAGE illustrating the separation of the α and β subunits in the electrophoretic gel.

FIG. 3B is a sensogram of the binding of Compound 1 to PfcFRS.

FIG. 3C is a sensogram of the binding of Compound 1 to HscFRS.

FIG. 3D is the GPC elution profile of Pf and Pv-cFRS. Comparison with standard marker shows that the Pf and Pv cFRS eluted at the size correspond to that of a tetramer.

FIG. 4A illustrates the structural domain organization of α- and β-subunits of PvcFRS.

FIG. 4B shows the overall structure of PvcFRS showing functional biological heterotetrametric assembly having two crystallographic heterodimer. The domain boundaries are labelled according to the TtFRS (Protein Data Bank Entry (PDB) 2IY5).

FIG. 5A shows the surface view of heterodimeric assembly of PvcFRS (linker and 0-subunits are boxed, α-subunit is not) structures. The subdomains of β1 and β2 of β-subunit are also indicated.

FIG. 5B shows the surface view of heterodimeric assembly of HscFRS (linker and 0-subunits are boxed, α-subunit is not) structures. The subdomains of β1 and β2 of β-subunit are also indicated.

FIG. 5C is an image with superimposed structures of Pv/HscFRS displaying movement of subdomain β2 of the α-subunit and hence the domain swap in the enzyme.

FIG. 5D is a close-up view of β-subunit linker region indicating the three residue insertion (boxed) in HscFRS, which, when absent lead to domain swap illustrated in FIG. 5C.

FIG. 5E is a surface view of the heterotetrametric assembly of the PvcFRS structure with the surface of each subunit depicted in different shades of grey (and boxed for clarity).

FIG. 5F is a surface view of the heterotetrametric assembly of HscFRS structures with the surface of each subunit depicted in different shades of grey.

FIG. 5G shows the portion of the sequence alignment showing the linker region between β1 and ρ2 subdomains of the β-subunit in malaria parasites Pv-β (SEQ ID NO: 21) and Pf-β (SEQ ID NO: 22 versus human cFRS (Hs-β) enzymes (SEQ ID NO: 23). The Hs linker is underlined.

FIG. 6A is a surface view of PvcFRS: Compound 1 complex having α-subunit and β-subunits with bound Compound 1 (dark grey) in the α-subunit. The view is magnified on the right illustrating that Compound 1 occupies the PvcFRS active site pocket. A simulated annealing omit map is shown on the magnified image with Fourier coefficients 2Fₒ-F_{c}, and contoured at 1 σ level.

FIG. 6B is a surface view of the active site and pockets within: L-Phe site, ATP site, and auxiliary site. Superimposed structures of L-Phe and phenylalanyl-adenylate are depicted.

FIG. 6C is a close-up view of amino acid and ATP pocket of PvcFRS (amino acid indicated before each slash) superimposed with HscFRS-L-Phe (PDB 3L₄G) (amino acid indicated after each slash) and T. thermophilus FRS-Phe-AMP-tRNA (PDB 2IY5). The overlayed HscFRS-L-Phe (PDB 3L4G) and T. thermophilus—FRS-Phe-Amp-tRNA has significant overlap with the bound Compound 1.

FIG. 6D is a close-up view of auxiliary site occupied by Compound 1. Auxiliary site residues are shown which are involved in protein-ligand interactions.

FIG. 6E is a rationalized chemical structure of Compound 1 highlighting its [6.2.0]-diazabicyclodecane core, and 4-cyclopropoxyphenyl, diarylacetylene, and methoxymethyl appendages.

FIG. 6F is a two-dimensional representation of Compound 1 binding in PvcFRS. Compound 1 is shown in ball-and-stick representation and residues engaging in hydrophobic interactions with the ligand are highlighted with dashed circles.

FIG. 7A shows a superimposition of HscFRS (PDB 3L₄G) and TtFRS (PDB 2IY5) structure with the PvcFRS: Compound 1 complex.

FIG. 7B illustrates the opening of Loop 1 (residues 442-453) of PvcFRS on Compound 1 binding to accommodate its methoxymethyl group. His451 in PvcFRS: Compound 1 adopts an open conformation.

FIG. 7C shows the closing of Loop 2 (residues 507-513) in PvcFRS: Compound 1 complex brings His508 in close proximity.

FIG. 7D shows the open conformation of the Arg548 in PvcFRS: Compound 1 complex accommodates its [6.2.0]-diazabicyclodecane core as compared to its orthologue residue in HscFRS (Arg463) and TtFRS (Arg321) which in its closed conformation clashes with this ring.

FIG. 7E is a close-up view of Compound 1 localized in the enzyme active site. Labelled residues show hydrophobic interactions with Compound 1.

FIG. 8A (left) shows the computed optimal conformation of Compound 1 in water.

FIG. 8A (right) shows the superimposed computed solution structure and crystallographic FRS-bound structure (green), with polar hydrogen atoms omitted for clarity-root mean square deviation: 1.1911 Å.

FIG. 8B shows the coordinates of Compound 1 which correlate with the optimized geometry and computed energy in Table 3.

FIG. 9A shows the sequence alignment of the a subunit of FRS from various eukaryotic pathogens including its human counterpart. Residues forming portions of the L-Phe pocket residues are in bold, single nucleotide polymorphisms (SNPs) identified for PfcFRS are in the black boxes, some non-conserved residues within 5 Å, which may responsible for selectivity, are in grey boxes with arrows and underlined are the residues, which are different in each species enzyme. Pf, Plasmodium falciparum: (SEQ ID NO: 24); Pv, Plasmodium vivax (SEQ ID NO: 25); Po, Plasmodium ovale (SEQ ID NO: 26); Pm, Plasmodium malariae (SEQ ID NO: 27), Hs, Homo sapiens (SEQ ID NO: 28); Crypto; Cryptosporidium hominis (SEQ ID NO: 29), Tg; Toxoplasma gondii (SEQ ID NO: 30).

FIG. 9B also shows the sequence alignment of 522 residues of the a subunit of FRS from various eukaryotic pathogens including its human counterpart. L-Phe site residues within 5 Å radius of Compound 1 are shown in the solid boxes, ATP site residues present with 5 Å radius of Compound 1 are circled, and Auxiliary site residues within 5 Å radius of Compound 1 are shown in the dashed boxes. In FIG. 9B, non-conserved residues in the binding pockets are highlighted in bold and underline. Pf, Plasmodium falciparum: (SEQ ID NO: 24); Pv, Plasmodium vivax (SEQ ID NO: 25); Po, Plasmodium ovale (SEQ ID NO: 26); Pm, Plasmodium malariae (SEQ ID NO: 27); Hs, Homo sapiens (SEQ ID NO: 31; residues 1-522 of SEQ ID NO: 28); Ch, Cryptosporidium hominis (SEQ ID NO: 32; residues 1-522 of SEQ ID NO: 29), Tg; Toxoplasma gondii (SEQ ID NO: 33; residues 1-522 of SEQ ID NO: 30).

FIG. 10A is a close-up view of PvcFRS (top labeled amino acid) with HscFRS (bottom labeled amino acid, PDB 3L4G) as superimposed structures. Non-conserved residues within 5 Å of Compound 1 are shown.

FIG. 10B depicts the PvcFRS structure with known PfcFRS mutation positions as disclosed in Kato et al., Nature 538 (2016): 344-349, which is hereby incorporated by reference in its entirety and particularly in relation to Supplementary Table 4, with each mutation labeled (M310, G506, L544, V539). All resistance mutations map within the confines of the active site region wherein the L550V (L544 in FIG. 10B) mutant lies closest to the bound ligand.

FIG. 11A illustrates the effect of L550V mutation on enzyme activity as illustrated by the Michaelis constant with L-Phe substrate. The error bars indicate standard deviation (n=3).

FIG. 11B illustrates the effect of L550V mutation on enzyme activity as illustrated by the Michaelis constant with ATP substrate. The error bars indicate standard deviation (n=3).

FIG. 12A shows the catalytic pocket of PvcFRS: Compound 1 complex superimposed with HscFRS-L-Phe (PDB 3L4G) and TtFRS-L-Phe-AMP-tRNA (PDB 2IY5). Compound 1 occupies L-Phe and auxiliary site (arrow) and its-methoxymethyl group (arrow) is proximal to ATP binding site. This variable site at the [6.2.0]-diazabicyclodecane base can be further manipulated to occupy ATP site.

FIG. 12B is a schematic map showing Compound 1 occupancy in the apicomplexan FRS polyppeptide. These discovered aaRS inhibitors operate via their multiple binding mechanisms at active site. The cFRS inhibitors of the present disclosure such as Compound 1 may occupy both the amino acid and auxiliary pockets.

### DETAILED DESCRIPTION OF THE INVENTION

The present disclosure provides compounds with unique MoA for inhibition of the FRS enzyme in apicomplexan parasites that do not inhibit or do not significantly inhibit the human ortholog of the Plasmodium phenylalanine tRNA-synthetase (cFRS) enzyme.

The invention is based, at least in part, on the discovery of a novel series of bicyclic azetidines that prevented malaria transmission, provided prophylaxis, and provided a single-dose cure in animal models of malaria. As reported in detail below, the present disclosure provides structural and biochemical evidence that bicyclic azetidines are competitive inhibitors of L-Phe, one of three substrates (along with ATP and tRNA) that function in the cFRS-catalyzed aminoacylation reaction that underpins protein synthesis in the parasite. Significantly, the disclosure provides the structural basis of cFRS inhibition via resolution of a co-crystal structure of the Plasmodium vivax enzyme (PvcFRS) bound to Compound 1. The inhibitor binds two distinct sub-sites within the PvcFRS catalytic site, occupying the L-Phe binding pocket along with an auxiliary cavity. Compound 1 traverses past the ATP binding site but does not fill it, consistent with the biochemical data supporting a novel mechanism of inhibition. Mutations known to confer resistance map to protein regions that cover the Compound 1 binding sites and result in significantly diminished enzymatic activity. Inhibitor selectivity is driven by distinct structural regions within parasite and human FRSs. Given that Compound 1 recognition residues are highly conserved amongst apicomplexan FRSs, this work lays the structural framework for the development of drugs against both Plasmodium and related apicomplexans.

### Bicyclic Azetidine Scaffolds

Compounds having a bicyclic azetidine scaffold exhibit multistage antimalarial activity and can achieve single-dose cures in a mouse model of malaria. These compounds exert their antimalarial activity via inhibition of Plasmodium phenylalanyl-tRNA synthetase (FRS), an enzyme essential for protein synthesis. Aminoacyl-tRNA synthetases (aaRS) activate amino acids as aminoacyl adenylates (AA-AMP) and enable their relay to the 3′-ends of cognate tRNAs as feed for ribosomes. Inhibition of aaRSs therefore results in the interruption of cell growth and ultimately in cell death.

Plasmodia have three protein translation compartments—in the cytoplasm, apicoplast, and mitochondria—where FRSs reside to feed charged tRNAs into ribosomal-based protein synthesis. In both P. falciparum and P. vivax, FRSs exist as heterodimers of alpha (a) and beta (p) subunits that further dimerize to form a complex of α2β2. This hetero-tetrameric organization of cytoplasmic phenylalanyl-tRNA synthetases (cFRSs) is conserved but with significant differences in the chain lengths and functions of α and β subunits. The FRS α subunit contains the active site and catalyzes the two-step aminoacylation reaction, while the main functions of the β subunit are to recognize the anticodon region of tRNA and to edit mischarged tRNA molecules with isosteric amino acids such as tyrosine. cFRSs are highly conserved and exhibit high sequence identity amongst Plasmodium species suggesting that FRS from all five parasites causative of human malaria can be targeted by a single chemical series.

However, a robust understanding of the requirements for such a chemical series is lacking. Identification of the most potent chemical candidates for FRS inhibition would be benefitted by such an understanding allowing for the creation or improvement of therapies for the treatment and/or prophylaxis of infections by species from the genus Plasmodium and other related apicomplexans, and diseases associated therewith (e.g., malaria).

### Compounds

The compounds of the disclosure provide increased inhibitory properties against apicomplexan parasite FRS enzymes due to the dual inhibitor structure identified herein. For example, the compound may have the structure of formula (I):

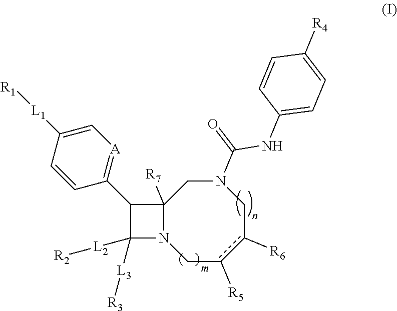

wherein the dashed bond (———) may be a single or double bond;
m is 0 (i.e., it is a bond) or 1;
n is 0, 1 or 2;
A is CH or N;
L₁ is absent, or —C≡C—;
L₂ and L₃ are independently absent, alkylene (e.g., C₁-C₄ alkylene, methylene), or heteroalkylene (e.g., C₁-C₄ heteroalkylene), wherein any of the foregoing groups optionally comprises one or more (e.g., one, two, three) points of substitution;
R₁ is hydrogen, aryl (e.g., C₅-C₁₂ aryl), or heteroaryl (e.g., C₅-C₁₂ heteroaryl), wherein any of the foregoing groups optionally comprises one or more (e.g., one, two, three) points of substitution;
R₂ and R₃ are independently hydrogen, —OH, —OR, —S(O)₂R, —N(R)S(O)₂R, —C(O)R, —OC(O)R, —N(R)C(O)R, —C(O)N(R)R, —N(R)₂, or heterocyclyl, and R₂ and/or R₃ has one or more (e.g., two, three, four, five) optional points of substitution;
R₄ is cycloalkoxy (e.g., C₃-C₆ cycloalkoxy, cyclopropoxy) optionally comprising one or more (e.g., one, two, three) points of substitution;
R₅ and R₆ are independently selected from hydrogen and —OH;
R₇ is hydrogen, —CH₂OH, or —CH₂OR;
R is independently selected at each occurrence from hydrogen and alkyl (e.g., C₁-C₁₂ alkyl, C₁-C₈ alkyl, C₁-C₅ alkyl, C₃-C₁₂ cycloalkyl), wherein each R has one or more (e.g., two, three, four, five) optional points of substitution (e.g., with OH, with C(O)OH, —CN, —NH₂, —N(R^{A})₂); and
R^{A} is independently selected at each occurrence from hydrogen and lower alkyl (e.g., C₁-C₄ alkyl, methyl, ethyl, propyl, isopropyl); or
pharmaceutically acceptable salts thereof; or
prodrugs of any of the foregoing;
wherein said compound is not

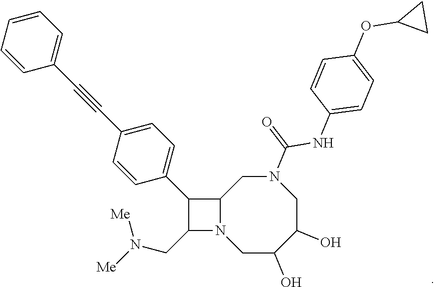 [SMILES: CN(C)CC1C(c2ccc(C#Cc3ccccc3)cc2)C2CN(C(=O)Nc3ccc(OC4CC4)cc3)CC(O)C(O)CN12]

In some embodiments, the compound may have the structure of formula (Ia), (Ib), or (Ic):

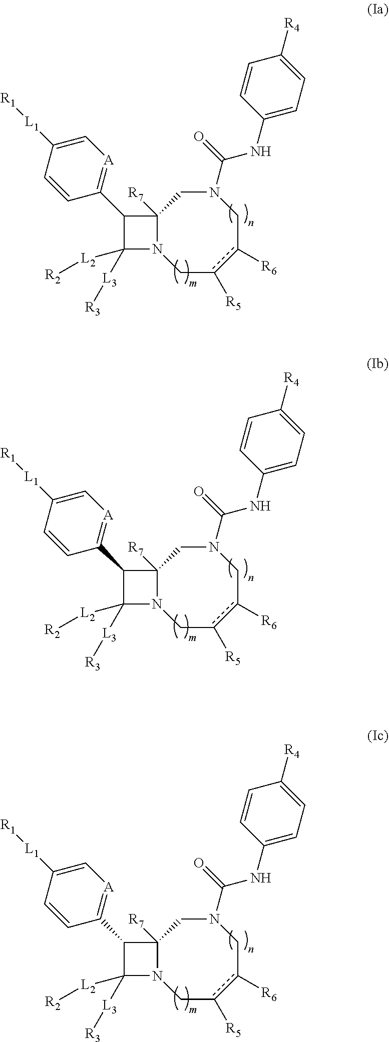

wherein the dashed bond (———) may be a single or double bond;
m is 0 (i.e., it is a bond) or 1;
n is 0, 1 or 2;
A is CH or N;
L₁ is absent, or —C≡C—;
L₂ and L₃ are independently absent, alkylene (e.g., C₁-C₄ alkylene, methylene), or heteroalkylene (e.g., C₁-C₄ heteroalkylene), wherein any of the foregoing groups optionally comprises one or more (e.g., one, two, three) points of substitution;
R₁ is hydrogen, aryl (e.g., C₅-C₁₂ aryl), or heteroaryl (e.g., C₅-C₁₂ heteroaryl), wherein any of the foregoing groups optionally comprises one or more (e.g., one, two, three) points of substitution;
R₂ and R₃ are independently hydrogen, —OH, —OR, —S(O)₂R, —N(R)S(O)₂R, —C(O)R, —OC(O)R, —N(R)C(O)R, —C(O)N(R)R, —N(R)₂, or heterocyclyl, and R₂ and/or R₃ has one or more (e.g., two, three, four, five) optional points of substitution;
R₄ is cycloalkoxy (e.g., C₃-C₆ cycloalkoxy, cyclopropoxy) optionally comprising one or more (e.g., one, two, three) points of substitution;
R₅ and R₆ are independently selected from hydrogen and OH;
R₇ is hydrogen, CH₂OH, or CH₂OR;
R is independently selected at each occurrence from hydrogen and alkyl (e.g., C₁-C₁₂ alkyl, C₁-C₈ alkyl, C₁-C₅ alkyl, C₃-C₁₂ cycloalkyl), wherein each R has one or more (e.g., two, three, four, five) optional points of substitution (e.g., with OH, with C(O)OH, —CN, —NH₂, —N(R^{A})₂); and
R^{A} is independently selected at each occurrence from hydrogen and lower alkyl (e.g., C₁-C₄ alkyl, methyl, ethyl, propyl, isopropyl); or
pharmaceutically acceptable salts thereof; or
prodrugs of any of the foregoing;
wherein said compound is not

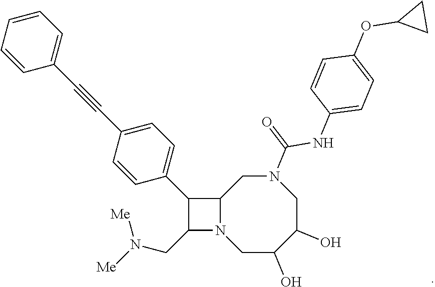 [SMILES: CN(C)CC1C(c2ccc(C#Cc3ccccc3)cc2)C2CN(C(=O)Nc3ccc(OC4CC4)cc3)CC(O)C(O)CN12]

In various embodiments, the compound having the structure of Formula (I) is not a compound disclosed in Table 1 of WO 2018175385 (e.g., compound E1 ((3S,4R,8R,9S,10S)—N-(4-cyclopropoxyphenyl)-10-((dimethylamino)methyl)-3,4-dihydroxy-9-(4-(phenylethynyl) phenyl)-1,6-diazabicyclo[6.2.0]decane-6-carboxamide), compound E38 ((3R,4S,8R,9S,10S)—N-(4-cyclopropoxyphenyl)-10-((dimethylamino)methyl)-3,4-dihydroxy-9-(4-(phenylethynyl) phenyl)-1,6-diazabicyclo[6.2.0]decane-6-carboxamide)) or Table 1 of US 2016/0289235, which are hereby incorporated by reference in their entirety.

As shown herein, the compounds of the present disclosure provide substantial biological activity against apicomplexan parasites, such as those from the genus Plasmodium (e.g., Plasmodium falciparum (Pj), Plasmodium vivax (Pv), Plasmodium ovale (Po), Plasmodium malariae (Pm), Plasmodium fragile (Pfr), Plasmodium inui (Pi), Plasmodium gonderi (Pg)), Cryptosporidium (e.g., Cryptosporidium parvum (Cp), Cryptosporidium hominis (Ch)), or Toxoplasma (e.g., Toxoplasma gondii (Tg)). In various implementations, the compound has a Plasmodium falciparum: Dd2 growth inhibition EC₅₀ of less than 1 nM. In some embodiments, the compound has an apicomplexan FRS dissociation constant (e.g., PfcFRS EC50, PvcFRS EC50) of less than 50 nM or less than 25 nM or less than 10 nM. In some embodiments, the compound has a human cFRS dissociation constant of more than 100 nM or more than 1000 nM or more than 10000 nM. In some embodiments, the compound has an apicomplexan cFRS dissociation constant (e.g., PfcFRS EC50, PvcFRS EC50) of less than 50 nM (e.g., less than 25 nM, less than 10 nM) and a human cFRS dissociation constant of more than 100 nM (e.g., more than 1000 nM, or more than 10000 nM). In various implementations, the compound may have an IC50 for cFRS of less than 100 nM or less than 50 nM or less than 30 nM.

Typically, alkyl groups described herein refer to a branched or straight-chain monovalent saturated aliphatic hydrocarbon radical of 1-30 carbon atoms (e.g., 1-16 carbon atoms, 6-20 carbon atoms, 8-16 carbon atoms, or 4-18 carbon atoms, 4-12 carbon atoms). In some embodiments, the alkyl group may be substituted with 1, 2, 3, or 4 substituent groups as defined herein. Alkyl groups may have from 1-26 carbon atoms. In other embodiments, alkyl groups will have from 6-18 or from 1-8 or from 1-6 or from 1-4 or from 1-3 carbon atoms, including for example, embodiments having one, two, three, four, five, six, seven, eight, nine, or ten carbon atoms. Any alkyl group may be substituted or unsubstituted. Examples of alkyl groups include methyl, ethyl, propyl, butyl, pentyl, hexyl, heptyl, octyl, nonyl, decyl, undecyl, and dodecyl groups. Heteroalkyl groups may refer to branched or straight-chain monovalent saturated aliphatic hydrocarbon radicals with one or more heteroatoms (e.g., N, O, S) in the carbon chain. Heteroalkyl groups may have 1-30 carbon atoms (e.g., 1-16 carbon atoms, 6-20 carbon atoms, 8-16 carbon atoms, or 4-18 carbon atoms, 4-12 carbon atoms). In some embodiments, the heteroalkyl group may be substituted with 1, 2, 3, or 4 substituent groups as defined herein. Heteroalkyl groups may have from 1-26 carbon atoms. In other embodiments, heteroalkyl groups will have from 6-18 or from 1-8 or from 1-6 or from 1-4 or from 1-3 carbon atoms, including for example, embodiments having one, two, three, four, five, six, seven, eight, nine, or ten carbon atoms. In some embodiments, the heteroalkyl group can be further substituted with 1, 2, 3, or 4 substituent groups as described herein for alkyl groups. Examples of heteroalkyl groups are an alkoxy. Alkoxy substituent groups or alkoxy-containing substituent groups may be substituted by, for example, one or more alkyl groups.

Aryl groups may be aromatic mono- or polycyclic radicals of 6 to 12 carbon atoms having at least one aromatic ring. Examples of such groups include, but are not limited to, phenyl, naphthyl, 1,2,3,4-tetrahydronaphthalyl, 1,2-dihydronaphthalyl, indanyl, and 1H-indenyl. Typically, heteroaryls include mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. Examples of heteroaryl groups are pyridyl, benzooxazolyl, benzoimidazolyl, and benzothiazolyl.

A substituted hydrocarbon group may have as a substituent one or more hydrocarbon radicals, substituted hydrocarbon radicals, or may comprise one or more heteroatoms. Examples of substituted hydrocarbon radicals include, without limitation, heterocycles, such as heteroaryls. Unless otherwise specified, a hydrocarbon substituted with one or more heteroatoms will comprise from 1-20 heteroatoms. In other embodiments, a hydrocarbon substituted with one or more heteroatoms will comprise from 1-12 or from 1-8 or from 1-6 or from 1-4 or from 1-3 or from 1-2 heteroatoms. Examples of heteroatoms include, but are not limited to, oxygen, nitrogen, sulfur, phosphorous, halogen (e.g., F, Cl, Br, I), boron, or silicon. In some embodiments, heteroatoms will be selected from the group consisting of oxygen, nitrogen, sulfur, phosphorous, and halogen (e.g., F, Cl, Br, I). In some embodiments, a heteroatom or group may substitute a carbon (e.g., substituted alkyl may include heteroalkyl). In some embodiments, a heteroatom or group may substitute a hydrogen. In some embodiments, a substituted hydrocarbon may comprise one or more heteroatoms in the backbone or chain of the molecule (e.g., interposed between two carbon atoms, as in “oxa”). In some embodiments, a substituted hydrocarbon may comprise one or more heteroatoms pendant from the backbone or chain of the molecule (e.g., covalently bound to a carbon atom in the chain or backbone, as in “oxo”).

Unless otherwise noted, all groups described herein (e.g., alkyl, cycloalkyl, heteroalkyl, heterocycloalkyl, aryl, heteroaryl, alkylene, heteroalkylene, cylcoalkylene, heterocycloalkylene) may optionally contain one or more substituents, to the extent permitted by valency. Common substituents include halogen (e.g., F, Cl), C₁₋₁₂ straight chain or branched chain alkyl, C₂₋₁₂ alkenyl, C₂₋₁₂ alkynyl, C₃₋₁₂ cycloalkyl, C₆₋₁₂ aryl, C₃₋₁₂ heteroaryl, C₃₋₁₂heterocyclyl, C₁₋₁₂ alkylsulfonyl, nitro, cyano, —COOR, —C(O)NRR′, —OR, —SR, —NRR′, and oxo, such as mono- or di- or tri-substitutions with moieties such as halogen, fluoroalkyl, perfluoroalkyl, perfluroalkoxy, trifluoromethoxy, chlorine, bromine, fluorine, methyl, methoxy, pyridyl, furyl, triazyl, piperazinyl, pyrazoyl, imidazoyl, and the like, each optionally containing one or more heteroatoms such as halo, N, O, S, and P. R and R′ are independently hydrogen, C₁₋₁₂ alkyl, C₁₋₁₂ haloalkyl, C₂₋₁₂ alkenyl, C₂₋₁₂ alkynyl, C₃₋₁₂cycloalkyl, C₄₋₂₄ cycloalkylalkyl, C₆₋₁₂ aryl, C₇₋₂₄ aralkyl, C₃₋₁₂ heterocyclyl, C₃₋₂₄ heterocyclylalkyl, C₃₋₁₂ heteroaryl, or C₄₋₂₄ heteroarylalkyl. Further, as used herein, the phrase optionally substituted indicates the designated hydrocarbon group may be unsubstituted (e.g., substituted with H) or substituted. Typically, substituted hydrocarbons are hydrocarbons with a hydrogen atom removed and replaced by a substituent (e.g., a common substituent).

It is understood by one of ordinary skill in the chemistry art that substitution at a given atom is limited by valency. The use of a substituent (radical) prefix names such as alkyl without the modifier optionally substituted or substituted is understood to mean that the particular substituent is unsubstituted. However, the use of haloalkyl without the modifier optionally substituted or substituted is still understood to mean an alkyl group, in which at least one hydrogen atom is replaced by halo. Where a group may be substituted by one or more of a number of substituents, such substitutions are selected so as to comply with principles of chemical bonding with regard to valencies, and to give compounds which are not inherently unstable. For example, any carbon atom will be bonded to two, three, or four other atoms, consistent with the four valence electrons of carbon. Additionally, when a structure has less than the required number of functional groups indicated, those carbon atoms without an indicated functional group are bonded to the requisite number of hydrogen atoms to satisfy the valency of that carbon.

Compounds provided herein can have one or more asymmetric carbon atoms and can exist in the form of optically pure enantiomers, mixtures of enantiomers such as racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms including stereoisomers, enantiomers, diastereomers, or racemates (i.e., the compound exists as a mixture containing two enantiomers and does not rotate polarized light). Enantiomers of a compound can be prepared, for example, by separating an enantiomer from a racemate using one or more well-known techniques and methods, such as chiral chromatography and separation methods based thereon. The appropriate technique and/or method for separating an enantiomer of a compound described herein from a racemic mixture can be readily determined by those of skill in the art.

The compound provided herein may also be present as geometric isomer which differ in the orientation of substituent atoms (e.g., to a carbon-carbon double bond, to a cycloalkyl ring, to a bridged bicyclic system). Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule and may be used to indicate the geometric configuration of the presently disclosed compounds. Certain of the disclosed compounds may exist in atropisomeric forms. Atropisomers are stereoisomers resulting from hindered rotation about single bonds where the steric strain barrier to rotation is high enough to allow for the isolation of the conformers.

The compounds disclosed herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer may be typically more than 50% (e.g., at least 55%, 60%, 70%, 80%, 90%, 99%, or 99.9%) by weight (or mole fraction) relative to the other stereoisomers. When a single enantiomer is named or depicted by structure, the depicted or named enantiomer is more than 50% (e.g., at least 55%, 60%, 70%, 80%, 90%, 99%, or 99.9%) by weight (or mole fraction) optically pure. When a single diastereomer is named or depicted by structure, the depicted or named diastereomer is more than 50% (e.g., at least 55%, 60%, 70%, 80%, 90%, 99%, or 99.9%) by weight (or mole fraction) pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The disclosure embraces all of these forms.

Solvates of the compounds described herein may form the aggregate of the compound or an ion of the compound with one or more solvents. Such solvents may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The compounds described herein may be present as a pharmaceutically acceptable salt. Typically, salts are composed of a related number of cations and anions (at least one of which is formed from the compounds described herein) coupled together (e.g., the pairs may be bonded ionically) such that the salt is electrically neutral. Pharmaceutically acceptable salts may retain or have similar activity to the parent compound (e.g., an ED50 within 10%) and have a toxicity profile within a range that affords utility in pharmaceutical compositions. For example, pharmaceutically acceptable salts may be suitable for use in contact with the tissues of humans and animals without undue toxicity, irritation, allergic response and are commensurate with a reasonable benefit/risk ratio. Pharmaceutically acceptable salts are described in: Berge et al., J Pharmaceutical Sciences 66:1-19, 1977 and in Pharmaceutical Salts: Properties, Selection, and Use, (Eds. P. H. Stahl and C. G. Wermuth), Wiley-VCH, 2008. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, dichloroacetate, digluconate, dodecylsulfate, ethanesulfonate, formate, fumarate, glucoheptonate, glutamate, glycerophosphate, hemisulfate, heptonate, hexanoate, hippurate, hydrobromide, hydrochloride, hydroiodide, 2-hydroxy-ethanesulfonate, isethionate, lactobionate, lactate, laurate, lauryl sulfate, malate, maleate, malonate, mandelate, methanesulfonate, mucate, 2-naphthalenesulfonate, nicotinate, nitrate, oleate, oxalate, palmitate, pamoate, pantothenate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative basic salts include alkali or alkaline earth metal salts include sodium, lithium, potassium, calcium, and magnesium, aluminum salts, as well as nontoxic ammonium, quaternary ammonium, and amine cations, including, but not limited to ammonium, tetramethylammonium, tetraethylammonium, methylamine, dimethylamine, trimethylamine, triethylamine, caffeine, and ethylamine.

Pharmaceutically acceptable acid addition salts of the disclosure can be formed by the reaction of a compound of the disclosure with an equimolar or excess amount of acid. Alternatively, hemi-salts can be formed by the reaction of a compound of the disclosure with the desired acid in a 2:1 ratio, compound to acid. The reactants are generally combined in a mutual solvent such as diethyl ether, tetrahydrofuran, methanol, ethanol, iso-propanol, benzene, or the like. The salts normally precipitate out of solution within, e.g., one hour to ten days and can be isolated by filtration or other conventional methods.

The present disclosure identifies the potential dual inhibiting mechanism of action for these compounds which may bind to two or more binding pockets in the apicomplexan FRS polypeptide. Compounds may be prepared to utilize the dual inhibitory nature afforded by the bicyclic azetidine scaffold due in part to its rigidity and nonplanarity. Specific functionalization at the outermost groups of the bicyclic azetidines, and particularly those groups implicated in binding, will affect the resultant inhibitory effect. For example, the compound may have the structure of formula (II):

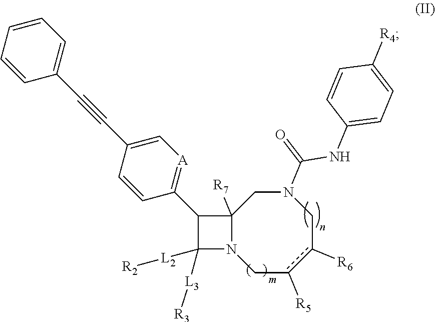

pharmaceutically acceptable salts thereof; or
prodrugs of any of the foregoing.

In some embodiments, the compound may have the structure of formula (III):

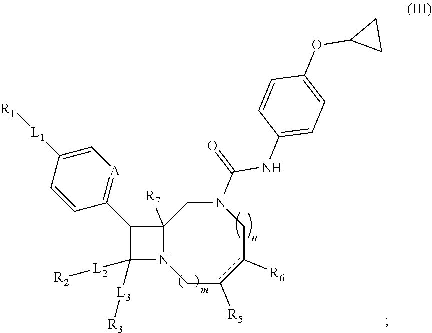

pharmaceutically acceptable salts thereof; or
prodrugs of any of the foregoing.

In particular, the compound may be:

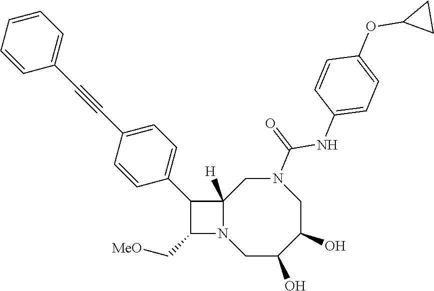 [SMILES: COC[C@@H]1C(c2ccc(C#Cc3ccccc3)cc2)[C@@H]2CN(C(=O)Nc3ccc(OC4CC4)cc3)C[C@@H](O)[C@@H](O)CN12]

((3S,4R,8R,10S)—N-(4-cyclopropoxyphenyl)-3,4-dihydroxy-10-(methoxymethyl)-9-(4-(phenylethynyl)phenyl)-1,6-diazabicyclo[6.2.0]decane-6-carboxamide) or pharmaceutically acceptable salts thereof, or prodrugs of any of the foregoing. For example, in some embodiments, the compound has the structure of:

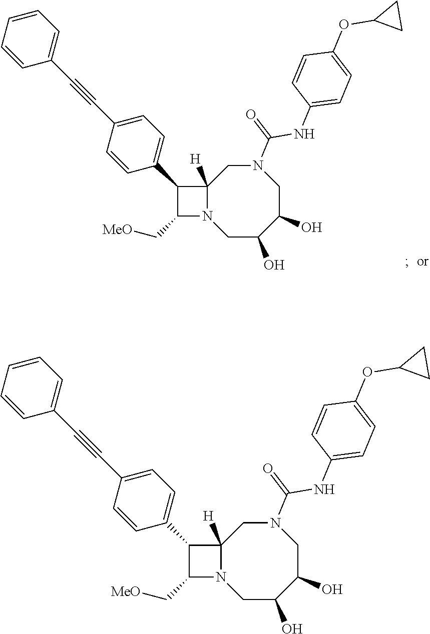 [SMILES: COC[C@@H]1[C@@H](c2ccc(C#Cc3ccccc3)cc2)[C@@H]2CN(C(=O)Nc3ccc(OC4CC4)cc3)C[C@@H](O)[C@@H](O)CN12 ; COC[C@@H]1[C@H](c2ccc(C#Cc3ccccc3)cc2)[C@@H]2CN(C(=O)Nc3ccc(OC4CC4)cc3)C[C@@H](O)[C@@H](O)CN12]

or is a racemic mixture thereof. In some embodiments, the compound is compound 1 having the structure:

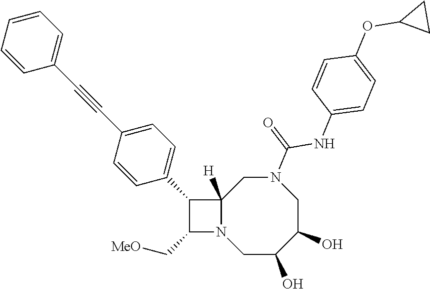 [SMILES: COC[C@@H]1[C@H](c2ccc(C#Cc3ccccc3)cc2)[C@@H]2CN(C(=O)Nc3ccc(OC4CC4)cc3)C[C@@H](O)[C@@H](O)CN12]

or pharmaceutically acceptable salts thereof, or prodrugs of any of the foregoing. It will be understood that in the event of any inconsistency between a chemical name and formula, both compounds with the indicated chemical name and compounds with the indicated chemical structure will be considered as embraced by the invention.

The compounds of the present invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include C₁₋₆ alkyl esters of carboxylic acid groups, which, upon administration to a subject, are capable of providing active compounds.

### Pharmaceutical Compositions

The compounds described herein (e.g., compounds having the structure of, Formula (I), Formula (II), Formula (III), Formula (IV), Compound 1, and compounds identified with the screening assays described herein) are useful for the treatment of an apicomplexan parasitic disease (e.g., a disease caused by a parasite from the genus Plasmodium, such as malaria, Toxoplasma, such as toxoplasmosis, or Cryptosporidium such as cryptosporidiosis) in a subject in need thereof. The compounds described herein may also be compounds for use in the preparation of a medicament for the treatment of an apicomplexan parasitic disease (e.g., a disease caused by a parasite from the genus Plasmodium, such as malaria, Toxoplasma, such as toxoplasmosis, or Cryptosporidium such as cryptosporidiosis) in a subject in need thereof. Due to similarities in the FRS structures of these apicomplexan species, the compounds identified herein are effective for treatment of multiple diseases associated with apicomplexan infection, such as two or more of malaria, toxoplasmosis, and cryptosporidiosis.

Pharmaceutical dosage forms are provided as well, which may comprise a compound of the present disclosure (e.g., compounds having the structure of Formula (I), Formula (II), Formula (III), Formula (IV), Compound 1, compounds identified with the screening assays disclosed herein) and one or more pharmaceutically acceptable carriers, diluents, or excipients.

Unit dosage forms, also referred to as unitary dosage forms, often denote those forms of medication supplied in a manner that does not require further weighing or measuring to provide the dosage (e.g., tablet, capsule, caplet). The compositions of the present disclosure may be present as unit dosage forms. For example, a unit dosage form may refer to a physically discrete unit suitable as a unitary dosage for human subjects and other species, each unit containing a predetermined quantity of active material calculated to produce the desired therapeutic effect, in association with any suitable pharmaceutical excipient or excipients. Exemplary, non-limiting unit dosage forms include a tablet (e.g., a chewable tablet), caplet, capsule (e.g., a hard capsule or a soft capsule), lozenge, film, strip, and gel cap. In certain embodiments, the compounds described herein, including crystallized forms, polymorphs, and solvates thereof, may be present in a unit dosage form.

Useful pharmaceutical carriers, excipients, and diluents for the preparation of the compositions hereof, can be solids, liquids, or gases. These include any and all solvents, dispersion media, coatings, antibacterial and antifungal agents, isotonic and absorption delaying agents and the like. The pharmaceutically acceptable carrier or excipient does not destroy the pharmacological activity of the disclosed compound and is nontoxic when administered in doses sufficient to deliver a therapeutic amount of the compound. Thus, the compositions can take the form of tablets, pills, capsules, suppositories, powders, enterically coated or other protected formulations (e.g., binding on ion-exchange resins or packaging in lipid-protein vesicles), sustained release formulations, solutions, suspensions, elixirs, and aerosols. The carrier can be selected from the various oils including those of petroleum, animal, vegetable or synthetic origin, e.g., peanut oil, soybean oil, mineral oil, and sesame oil. Water, saline, aqueous dextrose, and glycols are examples of liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution and rendering the solution sterile. Suitable pharmaceutical excipients include starch, cellulose, chitosan, talc, glucose, lactose, gelatin, malt, rice, flour, chalk, silica, magnesium stearate, sodium stearate, glycerol monostearate, sodium chloride, dried skim milk, glycerol, propylene glycol, water, and ethanol. The compositions may be subjected to conventional pharmaceutical additives such as preservatives, stabilizing agents, wetting or emulsifying agents, salts for adjusting osmotic pressure, and buffers. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for administration to the recipient.

Non-limiting examples of pharmaceutically acceptable carriers and excipients include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self-emulsifying drug delivery systems (SEDDS) such as d-atocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers. Cyclodextrins such as α-, β-, and γ-cyclodextrin, or chemically modified derivatives such as hydroxyalkylcyclodextrins, including 2- and 3-hydroxypropyl-cyclodextrins, or other solubilized derivatives can also be used to enhance delivery of the compounds described herein.

The pharmaceutical composition may also be formulated as a veterinary composition, intended for use with subjects other than humans. The veterinary compositions according to the present invention can be in any appropriate forms to suit the requested administration modes, for instance nasal, oral, intradermic, cutaneous or parenteral. In a certain embodiment, the composition is in a form intended for an oral administration and, for instance when the domestic animal eating, either mixed to the food ration, or directly into the mouth after meal. The veterinary compositions of the invention are in the form of a nasal, oral or injectable liquid suspension or solution, or in solid or semi-solid form, powders, pellets, capsules, granules, sugar-coated pills, gelules, sprays, cachets, pills, tablets, pastes, implants or gels. In a particular embodiment, the compositions are in the form of an oral solid form including tablets. In some embodiments, the veterinary compositions may have an effective amount of the compound for a specific species of animal (e.g., cow, lamb, goat, horse).

In various embodiments, the compositions of the invention are formulated in pellets or tablets for an oral administration. According to this type of formulation, they comprise lactose monohydrate, cellulose microcrystalline, crospovidone/povidone, aroma, compressible sugar and magnesium stearate as excipients. When the compositions are in the form of pellets or tablets, they are for instance 1 mg, 2 mg, or 4 mg pellets or tablets. Such pellets or tablets are divisible so that they can be cut to suit the posology according to the invention in one or two daily takes. In a further embodiment, the compositions of the disclosure are formulated in injectable solutions or suspensions for a parenteral administration. The injectable compositions are produced by mixing therapeutically efficient quantity of torasemide with a pH regulator, a buffer agent, a suspension agent, a solubilization agent, a stabilizer, a tonicity agent and/or a preservative, and by transformation of the mixture into an intravenous, sub-cutaneous, intramuscular injection or perfusion according to a conventional method. Possibly, the injectable compositions may be lyophilized according to a conventional method. Examples of suspension agents include methylcellulose, polysorbate 80, hydroxyethylcellulose, xanthan gum, sodic carboxymethylcellulose and polyethoxylated sorbitan monolaurate. Examples of solubilization agent include polyoxy ethylene-solidified castor oil, polysorbate 80, nicotinamide, polyethoxylated sorbitan monolaurate, macrogol and ethyl ester of caste oil fatty acid. Moreover, the stabilizer includes sodium sulfite, sodium metalsulfite and ether, while the preservative includes methyl p-hydroxybenzoate, ethyl p-hydroxybenzoate, sorbic acid, phenol, cresol and chlorocresol. An example of tonicity agent is mannitol. When preparing injectable suspensions or solutions, it is desirable to make sure that they are blood isotonic.

In some embodiments, the pharmaceutical composition further comprises a viscosity enhancing agent. In some embodiments, the viscosity enhancing agent includes methylcellulose, hydroxyethylcellulose, hydroxypropylmethylcellulose and smart hydrogel. In some embodiments, the viscosity enhancing agent is hydroxyethylcellulose. In some embodiments, the pharmaceutical composition comprises 0.01-1.0% (w/v) viscosity enhancing agent. In other embodiments, the intranasal pharmaceutical composition comprises 0.05% (w/v) hydroxyethylcellulose.

In some embodiments, the pH of the pharmaceutical composition is from 4.0 to 7.5. In other embodiments, the pH of the pharmaceutical composition is from 4.0 to 6.5. In another embodiment the pharmaceutical composition has a pH of from 5.5 to 6.5. In further embodiments, the pharmaceutical composition has a pH of from 6.0 to 6.5. In various implementations, the pH of said aqueous solution or liquid formulation is from pH 3 to pH 7, from pH 3 to pH 6, from pH 4 to pH 6, or from pH 5 to pH 6. These pH ranges may be achieved through the incorporation of one or more pH modifying agents, buffers, and the like. In some embodiments, a pH modifier such as acetic acid, is present in a final concentration of at least 0.001%, preferably at least 0.01%, more preferably between 0.01%-0.2% by weight of the composition.

In terms of their form, compositions of this invention may include solutions, emulsions (including microemulsions), suspensions, creams, lotions, gels, powders, or other typical solid or liquid compositions used for application to skin and other tissues where the compositions may be used. Such compositions may contain: additional antimicrobials, moisturizers and hydration agents, penetration agents, preservatives, emulsifiers, natural or synthetic oils, solvents, surfactants, detergents, gelling agents, emollients, antioxidants, fragrances, fillers, thickeners, waxes, odor absorbers, dyestuffs, coloring agents, powders, viscosity-controlling agents and water, and optionally including anesthetics, anti-itch actives, botanical extracts, conditioning agents, darkening or lightening agents, glitter, humectants, mica, minerals, polyphenols, silicones or derivatives thereof, sunblocks, vitamins, and phytomedicinals. In certain embodiments, the composition of the invention is formulated with the above ingredients so as to be stable for a long period of time, as may be beneficial where continual or long-term treatment is intended.

### Methods for Parasite FRS Inhibition

Typically, the treatment of a disease, disorder, or condition (e.g., the conditions described herein such as those associated with infection) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. A disease, disorder, or condition may be palliated which includes that the extent and/or undesirable clinical manifestations of the disease, disorder, or condition are lessened and/or time course of the progression is slowed or lengthened, as compared to the extent or time course in the absence of treatment.

The method of prophylaxis or treatment of an apicomplexan parasitic disease (including diseases associated with apicomplexan parasite infection) of a subject in need thereof may comprise administration to the subject a compound (e.g., compounds having the structure of Formula (I), Formula (II), Formula (III), Formula (IV), Compound 1, compounds identified from the screening assays described herein) or composition of the present disclosure. In some embodiments, the apicomplexan parasite is from the genus Plasmodium (e.g., Plasmodium falciparum (Pj), Plasmodium vivax (Pv), Plasmodium ovale (Po), Plasmodium malariae (Pm), Plasmodium fragile (Pfr), Plasmodium inui (Pi), Plasmodium gonderi (Pg)), Cryptosporidium (e.g., Cryptosporidium parvum (Cp), Cryptosporidium hominis (Ch)), or Toxoplasma (e.g., Toxoplasma gondii (Tg)). In some embodiments, the disease, disorder, or condition associated with parasite infection is malaria, cryptosporidiosis, or toxoplasmosis.

Methods for preventing the growth of a population of apicomplexan parasites in a medium are also provided which may comprise contacting the medium with a compound of the present disclosure (e.g., compounds having the structure of Formula (I), Formula (II), Formula (III), Formula (IV), Compound 1, inhibitor compounds identified from the screening assays described herein). In some embodiments, the apicomplexan parasite is from the genus Plasmodium (e.g., Plasmodium falciparum (Pj), Plasmodium vivax (Pv), Plasmodium ovale (Po), Plasmodium malariae (Pm), Plasmodium fragile (Pfr), Plasmodium inui (Pi), Plasmodium gonderi (Pg)), Cryptosporidium (e.g., Cryptosporidium parvum (Cp), Cryptosporidium hominis (Ch)), or Toxoplasma (e.g., Toxoplasma gondii (Tg)). In some embodiments, the disease, disorder, or condition associated with parasite infection may be malaria, cryptosporidiosis, or toxoplasmosis.

In order to treat, prevent, or prevent recurrence of diseases, disorders, or conditions (e.g., malaria, cryptosporidiosis, or toxoplasmosis) as discussed herein, the compounds or compositions of the present disclosure may be administered at least once a day for at least one week. In various embodiments, the composition is administered at least twice a day for at least two days. In certain embodiments, the composition is administered approximately daily, at least daily, twice a week, weekly, or for once a month. In certain embodiments, the composition of the invention is administered for several months, such as at least two months, six months, or one year or longer. The invention is further suited for long-term use, which may be particularly beneficial for preventing recurring infection, or for preventing infection or conditions in at-risk or susceptible patients, including immune compromised patients. Such long-term use may involve treatment for at least two years, three years, four years, or even five or more years.

The compounds and pharmaceutical compositions can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

Examples of other drugs to combine with the compounds described herein include pharmaceuticals for the treatment of malaria (e.g., quinoline derivatives, aminoquinoline derivatives, mefloquine, chloroquine, hydroxychloroquine, doxycycline, atovaquone/proguanil, cladosporin class, halofuginone), cryptosporidiosis (e.g., nitazoxanide, paromomycin), or toxoplasmosis (e.g., pyrimethamine, sulfadiazine). Other examples of drugs to combine with the compounds described herein include pharmaceuticals for the treatment of different, yet associated or related symptoms or indications (e.g., folinic acid, clindamycin). Combination methods can involve the use of the two (or more) agents formulated together or separately, as determined to be appropriate. In one example, two or more drugs are formulated together for the simultaneous or near simultaneous administration of the agents.

### Kits

In another aspect, the composition of the invention is a kit, which contains the compositions of the present disclosure packaged to facilitate dispensing and/or administration of the compositions disclosed herein. The packaging or dispenser may include a bottle, tube, spray bottle, or other dispenser. In certain embodiments of the invention, the composition is packaged in a concentrated form, and diluted to a desired concentration upon use by the end user. Typically, in these aspects, the composition may be formulated and packaged in a manner suitable for long-term storage to maintain efficacy of the composition.

### Screening Methods and Systems

The present disclosure is based, in part, on the crystallization of an exemplary apicomplexan FRS enzyme with a compound. Armed with this crystallization, one of skill in the art can extrapolate to how other compounds would interact with other apicomplexan cFRS (e.g., PcFRS, CcFRS, TcFRS, PvcFRS, PfcFRS, PmcFRS, PocFRS, CpcFRS, TgcFRS). Compounds identified from the screening assays described herein are provided in the present disclosure. In some embodiments, the enzyme is cytoplasmic phenylalanyl-tRNA synthetase enzyme from a species in the Plasmodium genus (PcFRS), such as Plasmodium falciparum cytoplasmic phenylalanyl-tRNA synthetase (PfcFRS) or Plasmodium vivax cytoplasmic phenylalanyl-tRNA synthetase (PvcFRS). In various implementations, the crystalline form is characterized by the Protein Data Bank Structure 7BY6, which is hereby incorporated by reference in its entirety. The crystalline form may diffract to a resolution of from 2 to 4 Å (e.g., from 2.5 to 3.5 Å, from 3.9 to 4.1 Å).

These crystalline forms (and the atomic coordinates associated therewith) are useful for screening drug candidates for apicomplexan parasite infection. Methods for identifying an agent that binds to a binding pocket of a phenylalanyl-tRNA synthetase enzyme from an apicomplexan species (e.g., apicomplexan cFRS, PcFRS, CcFRS, TcFRS, PvcFRS, PfcFRS, PmcFRS, PocFRS, CpcFRS, TgcFRS) or a fragment thereof are provided which may comprise:
(a) performing a computational fitting operation between a candidate agent and a three-dimensional representation of the apicomplexan enzyme; and
(b) quantifying an association between the candidate agent and the three-dimensional representation of the L-Phe binding site of the apicomplexan enzyme and/or the ATP binding site of the apicomplexan enzyme, thereby identifying an agent that binds to a binding pocket of the apicomplexan enzyme or a fragment thereof.

The method for identifying an agent that binds to a binding pocket of a phenylalanyl-tRNA synthetase enzyme from an apicomplexan species (e.g., apicomplexan cFRS, PcFRS, CcFRS, TcFRS, PvcFRS, PfcFRS, PmcFRS, PocFRS, CpcFRS, TgcFRS) or a fragment thereof may comprise:
(a) generating a three-dimensional representation of a binding pocket of apicomplexan enzyme or a fragment thereof from an X-ray crystal structure of one or more binding pockets of the apicomplexan enzyme or a fragment thereof;
(b) identifying the amino acids of the binding pocket of apicomplexan enzyme or a fragment thereof;
(c) generating a three-dimensional representation of the apicomplexan enzyme or a fragment thereof;
(d) performing a computational fitting operation between a candidate agent and the three-dimensional representation of the apicomplexan vivax enzyme or a fragment thereof; and
(e) quantifying an association between the candidate agent and the three-dimensional representation of the apicomplexan enzyme or a fragment thereof, thereby identifying an agent that binds to apicomplexan enzyme or a fragment thereof (e.g., with a large enough association therebetween).

The screening methods may use apicomplexan FRS enzymes from a species in the Plasmodium genus (PcFRS) such as Plasmodium falciparum: cytoplasmic phenylalanyl-tRNA synthetase (PfcFRS) or Plasmodium vivax cytoplasmic phenylalanyl-tRNA synthetase (PvcFRS). In certain embodiments, the apicomplexan FRS enzyme is a cytoplasmic FRS enzyme (cFRS). In some embodiments, the binding pockets of these screening methods may comprise one or more of an amino acid sequence selected from amino acids 443-552 of Plasmodium vivax enzyme. For example, the binding pocket may comprise any one or more of the following amino acids: Arg443, Glu445, Val458, His451, Phe455, Gln457, Glu459, Tyr480, I1e483, Tyr 497, Gly506, His508, Glu510, Lys512, Lys513, Leu515, Val517, Asn519, Ala541, Trp542, Gly543, Leu544, Pro549, and I1e552 of PvcFRS or corresponding amino acids of other apicomplexan polypeptides.

In another embodiment, the disclosure provides a machine-readable storage medium which comprises the structural coordinates of an apicomplexan polypeptide (e.g., PfcFRS, PvcFRS, mutant PfcFRS, mutant PvcFRS) or fragments thereof including one or more of the binding sites identified herein. A storage medium encoded with these data is typically capable of displaying a three-dimensional graphical representation of a molecule or molecular complex which comprises such binding sites on a computer screen or similar viewing device. A compound may be considered to bind to a polypeptide (e.g., apicomplexan cFRS) if the compound has a physicochemical affinity for that polypeptide.

The invention also provides methods for designing, evaluating and identifying compounds that bind to the binding sites of FRS in apicomplexan species. Such compounds are expected to inhibit parasitic biological activity including parasite reproduction. The disclosure provides a system or computer for producing a) a three-dimensional representation of a molecule or molecular complex with an apicomplexan FRS polypeptide (e.g., apicomplexan cFRS, PcFRS, CcFRS, TcFRS, PvcFRS, PfcFRS, PmcFRS, PocFRS, CpcFRS, TgcFRS), wherein the molecule; or b) a three-dimensional representation of a homologue of said molecule or molecular complex, wherein said computer may comprise:
(i) a machine-readable data storage medium comprising a data storage material encoded with machine-readable data comprising the structure coordinates of amino acid residues in the FRS binding site;
(ii) a working memory for storing instructions for processing said machine-readable data;
(iii) a central-processing unit coupled to the working memory and to the machine-readable data storage medium for processing said machine readable data into the three-dimensional representation; and
(iv) optionally a display coupled to the central-processing unit for displaying said three-dimensional representation.

These systems may produce a three-dimensional graphical structure of a molecule or a molecular complex of the binding. Typically, computer modelling is the application of a computational program to determine one or more of the following: the location and binding proximity of a ligand to a binding moiety, the occupied space of a bound ligand, the amount of complementary contact surface between a binding moiety and a ligand, the deformation energy of binding of a given ligand to a binding moiety, and some estimate of hydrogen bonding strength, van der Waals interaction, hydrophobic interaction, and/or electrostatic interaction energies between ligand and binding moiety. Computer modeling can also provide comparisons between the features of a model system and a candidate compound. For example, a computer modeling experiment can compare a pharmacophore model of the invention with a candidate compound to assess the fit of the candidate compound with the model. These modelling, which include computer systems may be used to analyze atomic coordinate data. The minimum hardware means of the computer-based systems of the present invention comprises a central processing unit (CPU) interconnected with appropriate input, outputs and data storage. A monitor may also be used in these systems to visualize structure data. The data storage means be RAM or means for accessing computer readable media.

The computer may produce a three-dimensional representation of a molecule or molecular complex defined by structure coordinates of all of PvcFRS amino acids, or a three-dimensional representation of a homologue of the molecule or molecular complex, wherein the homologue comprises a binding site that has a root mean square deviation from the backbone atoms of the amino acids of up to 6 Å (e.g., up to 5 Å, up to 4 Å, up to 3 Å, up to 2 Å, up to 1.5 Å).

Suitable computers or computer systems are disclosed in U.S. Pat. Nos. 5,978,740 and 6,183,121 (each incorporated by reference by reference in their entirety). For example, a computer system may include a computer comprising a central processing unit (CPU), a working memory (e.g., RAM (random-access memory), core memory), a mass storage memory (e.g., disk drives or CD-ROM drives), one or more display terminals (e.g., cathode-ray tube (CRT), liquid crystal display (LCD), plasma display), one or more keyboards, one or more input lines, and one or more output lines, all of which may be interconnected by a system bus. The display terminal may be used to display a graphical representation of a binding pocket of this invention using a program such as QUANTA or PyMOL. Output hardware might also include a printer, or a disk drive to store system output.

Machine-readable data of this invention may be inputted to the CPU via the use of interconnected systems such as through the internet, modem or modems connected by a data line, electromagnetic radiation, or combinations thereof. Alternatively, or additionally, the input hardware may include CD-ROM drives, disk drives or flash memory. In operation, one or more CPUs may coordinate the use of the various input and output devices, coordinate data accesses from the mass storage and accesses to and from working memory, and/or determine the sequence of data processing steps.

The machine-readable data may be stored on a magnetic storage medium. A magnetic data storage medium can be encoded with a machine-readable data that can be carried out by a system such as the computer system described above. The medium can be a floppy diskette or hard disk or those magnetic storage mediums having a substrate and a coating, on one or both sides of the substrate, containing magnetic domains whose polarity or orientation can be altered magnetically. The magnetic domains of the medium may be polarized or oriented so as to encode machine readable data such as that described herein, for execution by a system such as the computer system described herein. An optically-readable data storage medium also can be encoded with machine-readable data, or a set of instructions, which can be carried out by a computer system. The medium may be a compact disk read only memory (CD-ROM) or a rewritable medium such as a magneto-optical disk which is optically readable and magneto-optically writable. In the case of CD-ROM, as is well known, a disk coating is reflective and is impressed with a plurality of pits to encode the machine-readable data. The arrangement of pits is read by reflecting laser light off the surface of the coating. A protective coating, which preferably is substantially transparent, is provided on top of the reflective coating. In the case of a magneto-optical disk, as is well known, a data-recording coating has no pits, but has a plurality of magnetic domains whose polarity or orientation can be changed magnetically when heated above a certain temperature, as by a laser. The orientation of the domains can be read by measuring the polarization of laser light reflected from the coating. The arrangement of the domains encodes the data as described above.

Structure data, when used in conjunction with a computer programmed with software to translate those coordinates into the 3-dimensional structure of a molecule or molecular complex comprising a binding pocket may be used for a variety of purposes, such as drug discovery.

For example, the structure encoded by the data may be computationally evaluated for its ability to associate with chemical entities. Chemical entities that associate with a binding site of an apicomplexan FRS polypeptide (e.g., apicomplexan cFRS, PcFRS, CcFRS, TcFRS, PvcFRS, PfcFRS, PmcFRS, PocFRS, CpcFRS, TgcFRS) are expected to reduce parasitic biological activity. Such compounds are potential drug candidates. In some embodiments, the compounds identified may bind to one binding site in the polypeptide. In some embodiments, the compounds identified may bind to two binding sites in the polypeptide (e.g., dual inhibitors). In some embodiments, the compounds identified may bind to three binding sites in the polypeptide (e.g., tri-inhibitors). For example, the compounds screened may comprise one or more compounds having the structure of formula (IV):

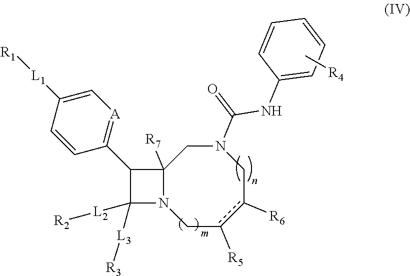

wherein the dashed bond (———) may be a single or double bond;
m is 0 (i.e., it is a bond) or 1;
n is 0, 1 or 2;
A is CH or N;
L₁ is absent, or —C≡C—;
L₂ and L₃ are independently absent, alkylene (e.g., C₁-C₄ alkylene, methylene), or heteroalkylene (e.g., C₁-C₄ heteroalkylene), wherein any of the foregoing groups optionally comprises one or more (e.g., one, two, three) points of substitution;
R₁ is hydrogen, alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, C₁-C₈ heteroalkyl, C₁-C₅ heteroalkyl, C₃-C₁₂ heterocycloalkyl), halogen (e.g., fluoro, chloro), aryl (e.g., C₆-C₁₂ aryl, phenyl), or heteroaryl (e.g., C₅-C₁₂ heteroaryl, pyridinyl), and R₁ has one or more (e.g., two, three, four, five) optional points of substitution;
R₂ and R₃ are independently hydrogen, —OH, —OR, —S(O)₂R, —N(R)S(O)₂R, —C(O)R, —N(R)C(O)R, —N(R)₂, or heterocyclyl, and R₂ and/or R₃ has one or more (e.g., two, three, four, five) optional points of substitution;
R₄ is hydrogen, perfluoroalkyl, aryl (e.g., C₆-C₁₂ aryl, phenyl), arylalkyl (e.g., C₇-C₁₄ alkylaryl, benzyl), alkyl (e.g., C₁-C₁₂ alkyl, C₁-C₈ alkyl, C₁-C₅ alkyl, C₃-C₁₂ cycloalkyl), heteroalkyl (e.g., C₁-C₁₂ heteroalkyl, alkoxy such as C₁-C₁₂ alkoxy or C₃-C₈ cycloalkoxy), or heteroaryl (e.g., C₅-C₁₂ heteroaryl, pyridinyl), and R₄ has one or more (e.g., two, three, four, five) optional points of substitution (e.g., with alkoxy, fluoroalkoxy);
R₅ and R₆ are independently selected from hydrogen and —OH;
R₇ is hydrogen, —CH₂OH, or —CH₂OR;
R is independently selected at each occurrence from hydrogen and alkyl (e.g., C₁-C₁₂ alkyl, C₁-C₈ alkyl, C₁-C₅ alkyl, C₃-C₁₂ cycloalkyl), wherein each R has one or more (e.g., two, three, four, five) optional points of substitution (e.g., with —OH, with C(O)OH, —CN, —NH₂, —N(R^{A})₂); and
R^{A} is independently selected at each occurrence from hydrogen and lower alkyl (e.g., C₁-C₄ alkyl, methyl, ethyl, propyl, isopropyl); or
pharmaceutically acceptable salts thereof; or
prodrugs of any of the foregoing.

Thus, according to another embodiment, the invention relates to a method for evaluating the potential of a chemical entity to associate with a) a molecule or molecular complex comprising a binding site defined by structure coordinates of an apicomplexan enzyme, as described herein, or b) a homologue of said molecule or molecular complex, wherein the complex comprises binding pocket having a root mean square deviation from the backbone atoms of the amino acids of the binding site of 6 Å or less (e.g., 5 Å or less, 4 Å or less, 3 Å or less, 2 Å or less, 1.5 Å or less, from 0.1 Å to 6 Å).

The design of compounds that bind to an apicomplexan FRS binding pocket sequence (e.g., those identified for PvcFRS), that reduce parasitic biological activity may involve consideration of several factors. For example, the compound may physically and/or structurally associate with at least a fragment of an apicomplexan cFRS polypeptide. Non-covalent molecular interactions important in this association include hydrogen bonding, van der Waals interactions, hydrophobic interactions and electrostatic interactions. Desirably, the compound assumes a conformation that allows it to associate with the binding site(s) directly. Although certain portions of the compound may not directly participate in these associations, those portions of the entity may still influence the overall conformation of the molecule. This, in turn, may have a significant impact on the compound's potency. Such conformational requirements include the overall three-dimensional structure and orientation of the chemical compound in relation to all or a portion of the binding site, or the spacing between functional groups comprising several chemical compounds that directly interact with the binding site or a homologue thereof.

The potential inhibitory or binding effect of a chemical compound on an apicomplexan FRS enzyme binding site may be analyzed prior to its actual synthesis and testing by the use of the computer modeling techniques disclosed herein. If the theoretical structure of the given compound suggests insufficient interaction and association between it and the target binding site, testing of the compound may be obviated. However, if computer modeling indicates a strong interaction, the molecule may be synthesized or obtained and tested for its ability to bind the apicomplexan enzyme binding pocket sequence. In some embodiments, the compounds having a strong interaction may be tested by assaying for example, EC50, IC50, or K_{D}, values of the compound identified as an inhibitor candidate such as by performing any of the assays described in Examples 2-4. In various implementations, candidate compounds may be computationally evaluated by means of a series of steps in which chemical entities or fragments are screened and selected for their ability to associate with the PvcFRS binding pocket.

Screening may begin by visual inspection of, for example, an apicomplexan FRS enzyme binding site on the computer screen based on the PvcFRS polypeptide structure coordinates described herein, or other coordinates which define a similar shape generated from the machine-readable storage medium. Selected fragments or chemical compounds may then be positioned in a variety of orientations, or a simulation of the docking within that binding site may occur. Such docking may be accomplished using software such as Quanta and DOCK, followed by energy minimization and molecular dynamics with standard molecular mechanics force fields, such as QUANTA, PyMOL, CHARMM and AMBER. Specific computer software is available in the art to evaluate compound deformation energy and electrostatic interactions. These programs may be implemented, for instance, using a commercially-available graphics workstation.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of the target binding site or analysis of relevant interaction parameters simulated between the compound and the polypeptides input.

Instead of proceeding to build an inhibitor of a binding pocket in a step-wise fashion one fragment or chemical entity at a time as described above, inhibitory or other binding compounds may be designed as a whole or “de novo” using either an empty binding site or optionally including some portion(s) of a known inhibitor(s). There are many de novo ligand design methods known in the art, some of which are commercially available (e.g., LeapFrog, available from Tripos Associates, St. Louis, Mo.). Other molecular modeling techniques may also be employed in accordance with this invention (e.g., T. L. Nero et al. Biochem Soc Trans 46 (2018): 1367-1379, N. C. Cohen et al., J Med. Chem. 33 (1990): 883-894, M. A. Navia et al. Current Opinions in Structural Biology 2 (1992): 202-210; L. M. Balbes et al., Reviews in Computational Chemistry 5, K. B. Lipkowitz and D. B. Boyd, Eds., VCH, New York (1994): 337-380; W. C. Guida, Curr. Opin. Struct. Biology 4 (1994): 777-781, each of which is incorporated by reference in their entirety. Once a compound has been designed or selected, the efficiency with which that entity may bind to a binding site may be tested and optimized by computational evaluation.

Another aspect involves the in silico screening of virtual libraries of compounds. Many thousands of compounds can be rapidly screened, and the best virtual compounds can be selected for further screening (e.g., by synthesis or obtaining from commercial sources and in vitro or in vivo testing). Small molecule databases can be screened for chemical entities or compounds that can bind, in whole or in part, to an apicomplexan FRS binding site. In such screening, the quality of fit of such entities to the binding site may be judged either by shape complementarity or by estimated interaction energy.

In some embodiments, the present disclosure relates to a computer for producing a three-dimensional representation of:
a) a molecule or molecular complex, wherein said molecule or molecular complex comprises a binding pocket of an apicomplexan FRS polypeptide (e.g., apicomplexan cFRS, PcFRS, CcFRS, TcFRS, PvcFRS, PfcFRS, PmcFRS, PocFRS, CpcFRS, TgcFRS) defined by structure coordinates of amino acid residues; or
b) a three-dimensional representation of a homologue of said molecule or molecular complex, wherein said homologue comprises a binding site that has a root mean square deviation from the backbone atoms of amino acids of the FRS polypeptide of 6 Å or less (e.g., 5 Å or less, 4 Å or less, 3 Å or less, 2 Å or less, 1.5 Å or less, from 0.1 Å to 6 Å),
   wherein the computer comprises:
(i) a machine-readable data storage medium comprising a data storage material encoded with machine-readable data, wherein said data comprises the structure coordinates of structure coordinates of amino acid residues in the binding pocket sequence of an apicomplexan FRS polypeptide (e.g., apicomplexan cFRS, PcFRS, CcFRS, TcFRS, PvcFRS, PfcFRS, PmcFRS, PocFRS, CpcFRS, TgcFRS) or fragment thereof;
(ii) a working memory for storing instructions for processing said machine-readable data;
(iii) a central-processing unit coupled to said working memory and to said machine-readable data storage medium for processing said machine readable data into said three-dimensional representation; and
(iv) optionally a display coupled to said central-processing unit for displaying said three-dimensional representation.

The practice of the present invention employs, unless otherwise indicated, conventional techniques of chemistry, molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991), each of which are hereby incorporated by reference in their entirety. These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Synthesis of Compound 1

### General Considerations

Oxygen and/or moisture sensitive reactions were carried out in oven or flame-dried glassware under nitrogen atmosphere. All reagents and solvents were purchased and used as received from commercial vendors or synthesized according to cited procedures. Yields refer to chromatographically and spectroscopically pure compounds, unless otherwise stated. Flash chromatography was performed using 20-40 μm silica gel (60 Å mesh) on a Teledyne Isco Combiflash Rf. Analytical thin layer chromatography (TLC) was performed on 0.2 mm or 0.25 mm silica gel 60-F plates and visualized by UV light (254 nm). NMR spectra were recorded on Bruker 300 (¹H, 300 MHz; ¹³C, 75 MHz) or 400 (¹H, 400 MHz; ¹³C, 100 MHz) or Varian 400MR (¹H, 400 MHz; ¹³C, 100 MHz) spectrometers at 300 K unless otherwise noted. Chemical shifts are reported in parts per million (ppm) relative to the appropriate solvent. Data for ¹H NMR are reported as follows: chemical shift, multiplicity (br=broad, s=singlet, bs=broad singlet, d=doublet, t=triplet, m=multiplet), coupling constants, and integration. Tandem liquid chromatography/mass spectrometry (LCMS) was performed on a Waters 2795 separations module and 3100 mass detector, or alternatively on a Shimadzu LC-20AD separations module or Agilent 1200 series, with data acquired either directly on reaction mixtures or on purified samples.

The reaction scheme for the synthesis of Compound 1 is illustrated below.

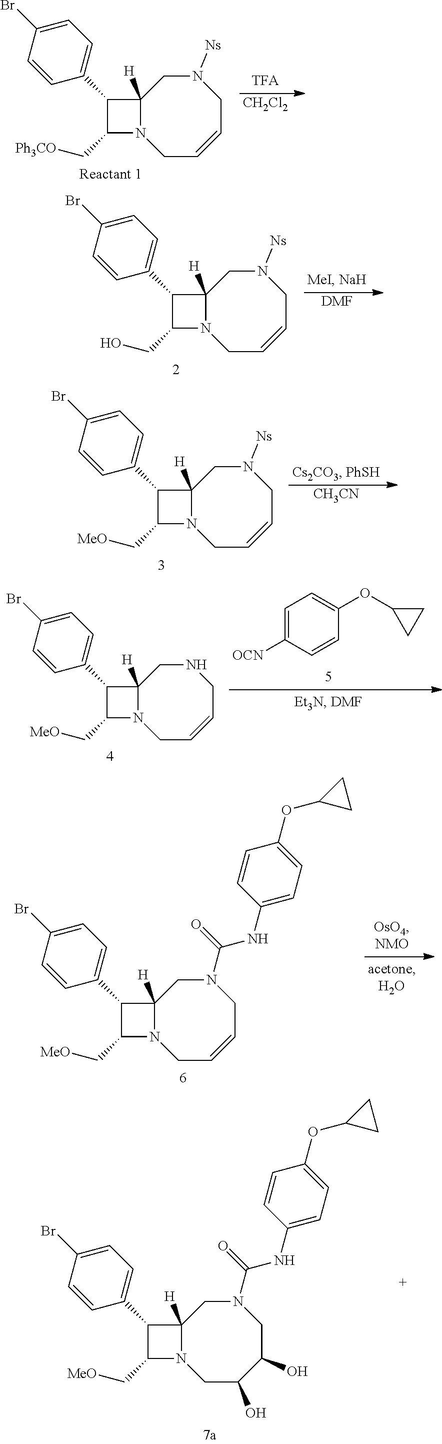

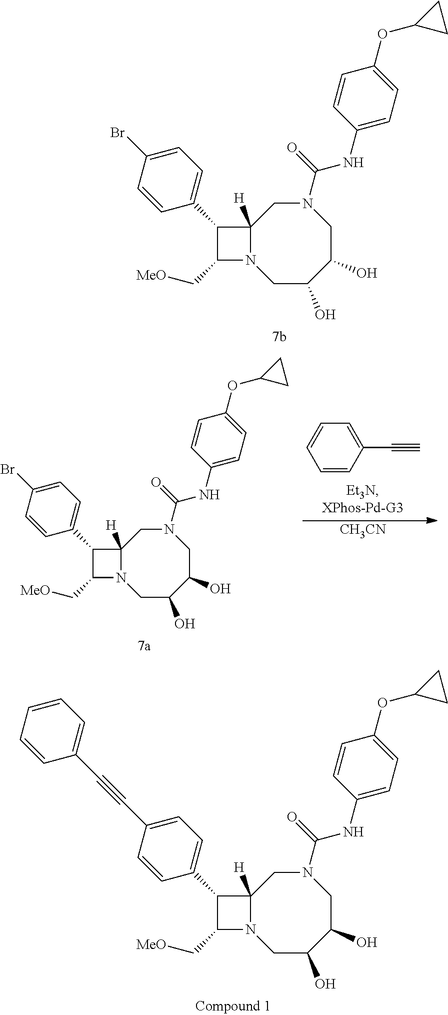

### ((8R,9R,10S,Z)-9-(4-bromophenyl)-6-((4-nitrophenyl)sulfonyl)-1,6-diazabicyclo[6.2.0]dec-3-en-10-yl)methanol (2)

(8R,9R,10S,Z)-9-(4-bromophenyl)-6-((4-nitrophenyl)sulfonyl)-10-((trityloxy)methyl)-1,6-diazabicyclo[6.2.0]dec-3-ene (8.00 g, 10.7 mmol, 1.00 equiv) (Reactant 1, prepared according to the method of Lowe, J. T. et al., J Org. Chem 77 (2012): 7187-7211, which is hereby incorporated by reference in its entirety) was dissolved in CH₂Cl₂ (100 mL) and TFA was added (15.8 mL, 213 mmol, 20.0 equiv). The mixture was stirred at 15° C. for 6 hours. After completion, the reaction was quenched by addition of sat. aq. NaHCO₃ until pH=8, and then extracted with CH₂Cl₂ (3×30 mL). The combined organic layers were washed with brine (50 mL), dried over Na₂SO₄, filtered and concentrated in vacuo. The resulting mixture was partially purified by column chromatography (SiO₂, petroleum ether/ethyl acetate=20:1 to 0:1) to afford a crude brown solid (3.80 g). A portion of this material was engaged in the next step without further purification.

### (8R,9R,10S,Z)-9-(4-bromophenyl)-10-(methoxymethyl)-6-((4-nitrophenyl)sulfonyl)-1,6-diazabicyclo[6.2.0]dec-3-ene (3)

Alcohol 2 (crude, 1.00 g aliquot, 1.97 mmol, 1.00 equiv) was dissolved in DMF (10 mL). Sodium hydride (60% dispersion in mineral oil, 236 mg, 5.90 mmol, 3.00 equiv) was added dropwise at 0° C. under N₂. The reaction mixture was stirred at 0° C. for 1 hour. Then iodomethane (1.12 g, 7.87 mmol, 489 μL, 4.00 equiv) was added and the mixture was heated to 25° C. and stirred for 11 hours. After completion, the reaction was cooled to 0° C., quenched by addition of H₂O (10 mL), and extracted with EtOAc (3×30 mL). The combined organic layers were dried over Na₂SO₄, filtered and concentrated in vacuo. The resulting mixture was partially purified by column chromatography (SiO₂, petroleum ether/ethyl acetate=10:1 to 1:1) to afford a crude yellow oil (380 mg) engaged in the next step without further purification.

### (8R,9R,10S,Z)-9-(4-bromophenyl)-10-(methoxymethyl)-1,6-diazabicyclo[6.2.0]dec-3-ene (4)

Sulfonamide (Ns) 3 (crude, 380 mg, 0.727 mmol, 1.00 equiv) was dissolved in CH₃CN (10 mL). Cs₂CO₃ (474 mg, 1.45 mmol, 2.00 equiv) and benzenethiol (120 mg, 1.09 mmol, 111 μL, 1.50 equiv) were then added in one portion and the mixture was heated at 40° C. After 2 hours, the reaction was quenched by addition of H₂O (10 mL) and then extracted with CH₂Cl₂ (3×10 mL). The combined organic layers were dried over Na₂SO₄, filtered and concentrated in vacuo. The resulting mixture was partially purified by preparative TLC (SiO₂, petroleum ether/ethyl acetate=0:1) to afford a crude yellow oil (160 mg). A portion of this material was engaged in the next step without further purification. 1-cyclopropoxy-4-isocyanatobenzene (5)

To a solution of bis(trichloromethyl) carbonate (19.7 mg, 67.0 μmol, 0.500 equiv) in toluene (0.260 mL) at 20° C., under N₂, was added dropwise a solution of 4-(cyclopropoxy)aniline (19.8 mg, 0.133 mmol, 1.00 equiv, prepared according to the method of disclosed in Int'l Pub No WO 2013149996, which is hereby incorporated by reference in its entirety) in dioxane (47.0 μL). The resulting mixture was warmed to 110° C. and stirred for 1 hour. During this period, the initial suspension turned into a clear mixture, which was concentrated in vacuo and used in the next step without further purification.

### (8R,9R,10S,Z)-9-(4-bromophenyl)-N-(4-cyclopropoxyphenyl)-10-(methoxymethyl)-1,6-diazabicyclo[6.2.0]dec-3-ene-6-carboxamide (6)

Amine 4 (crude, 30.0 mg aliquot, 89.0 μmol, 1.00 equiv) was dissolved in CH₂Cl₂ (1.77 mL). Et₃N (24.6 μL, 0.177 mmol, 2.00 equiv) and isocyanate 5 (23.2 mg, 0.133 mmol, 1.50 equiv, prepared as described above) were added at 0° C. under N₂. The mixture was stirred at 20° C. for 30 min and concentrated in vacuo. The residue was purified by column chromatography (SiO₂, ethyl acetate/hexane=0:1 to 7:3) to afford the desired compound (24.2 mg, calculated yield: 8.9% from 1).

LC-MS m/z calculated for C₂₆H₃₀BrN₃O₃Na[M+Na]⁺534.15; Found 534.17.

1H NMR (400 MHz, chloroform-d) δ 7.45 (d, J=7.8 Hz, 2H), 7.35 (d, J=8.1 Hz, 2H), 7.20 (d, J=8.4 Hz, 2H), 6.94 (d, J=8.4 Hz, 2H), 6.10 (s, 1H), 5.88-5.78 (m, 1H), 5.76-5.67 (m, 1H), 4.20 (d, J=16.8 Hz, 1H), 3.98 (dd, J=16.4, 7.3 Hz, 1H), 3.74-3.39 (m, 7H), 3.22 (d, J=5.5 Hz, 1H), 3.16 (s, 4H), 2.85 (t, J=12.4 Hz, 1H), 0.73 (d, J=4.5 Hz, 4H).

### (3S,4R,8R,9R,10S)-9-(4-bromophenyl)-N-(4-cyclopropoxyphenyl)-3,4-dihydroxy-10-(methoxymethyl)-1,6-diazabicyclo[6.2.0]decane-6-carboxamide (7a)

Olefin 6 (24.2 mg, 47.0 μmol, 1.00 equiv) was dissolved in acetone (0.393 mL) and water (76.0 μL). 4-methylmorpholine N-oxide (19.0 μL, 94.0 μmol, 2.00 equiv) and osmium(VIII) tetroxide solution (4 wt. % in H₂O, 2.90 μL, 0.470 μmol, 0.0100 equiv) were added at 25° C., and the mixture was stirred at this temperature for 16 h. Next, the mixture was dried over Na₂SO₄, filtered and concentrated in vacuo. The resulting mixture was purified by flash column chromatography (SiO₂, ethyl acetate/hexane=0:1 to 1:0) to afford the desired compound 7a (9.00 mg, yield: 35%) and its diastereomer 7b (see below, 13.1 mg, yield: 45%).

LC-MS m/z calculated for C₂₆H₃₂BrN₃O₅Na[M+Na]⁺568.15; Found 568.24.

1H NMR (400 MHz, chloroform-d) δ 7.77 (bs, 1H), 7.43 (d, J=8.0 Hz, 2H), 7.34 (d, J=8.2 Hz, 2H), 7.18 (d, J=8.4 Hz, 2H), 6.95 (d, J=8.5 Hz, 2H), 4.29 (dd, J=16.1, 5.5 Hz, 1H), 4.07 (d, J=5.0 Hz, 1H), 3.91-3.59 (m, 5H), 3.56-3.37 (m, 3H), 3.29-3.17 (m, 2H), 3.15 (s, 3H), 2.95-2.84 (m, 1H), 2.83-2.74 (m, 1H), 2.68 (t, J=12.5 Hz, 1H), 2.29 (bs, 1H), 0.73 (d, J=4.4 Hz, 4H).

### (3R,4S,8R,9R,10S)-9-(4-bromophenyl)-N-(4-cyclopropoxyphenyl)-3,4-dihydroxy-10-(methoxymethyl)-1,6-diazabicyclo[6.2.0]decane-6-carboxamide (7b)

LC-MS m/z calculated for C₂₆H₃₂BrN₃O₅Na[M+Na]⁺568.15; Found 568.24.

1H NMR (400 MHz, chloroform-d) δ 7.49 (d, J=8.0 Hz, 2H), 7.35-7.24 (m, 4H), 6.98 (d, J=8.5 Hz, 2H), 6.60 (bs, 1H), 3.87 (d, J=7.0 Hz, 1H), 3.81-3.58 (m, 6H), 3.57-3.39 (m, 3H), 3.35-3.23 (m, 2H), 3.20 (s, 3H), 2.88 (bs, 1H), 2.82 (d, J=14.1 Hz, 1H), 1.46 (d, J=23.2 Hz, 1H), 0.76 (d, J=4.5 Hz, 4H), 1 exchangeable proton not observed.

### (3S,4R,8R,9R,10S)—N-(4-cyclopropoxyphenyl)-3,4-dihydroxy-10-(methoxymethyl)-9-(4-(phenylethynyl)phenyl)-1,6-diazabicyclo[6.2.0]decane-6-carboxamide (Compound 1)

A sealed vial containing aryl bromide 7a (22.1 mg, 40.0 μmol, 1.00 equiv) was evacuated and backfilled with N₂ (×3) then were added CH₃CN (0.400 mL, previously sparged with argon for 40 min), Et₃N (22.3 μL, 0.161 mmol, 4.00 equiv) and phenylacetylene (22.1 μL, 0.202 mmol, 5.00 equiv), followed by (2-Dicyclohexylphosphino-2′,4′,6′-triisopropyl-1,1′-biphenyl)[2-(2′-amino-1,1′-biphenyl)]palladium(II) methanesulfonate (XPhos-Pd-G3, 3.40 mg, 4.00 μmol, 0.100 equiv). The vial was sealed and heated to 70° C. After 90 min, the reaction was allowed to cool at room temperature, sat. aq. NaHCO₃ was added, and the mixture was extracted with CH₂Cl₂ (3×0.40 mL). The combined organic layers were dried over Na₂SO₄, filtered and concentrated in vacuo. The residue was purified by flash column chromatography reverse phase (C18, CH₃CN/water=0:1 to 1:1) to afford Compound 1 (16.0 mg, yield: 70%).

LC-MS m/z calculated for C₃₄H₃₈N₃O₅ [M+H]⁺ 568.26; Found 568.86.

1H NMR (400 MHz, chloroform-d) δ 7.73 (bs, 1H), 7.58-7.42 (m, 6H), 7.40-7.31 (m, 3H), 7.19 (d, J=8.5 Hz, 2H), 6.95 (d, J=8.8 Hz, 2H), 4.30 (dd, J=16.0, 5.5 Hz, 1H), 4.12-4.05 (m, 1H), 3.94-3.74 (m, 3H), 3.72-3.61 (m, 2H), 3.56 (t, J=7.6 Hz, 1H), 3.48 (dd, J=10.1, 5.6 Hz, 2H), 3.32-3.20 (m, 2H), 3.15 (s, 3H), 2.92 (dd, J=13.6, 9.2 Hz, 1H), 2.77 (dd, J=26.0, 13.1 Hz, 2H), 2.33 (s, 1H), 0.73 (d, J=4.4 Hz, 4H).

### Example 2: Pharmacokinetic Profile of Inhibition

Compound 1 ((3S,4R,8R,9R,10S)—N-(4-cyclopropoxyphenyl)-3,4-dihydroxy-10-(methoxymethyl)-9-(4-(phenylethynyl)phenyl)-1,6-diazabicyclo[6.2.0]decane-6-carboxamide) having the structure:

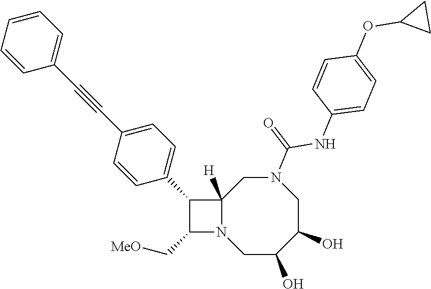 [SMILES: COC[C@@H]1[C@H](c2ccc(C#Cc3ccccc3)cc2)[C@@H]2CN(C(=O)Nc3ccc(OC4CC4)cc3)C[C@@H](O)[C@@H](O)CN12]

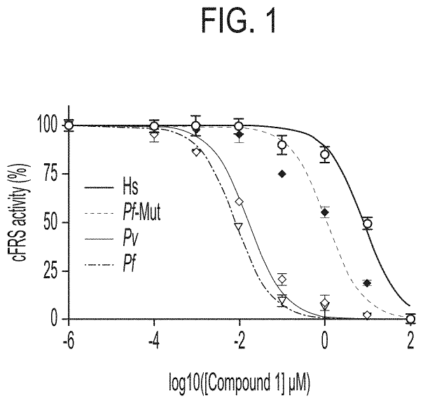

was shown to have high in vitro potency in the growth inhibition assay (Pf Dd2 EC50<1 nM), potent abrogation of parasite FRS enzyme activity, and very high selectivity index over the human orthologue. EC₅₀ assays were run as described in Int'l Pub Nos. WO 2015070204, WO 2018175385, and Kato, N, et al. Nature 538 (2016): 344-349, which are hereby incorporated by reference in its entirety. The inhibition of the aminoacylation activity of Pf (Plasmodium falciparum), Pv (Plasmodium vivax), Hs (Human) and Pf-Mut (L₅₅₀V) cFRS enzymes by Compound 1 are illustrated in FIG. 1. These assays were performed at concentrations ranging from 0.001 nM to 100 μM and IC50 values were calculated by non-linear regression.

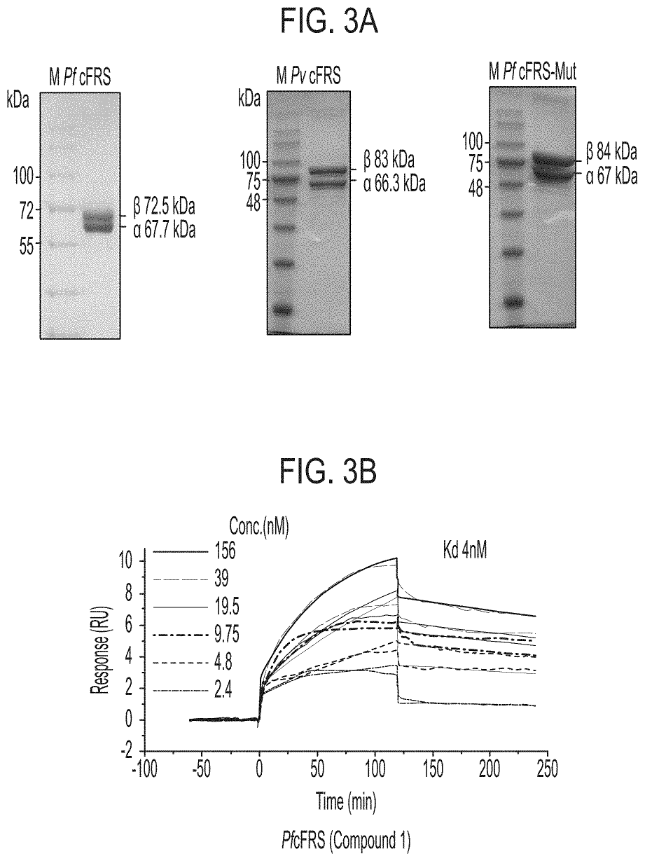

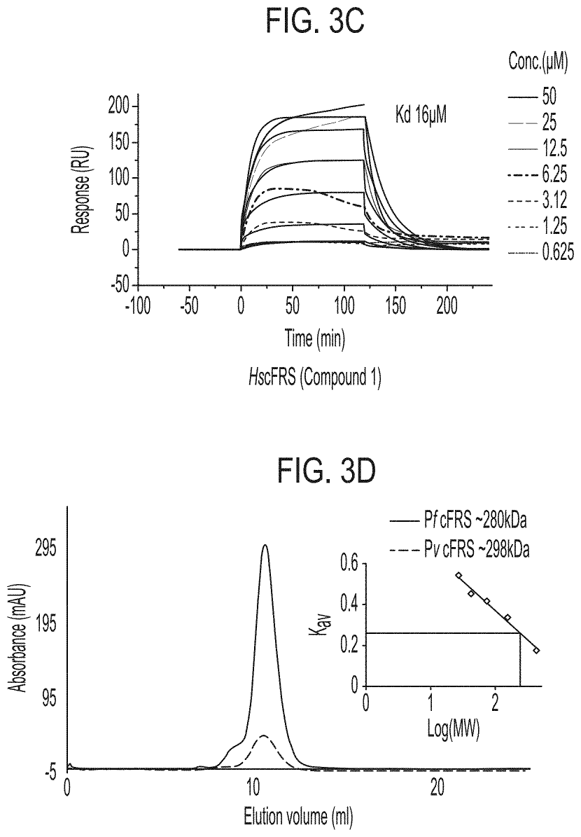

Surface Plasmon Resonance experiments to evaluate the binding of Compound 1 to proteins were carried out on a Biacore T200 instrument (GE Healthcare) at 25° C. The binding experiments were performed in buffer 10 mM phosphate buffered saline (PBS), pH 7.4, containing 5% dimethyl sulfoxide (DMSO). The flow system was primed with the running buffer before the initiation of the experiment. Both PfcFRS and HscFRS were immobilized to Sensor Chip CM5 by standard amine coupling chemistry using N-hydroxysuccinimide (NHS) and ethyl(dimethylaminopropyl) carbodiimide (EDC), to an immobilization level of approximately 1500 RU. The binding experiments were carried out in a single cycle kinetics mode. Compound 1 was serially diluted in running buffer, and injected at a flow rate of 60 μl min⁻¹ across both surface for 60 s and dissociation were set up for 120 s. The analysis was done using Biaevaluation (GE Healthcare) and GraphPad Prism 7 software (GraphPad Software Inc, USA). The reference flow cell was left unmodified and the data from the reference flow cell were subtracted for all runs. The equilibrium dissociation constants (K_{d}) were determined by plotting the measured response (R_{eq}) as a function of the analyte concentration. The data was further fitted to a 1:1 binding model of nonlinear regression (specific binding) using Graph Pad Prism 6.0. The experiments were performed in duplicates. Measured sensograms illustrating the binding of various concentrations of Compound 1 to the protein are shown in FIG. 3B (PfcFRS) and 3C (HscFRS) including both the measured data and the 1:1 binding model fit.

Table 1 illustrates the measured half maximal effective Pf Dd2 growth inhibition concentrations (EC₅₀), half maximal inhibitory response (IC₅₀), and equilibrium dissociation constants (K_{d}) for Compound 1 on Pf (Plasmodium falciparum), Pv (Plasmodium vivax), Hs (Human) and Pf-Mut (L550V) cFRS.

TABLE 1

| | | | Measurement |
|---|---|---|---|
| | Assay | | (nM) |
| PfDd2 growth inhibition EC₅₀ (nM) | | | <1 |
| | cFRS IC₅₀ (nM) | Pf | 12 |
| | | Pv | 25 |
| | | Pf mutant (L550V) | 1.0 × 10³ |
| | | Hs | 12 × 10³ |
| | cFRS K_{d} (nM) | Pf | 4 |
| | | Hs | 16 × 10³ |

The unique structural properties of the presently disclosed compounds result in dramatic increases to efficacy. For example, Compound 1 has three fold better efficacy as measured by the EC₅₀ Pf Dd2 growth inhibition than (3S,4R,8R,9S,10S)—N-(4-cyclopropoxyphenyl)-10-((dimethylamino)methyl)-3,4-dihydroxy-9-(4-(phenylethynyl)phenyl)-1,6-diazabicyclo[6.2.0]decane-6-carboxamide.

The enzymatic activity of purified heterodimeric PfcFRS using malachite green-based aminoacylation assays with substrates L-Phe and ATP was also measured as described in Cestari, I. et al., J Biomol. Screen. 18 (2013): 490-497 and Kato, N, et al. Nature 538 (2016): 344-349, which are hereby incorporated by reference in its entirety. Briefly, the aminoacylation reaction was observed for 100 μM ATP, 50 μM L-phenylalanine and 100 nM recombinant PheRS enzymes (Pf Pv, Hs, Pf-Mut) in a buffer containing 30 mM HEPES (pH 7.5), 150 mM, NaCl, 30 mM KCl, 50 mM, MgCl₂, 1 mM DTT and 2 U/ml E. coli inorganic pyrophosphatase (NEB) at 37° C. Enzymatic reactions (50 μl total volume) were performed in clear, flat-bottomed, 96-well plates (Costar 96-well standard microplates). The reaction mixture was incubated for 2 h at 37° C. The reaction was stopped by adding 12.5 μl of malachite green solution to the reaction mixture and levels of inorganic phosphate (Pi) were detected after incubation of 5 min at room temperature. Absorbance was the measured at 620 nm using a Spectramax M2 (Molecular Devices). Reactions without FRS enzyme were performed as background controls, values of which were subtracted from the reactions with enzyme. Compound 1 was added to the aminoacylation assay reaction buffer in varying concentrations ranging from 0.1 nM to 10,000 nM.

Using Graph Pad Prism each data set was individually fitted to the Michaelis-Menten equation and the resulting Lineweaver-Burk plots were examined for diagnostic patterns of competitive, mixed, or uncompetitive inhibition. Data sets were then globally fitted to the appropriate model (with equations (1) and (2) used for competitive and mixed inhibition respectively).

$$\begin{matrix}v = \frac{Vmax \cdot [ S ]}{{K}_{m} ( 1 + \frac{[ I ]}{{K}_{i}} ) + [ S ]} & ( 1 ) \\ v = \frac{Vmax \cdot [ S ]}{{K}_{m} ( 1 + \frac{[ I ]}{{K}_{i}} ) + {K}_{m} ( 1 + \frac{[ I ]}{{K}_{i}} ) [ S ]} & ( 2 )\end{matrix}$$

where v is the reaction rate, Kₘ is the Michaelis-Menten constant, Vₘₐₓ is the maximum reaction velocity, Kᵢ is the inhibitor constant, [I] is the concentration of inhibitor (Compound 1) and [S] is the concentration of substrate.

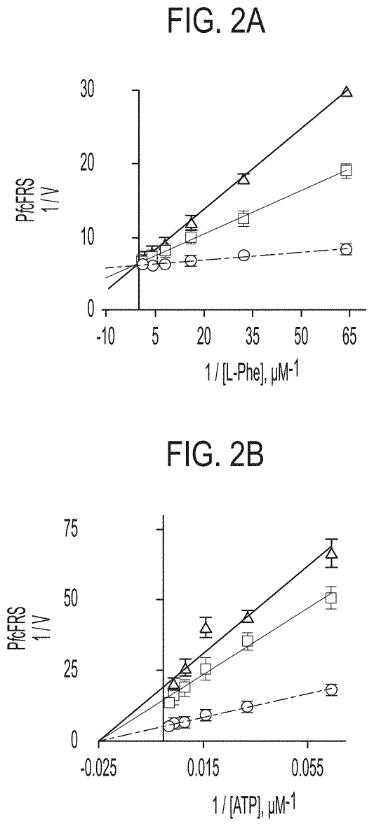

The data is shown for three replicates as the mean±SD in FIGS. 2A and 2B. Compound 1 is a competitive inhibitor of PfcFRS with respect to L-Phe (Kᵢ=6 nM) while a non-competitive inhibitor with respect to ATP (Kᵢ=10 nM). Specifically, enzyme activity was evaluated at a fixed saturating concentration of one substrate (L-Phe or ATP) and varying concentrations the other at different inhibitor concentrations. The experimental data were fit to the Michaelis-Menten equation that produced Lineweaver-Burk plots as described above. These were then assessed for modes of competitive, mixed, or uncompetitive inhibition. The data indicate that Compound 1 displays a mode of competitive inhibition vis-a-vis L-Phe binding (FIG. 2A) as fitting to a global competitive inhibition model resulted in Kᵢ of 6±2 nM for the compound. These results are consistent with previously reported data on in vitro parasite growth inhibition by another bicyclic azetidine which showed higher EC₅₀ values with increasing concentrations of L-Phe as described in Kato, N, et al. Nature 538 (2016): 344-349, which is hereby incorporated by reference in its entirety. The mode of inhibition was then analyzed using ATP as the variable-concentration substrate (FIG. 2B). The data were then fit to the modified high-substrate inhibition Michaelis-Menten equation that led to the corresponding double-reciprocal plot and its inhibition profile. These data were fit to a non-competitive inhibition model where Compound 1 gave Kᵢ value of 10±5 nM.

Without intending to be bound by theory, based on these results, it is likely that Compound 1 preferentially binds to the free enzyme via competition of L-Phe. This mechanism of action is unique when compared to other promising anti-malarial aaRS inhibitors of cladosporin class (requires the presence of L-lysine for its binding) or to halofuginone (requires ATP for its tight binding).

### Example 3: Crystallization of Protein with Inhibitor

Much of the crystallization and analysis of apicomplexan proteins in connection with Compound 1 as described in the present application was performed at the International Centre for Genetic Engineering and Biotechnology by Amit Sharma and his lab as described in M Sharma, N Malhotra, M. Yogavel, K. Harlos, B Melillo, E. Comer, A. Gonse, B. Mitasev, F. G. Fang, S. L. Schreiber, and A Sharma, “Structural basis of malaria parasite phenylalanine tRNA-synthetase inhibition by bicyclic azetedines,” Nature communications 12.1 (2021): 1-10, which is hereby incorporated by reference in its entirety. Protein sequences were aligned using the program Cluster W. All structural superimpositions and preparation of figures was conducted using Chimera as disclosed in Pettersen, E. F., et al., Acta Crystallogr. Sect. D Biol Crystallogr. 72 (2015): 87-88, which is hereby incorporated by reference in its entirety.

Briefly, full-length PfcFRS was purified according as described in Kato, N. et al. Nature 538 (2016): 344-349 (2016), which is hereby incorporated by reference in its entirety, and particularly in relation to FRS purification. Full-length PvcFRS was also purified according to the same protocol. In brief, the gene encoding PvcFRS alpha subunit (PVX_081300), and beta subunit (PVX_090880) was cloned into E. coli plasmid pETM11 and pETM41 respectively. Both plasmids were co-transformed into E. coli strain B834 and were induced overnight for overexpression with 0.5 mM isopropyl-β-D-thiogalactoside (IPTG) at 18 C for 16h. The E. coli cell lysate was first loaded onto a nickel-nitrilotriacetic (Ni-NTA) column (GE Healthcare), and the elution fraction was further purified with Heparin chromatography (GE Healthcare) to single bands for both alpha (66.3 kDa) and beta (83 kDa) subunits as indicated on sodium dodecyl sulfate-polyacrylamide gel electrophoresis (SDS-PAGE) with Coomassie brilliant blue staining. The electrophoresis of the final purified PfcFRS, PvcFRS, and PfcFRS-MUt proteins are shown in FIG. 3A illustrating the weight of each of the α and β subunits. The purified protein was found as a single peak with the elution volume consistent with a homogeneous PvcFRS Hetero-tetramer on a Superdex 200 analytical gel filtration column (GE Healthcare). The purified PvcFRS was concentrated to 25 mg ml⁻¹ and stored at −80° C. in 25 mM HEPES buffer, pH 7.5, 200 mM NaCl, 5 mM βME.

The purified PfcFRS and PvcFRS proteins were used for crystallization by the hanging-drop vapour-diffusion method at 293 K using commercially available crystallization screens (Index, JCSG-plus, Morpheus, PACT premier, PGA, Crystal Screen, PEG/Ion and ProPlex; Hampton Research and Molecular Dimensions). Initial screening was performed in 96-well plates using a nano drop dispensing Mosquito robot (TTP Labtech). Three different drop ratios were used for the crystallization trials by mixing 75, 100 or 50 nl purified protein solution with 75, 50 or 100 nl reservoir solution, respectively (i.e. 1:1, 2:1 and 1:2 drop ratios). Each of the drops was equilibrated against 100 ml of the corresponding reservoir solution. Before crystallization, PvcFRS was diluted to 12 mg ml⁻¹ with 3 mM Compound 1, 5 mM MgCl₂ and 4 mM βME, and then incubated on ice for 30 min.

The diffraction quality PvcFRS: Compound 1 crystals were obtained in PGA screen F4 [0.1 M sodium cacodylate (pH 6.5), 3% w/v poly-γ-glutamic acid (Na⁺ form, low molecular weight), 3% w/v PEG20000, 0.1 M ammonium sulphate, 0.3 M sodium formate). The crystals were mounted in nylon loops (Hampton Research) or litho loops (Molecular Dimensions) after being soaked for 10-30 s in a cryoprotectant containing the corresponding crystallization mother liquor with 20% (v/v) glycerol. The crystals were subsequently flash-cooled in liquid nitrogen. X-ray diffraction data set were collected on beamline 102 at Diamond Light Source (DLS), United Kingdom at a wavelength of 0.9688 Å. The data were processed by the xia2 auto-processing pipeline using DIALS (Winter, et al., Acta Crystallogr. Sect D Biol. 69 (2013): 1260-1273 and Winter G. et al., Acta Crystallogr. Sect D Struct. Biol. 74 (2018): 85-97, each of which are hereby incorporated by reference in their entirety) for integration. The initial model for PvFRS: Compound 1 was determined by the molecular-replacement (MR) method using Phaser (McCoy, A. J., et al., J Appl. Crystallogr. 40 (2007): 658-674, which is hereby incorporated by reference in its entirety) with HsFRS (Protein Data Bank Entry (PDB) 3L₄G) as the template. The structure was further refined by iterative cycles of refinement with Refmac (Murshudov, G. N. et al., Acta Crystallogr. Sect. D Biol. Crystallogr. 67 (2011): 355-367, which is incorporated by reference in its entirety) and Phenix (Adams, P. D. et al., Acta Crystallogr. Sect. D Biol. Crystallogr. 66 (2010): 213-221, which is incorporated by reference in its entirety) and model building with COOT (Emsley, P. et al. Acta Crystallogr. Sect. D Biol. Crystallogr. 60 (2004): 2126-2132, which is hereby incorporated by reference in its entirety).

The final model was refined to 3.0 Å resolution with Rwork/Rfree of 22.9/28.2%. The stereo-chemical quality of the model was analyzed using MolProbity (Chen, V. B. et al. Acta Crystallogr. Sect. D Biol. Crystallogr. 66 (2010): 12-21, which is hereby incorporated by reference in its entirety) and the model has good geometry quality and 97.9% residues are in favored/allowed regions of the Ramachandran plot. The statistics of data collection and structure refinement are shown in Table 2.

TABLE 2
PvFRS: Compound 1 binary complex

| PDB code | 7BY6 | |
|---|---|---|
| Data collection | | |
| Beamline | I24 | |
| Wavelength (Å) | 0.9688 | |
| Detector type | PILATUS3 6M, S/N 60-0119 | |
| Crystal-to-detector distance (mm) | 580 | |
| Oscillation (°) | 0.1 | |
| Exposure (s) | 0.020 | |
| Beam size (μm) | 50 × 50 | |
| Flux (photons s⁻¹) | 2.30 e⁺⁰ | |
| Transmission (%) | 100 | |
| No. of images | 1800 | |
| Software used for data processing | xia2 | |
| Space group | P 2₁ 2₁ 2 | |
| Cell dimensions | | |
| a, b, c (Å) | 136.53, 74.08, 121.69 | |
| α, β, γ (°) | 90.0, 90.0, 90.0 | |
| Resolution (Å) | 121.69-3.00 | (3.05-3.00)* |
| Rₘₑₐₛ (%) | 0.145 | (1.253) |
| I/σI | 8.4 | (1.7) |
| Completeness (%) | 99.8 | (95.0) |
| Redundancy | 6.3 | (6.3) |
| CC_{1/2} | 0.9 | (0.8) |
| No. of unique reflections | 25457 | (1191) |

| Refinement | |
|---|---|
| Resolution (Å) | 121.69-3.00 |
| No. of reflections/test set | 25353/1295 |
| R_{work}/R_{free} (%) | 22.7/28.2 |
| No. of protein residues | Chain A: 296 |
| | Chain B: 602 |
| No. atoms | |
| Protein | Chain A: 2424 |
| | Chain B: 4682 |
| Ligand (non-H atoms) | 42 |
| Mg²⁺ ion | 1 |
| Average B-factors (Å²) | |
| Protein | 94.7 |
| Ligand | 78.6 |
| Mg²⁺ ion | 79.7 |
| R.m.s deviations | |
| Bond lengths (Å) | 0.003 |
| Bond angles (°) | 0.678 |
| Ramachandran plot | |
| Favoured/Allowed (%) | 92.0/8.0 |

As shown in Table 2, the atomic coordinates and structural factors have been deposited into Protein Data Bank with accession code 7BY6. The PvcFRS: Compound 1 complex was crystallized and crystals that diffracted to 3 Å were obtained.

The structure was solved by molecular replacement using human cytosolic FRS as a template (PDB: 3L₄G). The PvcFRS: Compound 1 crystals belong to orthorhombic space group P2_{I}2_{I}2 with one heterodimer (α1β1) per asymmetric unit. The α2β2 biological heterotetrametric assembly is completed via the crystallographic two-fold axis along c. The PvcFRS α2β2 assembly is consistent with the size exclusion chromatography profile of purified protein, where it elutes at a size of 298 kDa (FIG. 3D).

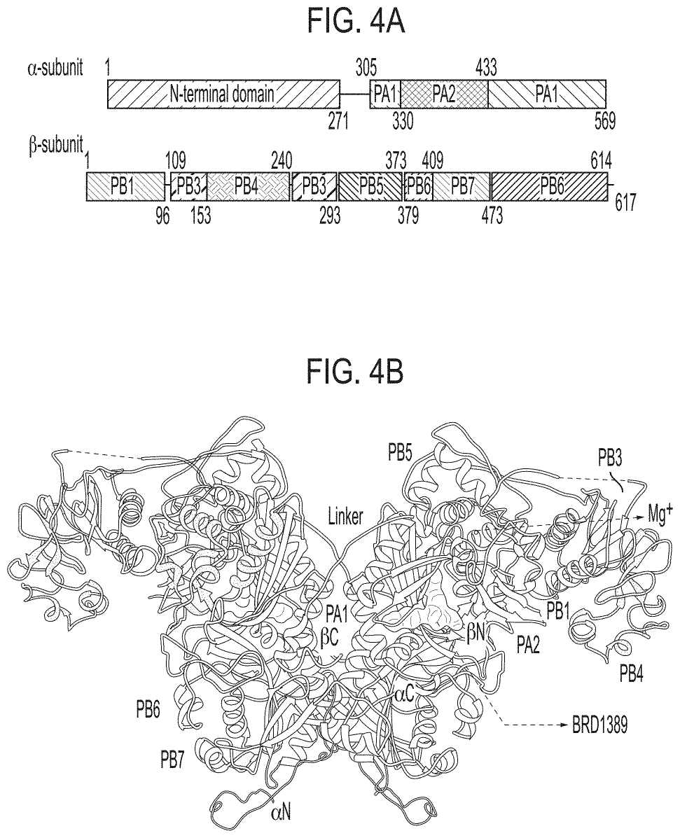

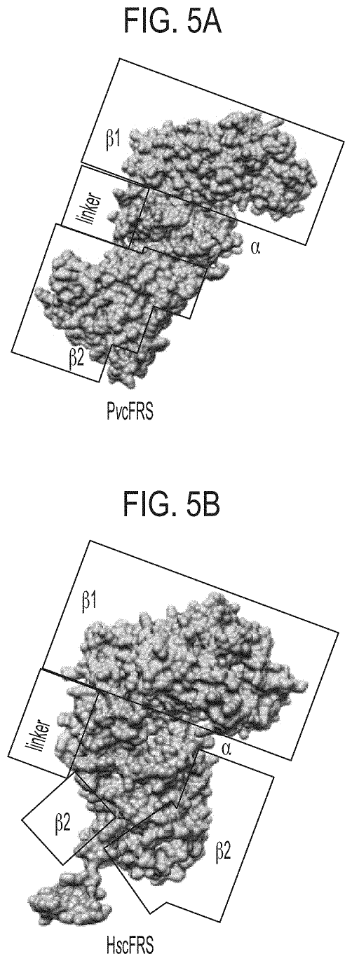

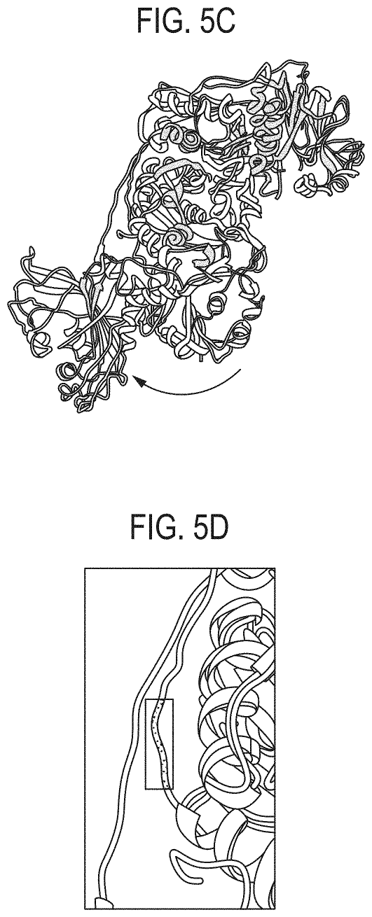

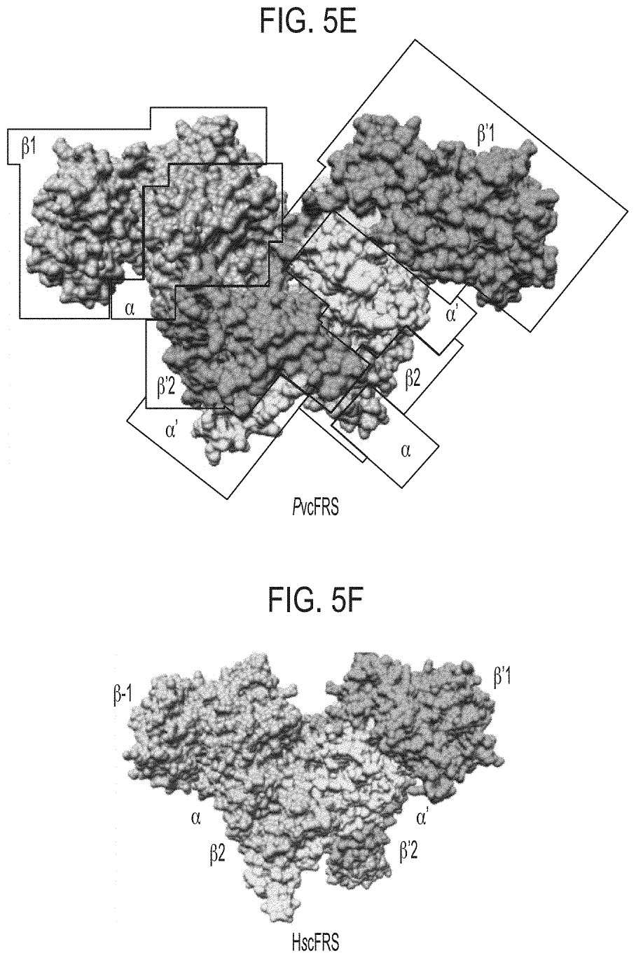

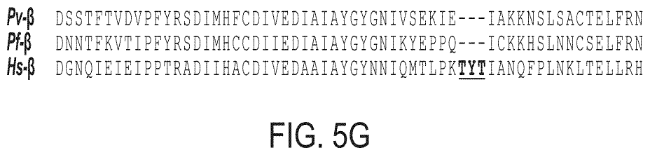

The final PvcFRS: Compound 1 atomic model contained 1126 residues, one Mg²⁺ and one ligand (Compound 1) per α1β1. The N-terminal DNA binding domain (residues 1-270) of the a subunit was found to be disordered and no electron density was observed therein in the calculations. The overall fold and organization of a and R subdomains is very similar to that of the human orthologue (HscFRS, FIGS. 4A and 4B) with the exception that the PvcFRS β subunit lacks an 18-residue-long fragment within its PB1 domain. The association of β1 and β2 subdomains (of the β subunit) with a subunit is significantly different between the parasite and human enzymes (FIGS. 5A-F). In HscFRS, the a subunit is enclosed by β1 and β2 subdomains of the same β subunit, whereas in PvcFRS β1 and β2′ of the β subunit is associated with the a subunit (FIGS. 5E and 5E). This difference arises due to the shorter linker length between 01 and 32 of PvcFRS when compared to the human cFRS (FIGS. 5D and 5G), which has a three residue (384-TYT-386) insertion. Interestingly, this three-residue shorter linker is observed in all human malaria parasites (FIGS. 5D and 5G) and is suggestive of domain swapping in PvcFRS (FIG. 5E) to form a closed, functional hetero-tetrameric (α2β2) assembly.

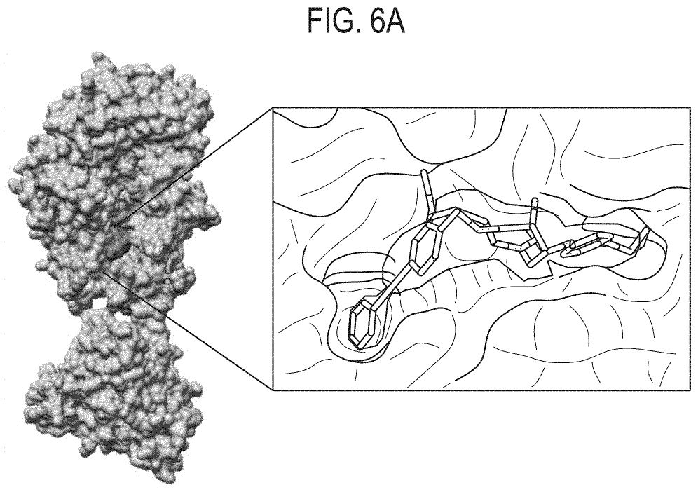

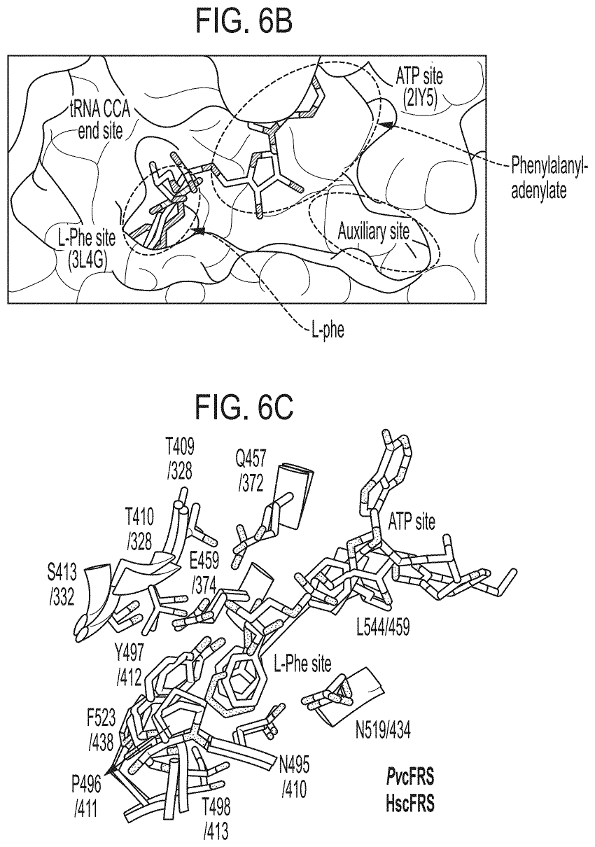

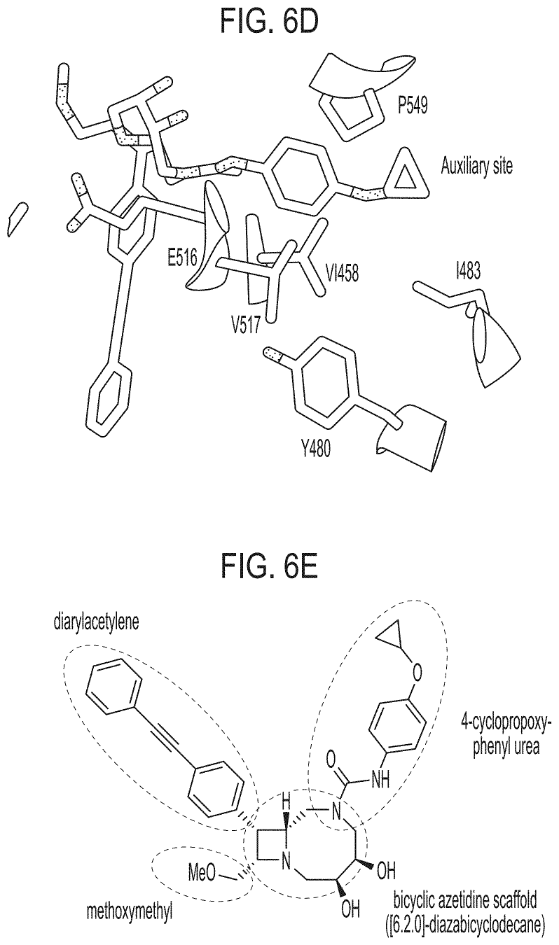

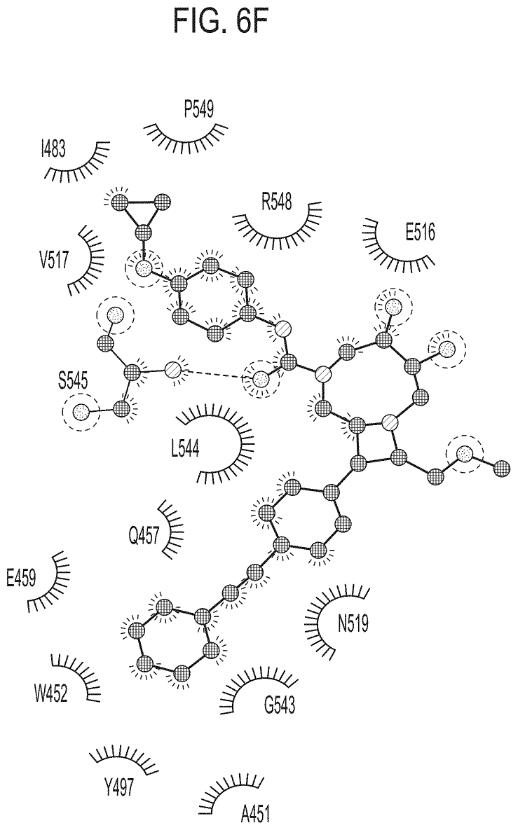

An investigation of difference Fourier electron density (Fₒ-Fₑ) maps of the PvcFRS protein model revealed Compound 1 bound at the enzyme active site (FIG. 6A). The bound ligand was subsequently verified by simulated annealing omit (SA-omit) map (right image in FIG. 6A). Superposition of the PvcFRS α subunit on human cFRS and Thermus thermophilus FRS (TtFRS, PDB: 2IY5) allowed the mapping of L-Phe, ATP and tRNA binding sites on these FRSs (FIG. 6B). Further analysis of the PvcFRS: Compound1 complex revealed that the diarylacetylene moiety of Compound 1 occupies the L-Phe site, its 4-cyclopropoxy phenyl resides in an auxiliary pocket, and the [6.2.0]-diazabicyclodecane group skirts the ATP site in PvcFRS (FIGS. 6C-6E).

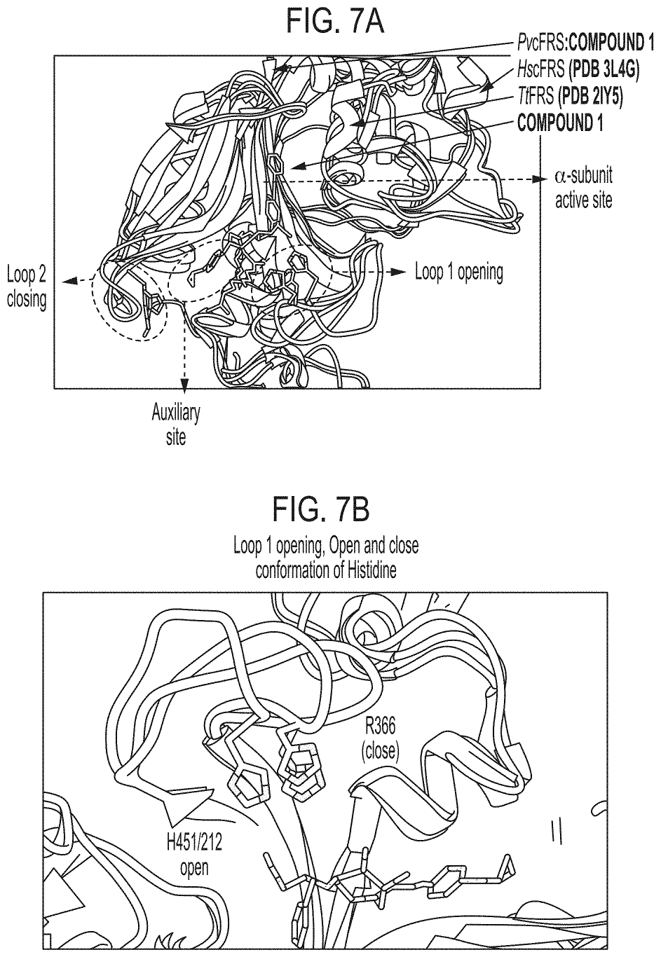

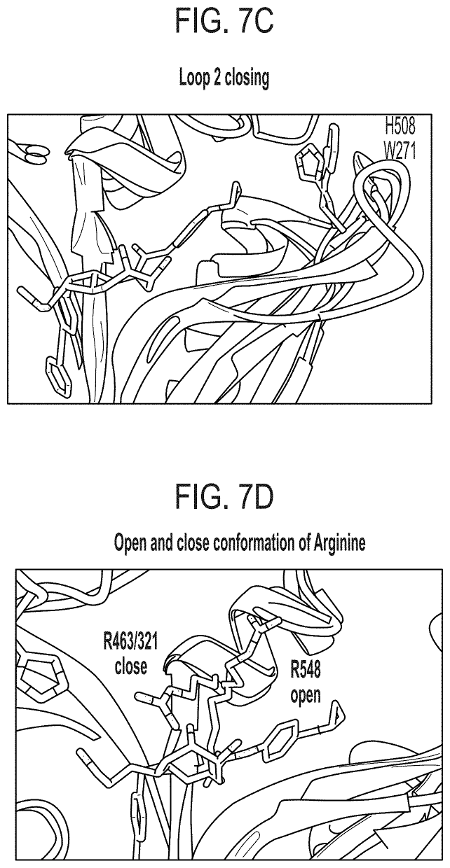

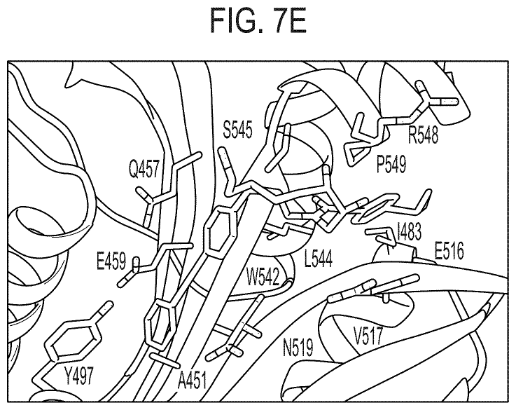

The PvcFRS: Compound 1 complex is stabilized mainly by hydrophobic interactions and hydrophilic interactions at multiple sites that contribute towards recognition of the diazabicyclodecane core, 4-cyclopropoxyphenyl and diarylacetylene appendages (FIG. 6F). In this complex, two open/close conformations are highly noticeable for residues Arg548 and His451 (FIGS. 7A-C). In particular, flexing of Arg548 opens the entry point of auxiliary pocket for 4-cyclopropoxyphenyl accommodation, underscoring the induced-fit nature of Compound 1 interaction with PvcFRS (FIG. 7D). In phenyladenylate-bound TtFRS, both the guanidium moiety of Arg321 and Phe216 (PvcFRS; Phe455) provide stacking support to the adenine ring of ATP, whereas in PvcFRS: Compound 1 the corresponding guanidinium (belonging to Arg548) is displaced away from the active site, adopting an open conformation (FIGS. 7A and 7D). In L-Phe-bound HscFRS, the corresponding Arg463 moves inward (i.e., towards the active site), adopting instead a closed conformation.

Additionally, in PvcFRS: Compound 1 there are two major loop distortions within the PA1 domain: (1) a left-hand outward displacement (open conformation) of residues 443-453 (loop 1, in ATP binding pocket), and (2) a left-hand inward movement (closed) of residues 507-515 (loop 2, in auxiliary pocket) (FIGS. 7A-C). In PvcFRS: Compound 1, loop 2 adopts a closed conformation akin to phenyladenylate-bound TtFRS and unlike L-Phe-bound HscFRS (open conformation, FIG. 7C), whereas loop 1 moves relative to both TtFRS and HscFRS to achieve an open state (FIG. 7B). The diarylacetylene moiety of Compound 1 that occupies the L-Phe site is recognized via a bed of 3-strand residues Ala541-Trp542-Gly543-Leu544 (SEQ ID NO: 34) (FIG. 7E), while its edge phenyl ring is covered by Tyr497 where it provides an aromatic T-shaped π . . . π interaction at a distance of 4.2 Å. In addition, diarylacetylene is surrounded by hydrophobic elements of Asn519, Gln457 and Glu459 (FIG. 7E). As can be seen, the urea moiety of Compound 1 is buried in a groove that is composed of Gly506, His508, Glu510, Lys512-513, Leu515, I1e552 and Pro549 (FIGS. 6D and 7C-D). The urea/groove interactions are mainly stabilized by hydrophobic contacts, particularly from Pro549 and His508 while residues Val517, I1e483 and Pro549 provide sandwich support to the cyclopropoxy moiety (FIGS. 7C-D). The urea component of Compound 1 forms hydrogen bonding interactions with main-chain N-atom of Ser545 (FIG. 7D).

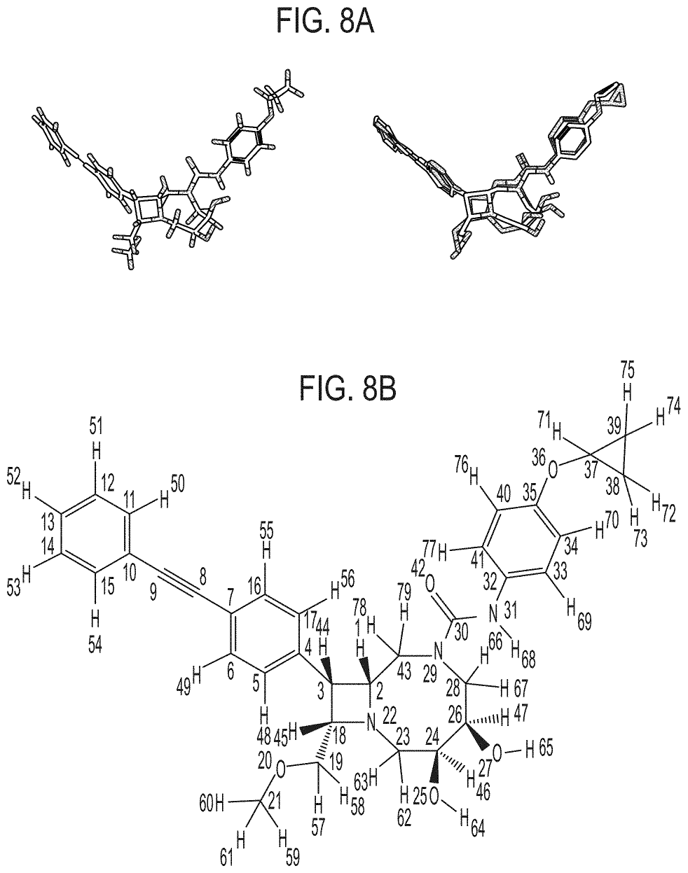

The above sets of extensive interactions position Compound 1 in an “L” shaped conformation wherein its methoxy methyl group is surrounded by socket residues Arg443, Glu445, His451 and Phe455 (not shown in figures). Interestingly, the crystallographic pose of Compound 1 is close to the conformation that the molecule is predicted to adopt in aqueous solution (FIG. 8A) based on the optimized geometry and computed energy of Compound 1, which is shown in Table 3 with each atom designation illustrated in FIG. 8B.

Table 3

| | Coordinate (Å) | | |
|---|---|---|---|
| Atom | X | Y | Z |
| H1 | 1.2953 | 3.3898 | 2.1982 |
| C2 | 0.2493 | 3.2079 | 2.4897 |
| C3 | −0.5852 | 4.5364 | 2.4896 |
| C4 | −1.6999 | 4.7921 | 1.4983 |
| C5 | −1.4906 | 5.8058 | 0.5442 |
| C6 | −2.4116 | 6.0718 | −0.4614 |
| C7 | −3.602 | 5.3236 | −0.5529 |
| C8 | −4.5235 | 5.5815 | −1.6123 |
| C9 | −5.267 | 5.8243 | −2.5446 |
| C10 | −6.1097 | 6.1215 | −3.6589 |
| C11 | −7.3337 | 5.4467 | −3.8459 |
| C12 | −8.1374 | 5.7392 | −4.9462 |
| C13 | −7.7389 | 6.7036 | −5.8761 |
| C14 | −6.5288 | 7.3806 | −5.6979 |
| C15 | −5.7185 | 7.0961 | −4.6012 |
| C16 | −3.8337 | 4.3227 | 0.4122 |
| C17 | −2.9006 | 4.0636 | 1.4148 |
| C18 | −0.7325 | 4.3255 | 4.0433 |
| C19 | −2.0842 | 4.1911 | 4.7389 |
| O20 | −2.782 | 3.0199 | 4.3425 |
| C21 | −4.0872 | 2.9597 | 4.9011 |
| N22 | 0.0691 | 3.0842 | 3.9522 |
| C23 | 1.2343 | 2.8853 | 4.7977 |
| C24 | 1.4286 | 1.4283 | 5.261 |
| O25 | 2.5187 | 1.3886 | 6.1993 |
| C26 | 1.6954 | 0.3462 | 4.1934 |
| O27 | 2.9557 | 0.6577 | 3.5812 |
| C28 | 0.5429 | 0.0876 | 3.1661 |
| N29 | 0.6024 | 0.82 | 1.8991 |
| C30 | 1.4646 | 0.5041 | 0.8712 |
| N31 | 2.0702 | −0.7388 | 0.9566 |
| C32 | 3.0404 | −1.2577 | 0.071 |
| C33 | 3.1665 | −2.6552 | −0.0175 |
| C34 | 4.1411 | −3.2358 | −0.8213 |
| C35 | 5.0092 | −2.4298 | −1.5666 |
| O36 | 5.9394 | −3.0942 | −2.3393 |
| C37 | 6.7986 | −2.3134 | −3.148 |
| C38 | 8.1225 | −2.9386 | −3.4558 |
| C39 | 8.044 | −1.734 | −2.5342 |
| C40 | 4.8899 | −1.0391 | −1.4905 |
| C41 | 3.9163 | −0.4578 | −0.6745 |
| O42 | 1.6626 | 1.2754 | −0.0832 |
| C43 | −0.2588 | 1.9973 | 1.7172 |
| H44 | 0.1238 | 5.3572 | 2.3539 |
| H45 | −0.2137 | 5.14 | 4.5729 |
| H46 | 0.5315 | 1.1268 | 5.8146 |
| H47 | 1.7963 | −0.5839 | 4.7689 |
| H48 | −0.5769 | 6.3944 | 0.587 |
| H49 | −2.2152 | 6.8549 | −1.1877 |
| H50 | −7.6443 | 4.6959 | −3.1258 |
| H51 | −9.0776 | 5.2109 | −5.08 |
| H52 | −8.3676 | 6.9269 | −6.734 |
| H53 | −6.2146 | 8.1317 | −6.4173 |
| H54 | −4.7776 | 7.621 | −4.4634 |
| H55 | −4.7507 | 3.7417 | 0.3655 |
| H56 | −3.1074 | 3.2967 | 2.1514 |
| H57 | −2.6802 | 5.0941 | 4.5204 |
| H58 | −1.92 | 4.1725 | 5.8301 |
| H59 | −4.5415 | 2.0223 | 4.5691 |
| H60 | −4.057 | 2.972 | 6.0015 |
| H61 | −4.7147 | 3.7993 | 4.5636 |
| H62 | 1.1172 | 3.4813 | 5.7103 |
| H63 | 2.1599 | 3.2347 | 4.3072 |
| H64 | 3.3302 | 1.4018 | 5.6593 |
| H65 | 3.4337 | −0.1708 | 3.4148 |
| H66 | 0.4934 | −0.99 | 2.9709 |
| H67 | −0.4066 | 0.3437 | 3.6437 |
| H68 | 1.6461 | −1.4272 | 1.5654 |
| H69 | 2.4923 | −3.292 | 0.5515 |
| H70 | 4.2344 | −4.3166 | −0.8844 |
| H71 | 6.2807 | −1.7529 | −3.9257 |
| H72 | 8.5238 | −2.8139 | −4.4577 |
| H73 | 8.3308 | −3.9029 | −2.9984 |
| H74 | 8.1996 | −1.9079 | −1.4728 |
| H75 | 8.3905 | −0.7685 | −2.8924 |
| H76 | 5.56 | −0.3978 | −2.0514 |
| H77 | 3.8373 | 0.62 | −0.6217 |
| H78 | −1.2667 | 1.739 | 2.0575 |
| H79 | −0.2945 | 2.2218 | 0.6501 |

| | |
|---|---|
| Energy (solution phase) | −1858.22081138481 Hartree |

The similarity between the bicyclic azetidine and the solution phase optimized geometry highlights the importance of the three-dimensional shape and rigidity of the diazabicyclodecane scaffold in pre-orienting the molecular appendages for an optimal target engagement. From overlaying the structure of PvcFRS: Compound 1 with that of phenyladenylate-bound TtFRS, it is apparent that the diazabicyclodecane core and its methoxymethyl extension partially brush past the adenine binding region of the canonical ATP binding site (FIG. 6C and FIG. 7B).

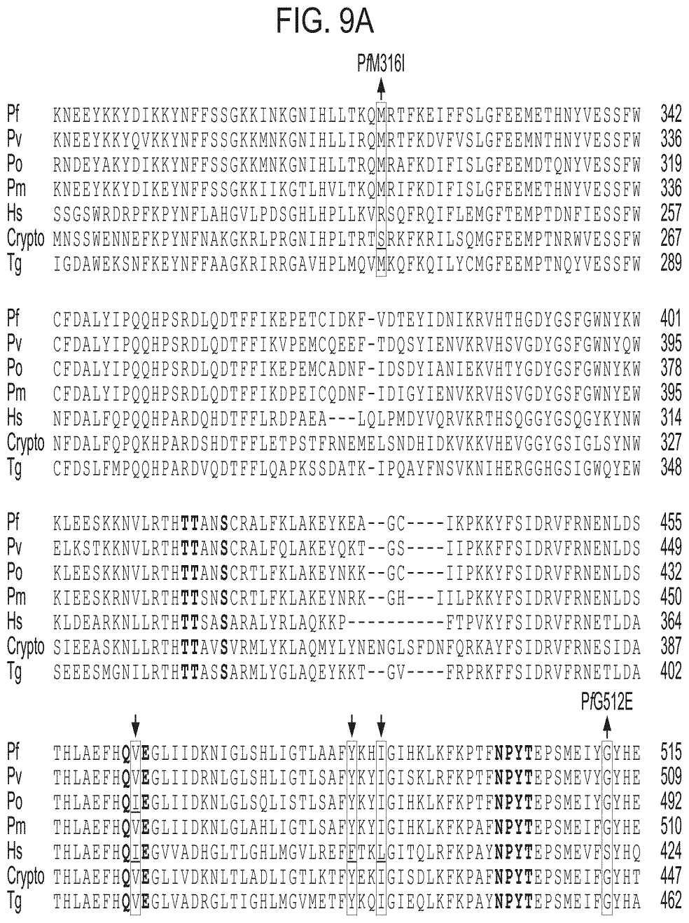

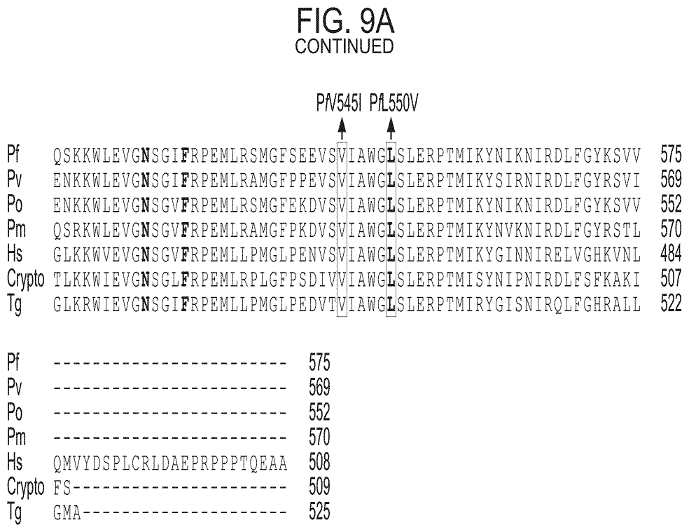

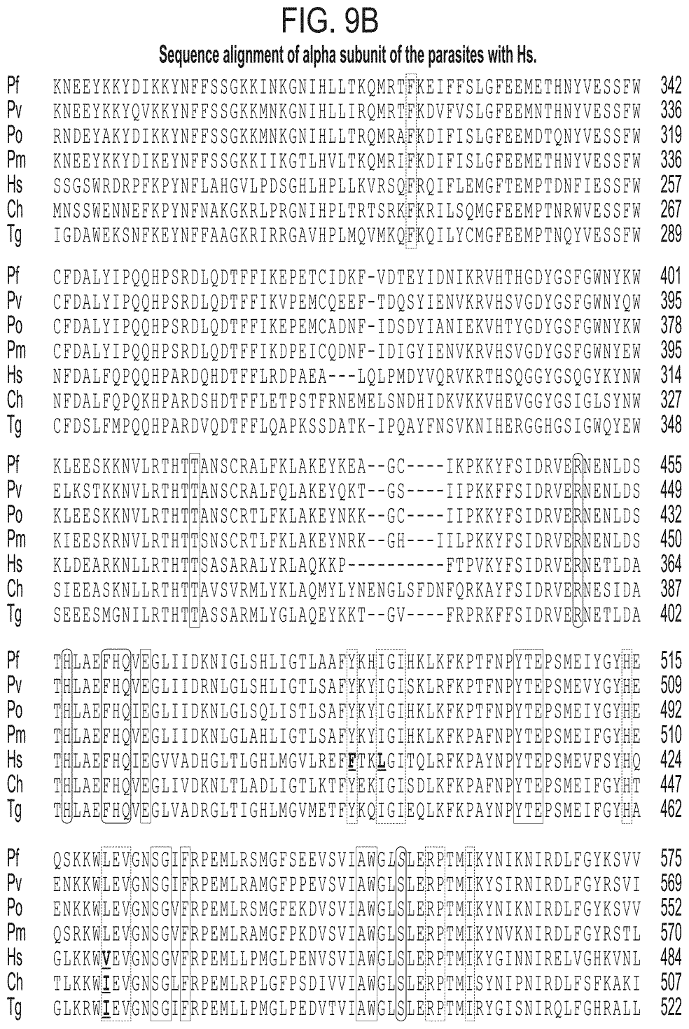

Without intending to be bound by theory, given the very high binding affinity of Compound 1 for PvcFRS (4 nM, Table 1), it is therefore possible that Compound 1 may block the interaction of Plasmodium cFRS with L-Phe first and then with ATP. Indeed, upon incubation of PvcFRS with high concentrations of both Compound 1 and an ATP analogue (the non-hydrolysable adenosine 5′-(β,γ-imido) triphosphate, i.e. AMPPNP) we observed only binding of Compound 1. This result further supports that bicyclic azetidine binding occludes ATP engagement. Strikingly, all residues in PvcFRS that recognize key ligand components (e.g., diazabicyclodecane core, 4-cyclopropoxy phenyl, methoxymethyl and diarylacetylene moieties) are conserved across the apicomplexan phyla, including human-infecting parasites such as Toxoplasma and Cryptosporidium (FIG. 9A) where residues forming L-Phe pocket residues are in bold, SNPs identified for PfcFRS are in the black box, Non-conserved residues within 5 Å, which may responsible for selectivity, are in grey box with the arrow pointing at it and underlined are the residues, which are different. Sequence alignment may also be seen in FIG. 9B, where L-Phe site residues within 5 Å radius of Compound 1 are shown in the solid boxes, ATP site residues present with 5 Å radius of Compound 1 are circled, and Auxiliary site residues within 5 Å radius of Compound 1 are shown in the dashed boxes. In FIG. 9B, non-conserved residues in the binding pockets are highlighted in bold and underline.

### Example 4: Selective Binding to Parasite Versus the Human Orthologue

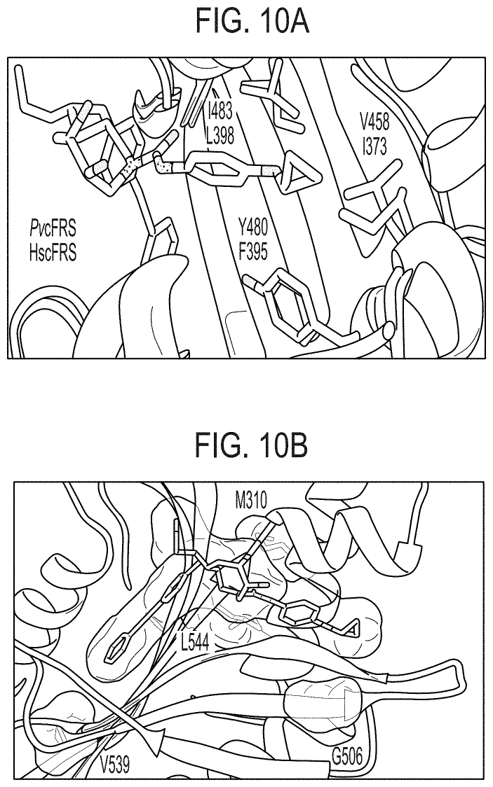

The atomic structures of PvcFRS: Compound 1 and HscFRS-L-Phe complexes were compared focusing on residues located within 5 Å of the ligand site. Three variant residues Pv-V458/Hs-1373, Pv-Y480/Hs-F395, Pv-1483/Hs-L₃₉₈ are located within an auxiliary pocket of PvcFRS (FIG. 10A). This observation indicates that the selectivity of Compound 1 arises from the terminal cyclopropyl ether (FIG. 10A). Significantly, all residues known to confer resistance to bicyclic azetidines upon mutation are located within the a subunit of FRS, in proximity to Compound 1 binding (FIG. 10B). None of these mutations, however, are directly in the ATP, tRNA or L-Phe binding sites. Table 4 shows the residues involved in PfcFRS resistance mutations and their corresponding residues in PvcFRS and HscFRS. Mutant residues are underlined.

TABLE 4

| PvcFRS | PfcFRS | HscFRS |
|---|---|---|
| M (310) | M - I (316) | R (231) |
| G (506) | G - E (512) | S (421) |
| V (539) | V - I (545) | V (454) |
| L (544) | L - V(550) | L (467) |

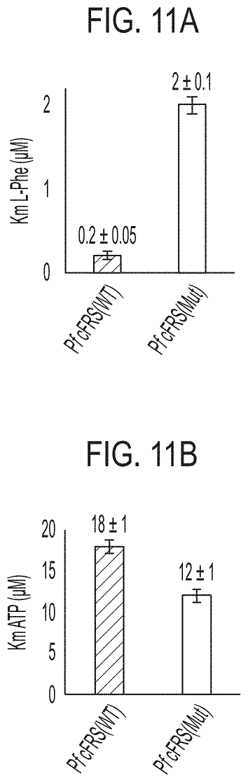

One significant mutation PvcFRS-L544V (equivalent to PfcFRS-L550V) that diminishes Compound 1 potency structurally underpins the [6.2.0]-diazabicyclodecane ring of Compound 1 (FIG. 11A). Reconstructed the PfcFRS-L550V was tested for its enzymatic fidelity and a significant increase in the Kₘ value (10-fold) was observed for the substrate L-Phe, but not for ATP (Kₘ assay performed as described in Example 2 with mutant FRS enzyme). These results indicate that resistance to bicyclic azetidines may arise from a trade-off between parasite survival and the essential nature of cFRS enzymatic activity (FIGS. 11A-B).

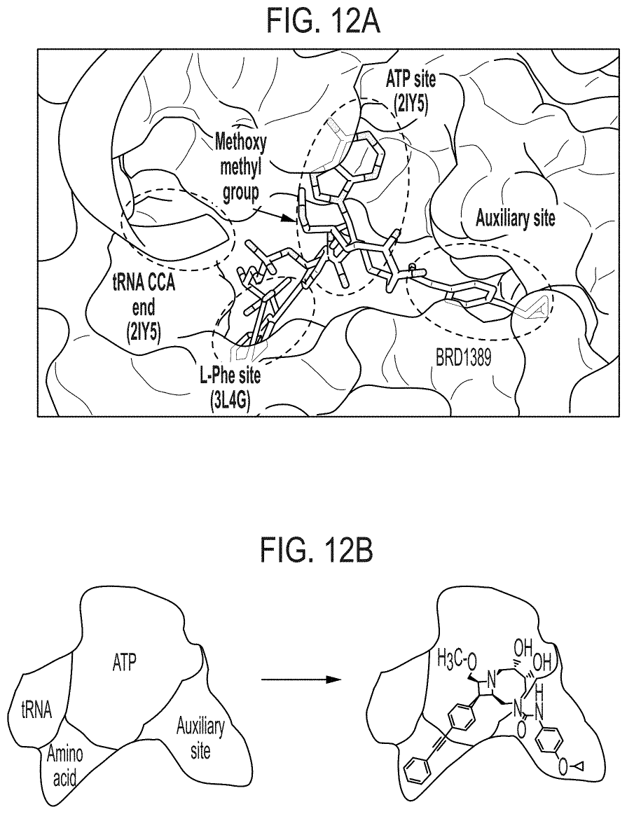

Through a combination of biochemical and crystallographic studies, the molecular underpinnings of Plasmodium cFRS inhibition by bicyclic azetidines has been elucidated by the present studies. Compound 1 was shown to inhibit parasite cFRS function by blocking the binding of both L-Phe and ATP in competitive and non-competitive fashion respectively. Specifically, the diphenylacetylene moiety of Compound 1 occupies the L-Phe binding site, while the [6.2.0]-diazabicyclodecane core partially occludes the ATP binding region. The cyclopropoxyphenyl urea region of Compound 1, in turn, occupies an auxiliary pocket in PvcFRS. Residue variations between the malaria parasite cFRS and the human orthologue in this region underpin the highly selective enzyme inhibition and parasite killing by bicyclic azetidines. Two classes of malaria parasite aaRS inhibitors have been structurally evaluated to date. These inhibitors act either as single-site occupants (cladosporin, an adenosine mimic) or dual site engagers (halofuginone, a mimic of L-Pro and 3′ end of tRNA). As certain inhibitors, such as Compound 1, occupy both the L-Phe site and an auxiliary pocket within PvcFRS, they represent novel dual-site malaria parasite aaRS inhibitors (FIGS. 12A-B).

Additionally, this mapping of protein regions and residues contributing both to cFRS inhibitor selectivity and resistance provides a structural platform for designing the next generation of compounds with improved potency and safety profiles. Indeed, the enzyme-inhibitor structure reveals how certain compound development such as the underlying principles in the diversity-oriented synthesis (DOS) library (e.g., inclusion of rigid bicyclic skeletons and multiple stereogenic elements) play a key role in accessing pockets within the enzyme that may have been inaccessible by compounds in some classical libraries. Traditional libraries are replete with compounds that have a high percentage of atoms with sp2 hybridization, leading to flatter architectures. Compound 1 and other potential inhibitors such as those having the structure of Formula (IV), in contrast make sharp turns in structure and penetrates into deep pockets within PvcFRS that are nearly at right angles.

Generation of compound libraries with tuneable drug-like properties that can focus on other apicomplexan-driven human diseases via targeting their FRSs are possible based on the present disclosure. In particular, guided by structure, triple site inhibitors can also be developed that fully occupy the ATP site via chemical modifications of the [6.2.0]-diazabicyclodecane scaffold. More generally, novel drug development against malaria and, potentially, other diseases caused by apicomplexans, such as toxoplasmosis and cryptosporidiosis are provided using the present disclosure.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof. All patents and publications mentioned in this specification are herein incorporated by reference to the same extent as if each independent patent and publication was specifically and individually indicated to be incorporated by reference.

What is claimed is:

1. A compound having the structure of formula (I): 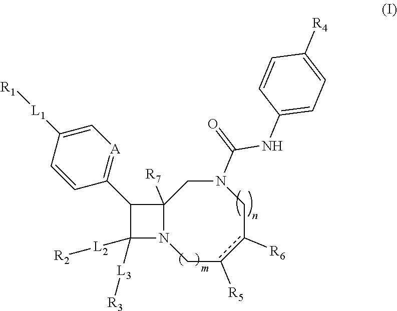
wherein the dashed bond (———) may be a single or double bond;
m is 0 or 1;
n is 0, 1 or 2;
A is CH or N;
L₁ is absent, or —C≡C—;
L₂ and L₃ are independently absent, alkylene, or heteroalkylene, wherein any of the foregoing groups optionally comprises one or more points of substitution;
R₁ is hydrogen, aryl, or heteroaryl, wherein any of the foregoing groups optionally comprises one or more points of substitution;
R₂ and R₃ are independently hydrogen, —OH, —OR, —S(O)₂R, —N(R)S(O)₂R, —C(O)R, —N(R)C(O)R, —N(R)₂, or heterocyclyl, and R₂ and/or R₃ has one or more optional points of substitution;
R₄ is cycloalkoxy optionally comprising one or more points of substitution;
R₅ and R₆ are independently selected from hydrogen and —OH;
R₇ is hydrogen, —CH₂OH, or —CH₂OR;
R is independently selected at each occurrence from hydrogen and alkyl, wherein each R has one or more optional points of substitution; and
R^{A} is independently selected at each occurrence from hydrogen and lower alkyl; or
pharmaceutically acceptable salts thereof; or
prodrugs of any of the foregoing;
wherein said compound is not

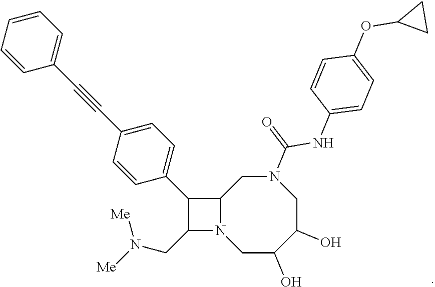 [SMILES: CN(C)CC1C(c2ccc(C#Cc3ccccc3)cc2)C2CN(C(=O)Nc3ccc(OC4CC4)cc3)CC(O)C(O)CN12]

2. The compound according to claim 1, wherein said compound has the structure of formula (II): 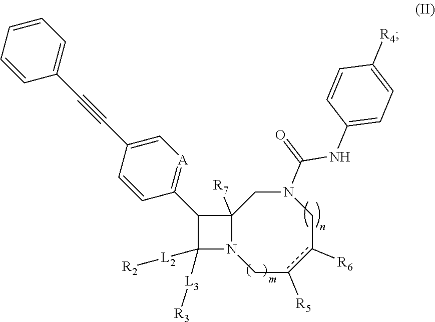
pharmaceutically acceptable salts thereof; or
prodrugs of any of the foregoing.

3. The compound according to claim 1, wherein said compound has the structure of formula (III): 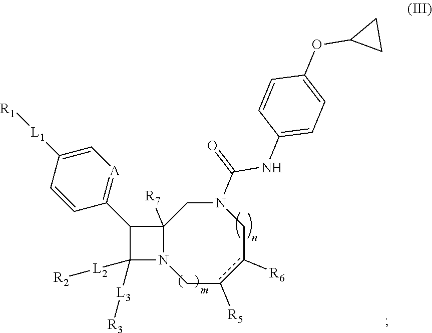
pharmaceutically acceptable salts thereof; or
prodrugs of any of the foregoing.

4. The compound according to claim 1, wherein said compound has the structure: 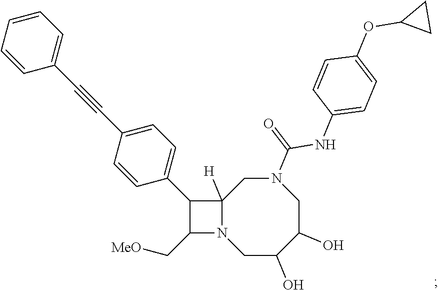 [SMILES: COCC1C(c2ccc(C#Cc3ccccc3)cc2)C2CN(C(=O)Nc3ccc(OC4CC4)cc3)CC(O)C(O)CN12]
or
an enantiomer, diastereomer, or mixture of enantiomers and/or diastereomers thereof;
or a pharmaceutically acceptable salt and/or prodrug of any of the foregoing.

5. A pharmaceutical composition comprising a pharmaceutically acceptable excipient and the compound according to claim 1, or a pharmaceutical salt thereof, or a prodrug of any of the foregoing.

6. A method of treating a parasitic disease associated with an apicomplexan parasite in a subject in need thereof comprising administering to said subject the compound according to claim 1.

7. The method of claim 6, wherein the subject is a human.

8. The method of claim 6, wherein the apicomplexan parasite is Plasmodium, Cryptosporidium or Toxoplasma.

9. The method of claim 8, wherein the Plasmodium is Plasmodium falciparum (Pj), Plasmodium vivax (Pv), Plasmodium ovale (Po), Plasmodium malariae (Pm), Plasmodium fragile (Pfr), Plasmodium inui (Pi), or Plasmodium gonderi (Pg)).

10. The method of claim 8, wherein the Cryptosporidium is Cryptosporidium parvum (Cp) or Cryptosporidium hominis (Ch).

11. The method of claim 8, wherein the Toxoplasma is Toxoplasma gondii (Tg).

12. A method of inhibiting the proliferation of an apicomplexan parasite comprising contacting said parasite with a compound according to claim 1.

13. The method of claim 12, wherein the apicomplexan parasite is Plasmodium Cryptosporidium or Toxoplasma.

14. The method of claim 13, wherein the Plasmodium is Plasmodium falciparum: (Pj), Plasmodium vivax (Pv), Plasmodium ovale (Po), Plasmodium malariae (Pm), Plasmodium fragile (Pfr), Plasmodium inui (Pi), or Plasmodium gonderi (Pg)).

15. The method of claim 13, wherein the Cryptosporidium is Cryptosporidium parvum (Cp) or Cryptosporidium hominis (Ch).

16. The method of claim 13, wherein the Toxoplasma is Toxoplasma gondii (Tg).

17. The method according to claim 14, wherein apicomplexan parasite is Plasmodium falciparum.

18. The method according to claim 14, wherein the apicomplexan parasite is Plasmodium vivax.


SEQUENCE LISTING (34 sequences)

SEQ ID NO: 1 - protein, 564 aa, Plasmodium fragile
MQAKGQEEQKGEELNHFMQVLEEEFALCHDMREKREVDDAWLGNAEAEELANHRAYYLQLQEEKNVQVEETKYVTSLHMSKKYNLEHIKVLGMAKKLETLYYVVNHVRSFNTYQLTEEGKEYLRDGSPEYVTLKYVVEQQTCSLEDLKKLFGKKGDIGLNINLKKKKIELRKSDKRLCHHVEGSANSLVDETRRHLDMVETHGHDEGTLVSHLKDIVTCGKKDEEVNNLIKDLKKRKLIEVKKVSYMYIVRTNHFTKEIKKQITDLTYLLIKNEEYKKYEVKKYNFFSSGKKMNKGNVHLLIKQMRTFKEVFISLGFEEMDTHNYVESSFWCFDALYIPQQHPSRDLQDTFFIEAPELCKDNFTDQSYIDNVKRVHSVGDYGSFGWNYPWELKSTKKNVLRTHTTANSCRALYQLAKEYQKTGSIIPKKYFSIDRVFRNENLDSTHLAEFHQVEGLIIDKNLGLSHLIGTLGAFYKYIGISKLRFKPTFNPYTEPSMEVYGYHEENKKWMEVGNSGIFRPEMLRAMGFPAEVSVIAWGLSLERPTMIKYNIRNIRDLFGYRSVL

SEQ ID NO: 2 - protein, 575 aa, Plasmodium falciparum
MSTNNVEEKKNEELYNLLNILEEEFNLSKIENKNNNKNDDGDDDDVNKREDNNNNEYYKKLKDEKGLDVLINEYTTSLKISKKYNIDHNKVMGMIKKLETLYYIINLIKSFNTYHLSDEGKQYLKEGSPEFIVIKYVIQNNKCTLEDLKKNLGKIGDIGLNTNLKNRTIQLNKADKCLTCNCNLHDIQDFTQIYLDIINQYGHDEELLFAHLKNKNHTKNALNNNEMKNSCKDNNIINNLIQDLKKRKLLEIKKNSYSHVIKTSIFKKDIKKQITDINYLLLKNEEYKKYDIKKYNFFSSGKKINKGNIHLLTKQMRTFKEIFFSLGFEEMETHNYVESSFWCFDALYIPQQHPSRDLQDTFFIKEPETCIDKFVDTEYIDNIKRVHTHGDYGSFGWNYKWKLEESKKNVLRTHTTANSCRALFKLAKEYKEAGCIKPKKYFSIDRVFRNENLDSTHLAEFHQVEGLIIDKNIGLSHLIGTLAAFYKHIGIHKLKFKPTFNPYTEPSMEIYGYHEQSKKWLEVGNSGIFRPEMLRSMGFSEEVSVIAWGLSLERPTMIKYNIKNIRDLFGYKSVV

SEQ ID NO: 3 - protein, 567 aa, Plasmodium inui
MQAKAQEEQKGEELSQFLQVLEEEFALCQDGGQKREEEEEDDPWSASTEAEELAKQRAYYLQLKEEKNVQVEEAKHVTSLHLSRKYHLEHSKVLGMAKKLETLYYVVNHVRSFNTYQLTDEGKEYLRHGSPEYVTLKYVVEQQTCTLEDLKKLFGKKGEIGLNINLREKKIQLGKSDRRLYPHVEGKADSLVDETRRYLGIVETHGHDEGTLVSHLKGILPPENQDEKVNILIRELKKRKLMEVKKVSYMYIIRTSHFRREIKKQITDLTYQLIKNEEYKKYEVKKYNFFSSGKKINKGNIHLLIKQMRTFKDVFVSLGFEEMDTHNYVESSFWCFDALYIPQQHPSRDLQDTFFVQTPELCRDNFTDKTYIDNVKRVHSVGDYGSFGWNYPWELGSTKKNVLRTHTTANSCRTLFQLAKEYQKTGSIIPKKYFSIDRVFRNENLDSTHLAEFHQVEGLIVDKNLGLSHLIGTLAAFYKYIGIFKLRFKPTFNPYTEPSMEVYGYHEENKKWMEVGNSGIFRPEMLRAMGFPPEVSVIAWGLSLERPTMIKYNIRNIRDLFGYRSVF

SEQ ID NO: 4 - protein, 552 aa, Plasmodium ovale
MNCNNGMHGNGGAEEELYSFLSLLDDEFKLCGTNDKGGSEEKREYIAYYANLKEDKGIDVMVNECTTSLHLSKKYNIEHSKVIGMIKKLETLYYVINHVKSFNTYHLTEEGMKYIRDGSPEYVVLKLVLEKKKSTMEEVKNLLGKKGEIGLNTNLKNKHIQLSKSDKSLSSHLSLHNLEDKTKKCLEIVNTYGQEEENLWKVMKNIHNGDNNEVNILIQDLKKRKLLEVKKNSYSYVVKTSKFKKDIKKQITDLNYLLLRNDEYAKYDIKKYNFFSSGKKMNKGNIHLLTRQMRAFKDIFISLGFEEMDTQNYVESSFWCFDALYIPQQHPSRDLQDTFFIKEPEMCADNFIDSDYIANIEKVHTYGDYGSFGWNYKWKLEESKKNVLRTHTTANSCRTLFKLAKEYNKKGCIIPKKYFSIDRVFRNENLDSTHLAEFHQIEGLIIDKNLGLSQLISTLSAFYKYIGIHKLKFKPTFNPYTEPSMEIYGYHEENKKWLEVGNSGVFRPEMLRSMGFEKDVSVIAWGLSLERPTMIKYNIKNIRDLFGYKSVV

SEQ ID NO: 5 - protein, 570 aa, Plasmodium malariae
MNANIVEDEQKNEELSRFIYILDKEFKLCKEEAKKEEEAKKEEEAKKDRGGEKKEEQDEYYSNLKKEKGIEVLPNEYASSLSISKKYDLDHNKVVGLMKKLEVLYYIINTVKTFNTYHLTEEGLQYLKEGTPEYLVLKYVVLEKKTCSIEDLKKKFGKKGDIGLNVNLKNRTIQLNKTDKSLSSSVDVNKLQDVTQMYLSLIDMHGHDEQLLMKEVERVGLINDVITDGHVSCVMQNLRKRKLVEVKKNSYCYVVKTSAFKKDIKKQITDLNYLLLKNEEYKKYDIKEYNFFSSGKKIIKGTLHVLTKQMRIFKDIFISLGFEEMETHNYVESSFWCFDALYIPQQHPSRDLQDTFFIKDPEICQDNFIDIGYIENVKRVHSVGDYGSFGWNYEWKIEESKRNVLRTHTTSNSCRTLFKLAKEYNRKGHIILPKKYFSIDRVFRNENLDSTHLAEFHQVEGLIIDKNLGLAHLIGTLSAFYKYIGIHKLKFKPAFNPYTEPSMEIFGYHEQSRKWLEVGNSGVFRPEMLRAMGFPKDVSVIAWGLSLERPTMIKYNVKNIRDLFGYRSTL

SEQ ID NO: 6 - protein, 495 aa, Plasmodium vivax
MVLPLPKLIQSNEGRVLYHIANHPIRTVKKKIESFFKYESLDNLNSDISVRQNFDELFVPLTHPARNIKDTFYLCKNYVKYFSTLHNDLYTRLENTNGIYKYYLTTKLVGHHKILLKRTHMTAHLPDLLRRNYKNVIYTGSVYRRDEIDRFHFPIFHQTDGFLIRPDNFDVETDLKTNLQQLIRYLFDASDIKMKWDGNTSFPFTDPSYELYIRGVPSGGRGTTRDDNNGGAASASLQGDPRGGEPPWVEVLGCGKIKKEVIAMALHAEDIRQMIEDEIARHDRGLLGELHSMEAAASGAAAEGTAPNEVTPNVATPNPATSNVAPPKGILDKLCSAPLNNRIETQMNKFLKTIRYQGWAFGIGLERLAMLLYHIHDIRLFWSTDSRFIDQFEEDVITKFHPFSNFPPVEKDISFYVSSQFKEALFFQICRDIASENIEQVKKIDSYHNPRNNQTSVCYRITYRSHSQNLTHKAVNELQSKIAQMLVKQCAVTIR

SEQ ID NO: 7 - protein, 569 aa, Plasmodium vivax
MQAKAQEEQKGEELSQFLQALEEEFALCQEGGGDKREADGPSLENAEAEELANRRAYYLQLKEERNVLVEESKHVTSLHMSSKHNIEHAKVLGMAKKLETLYYVVNHVRSFNTYQLTEEGKEYLRDGSPEHVTLRYVMEQEGCTLEDLKKLFGKKGEIGLNINLKKKKIELRKSDKRLFPHVEGSAHSLVDETRCYLQKVEAHGNDEGALVSHLKGILPPEKGEKKEEDEANNLIKELKKRKLIEAKKISYIYIIRTNLFTKEIKKQITDLTYLLIKNEEYKKYQVKKYNFFSSGKKMNKGNIHLLIRQMRTFKDVFVSLGFEEMNTHNYVESSFWCFDALYIPQQHPSRDLQDTFFIKVPEMCQEEFTDQSYIENVKRVHSVGDYGSFGWNYQWELKSTKKNVLRTHTTANSCRALFQLAKEYQKTGSIIPKKFFSIDRVFRNENLDSTHLAEFHQVEGLIIDRNLGLSHLIGTLSAFYKYIGISKLRFKPTFNPYTEPSMEVYGYHEENKKWLEVGNSGIFRPEMLRAMGFPPEVSVIAWGLSLERPTMIKYSIRNIRDLFGYRSVI

SEQ ID NO: 8 - protein, 509 aa, Cryptosporidium hominis
MEVEPVTILEKLESLNCSLSSISISEELKCEHERVIGVIKSLESRNIIKTQFISDNMLSLTDEGKDYLLNGSPEYRLIKIILEKSDHKIKQDEAMTFLGGSIFKIGLSKCLQKKLIKLNKELGLIEIPADSNMDLLNTDELGKNLFIVGVNGIKESQLTKQDLQIITELKKRKLVVSQKMSYFMINKGEDFTVKLKPMITDLTQEMIMNSSWENNEFKPYNFNAKGKRLPRGNIHPLTRTSRKFKRILSQMGFEEMPTNRWVESSFWNFDALFQPQKHPARDSHDTFFLETPSTFRNEMELSNDHIDKVKKVHEVGGYGSIGLSYNWSIEEASKNLLRTHTTAVSVRMLYKLAQMYLYNENGLSFDNFQRKAYFSIDRVFRNESIDATHLAEFHQVEGLIVDKNLTLADLIGTLKTFYEKIGISDLKFKPAFNPYTEPSMEIFGYHTTLKKWIEVGNSGLFRPEMLRPLGFPSDIVVIAWGLSLERPTMISYNIPNIRDLFSFKAKIFS

SEQ ID NO: 9 - protein, 525 aa, Toxoplasma gondii
MADVASAALMDDFLQVLNKEFGLQPVVSTLALAENSEYGGKFGAHEKIVGLVKSLQANEYVLGEQVEKTVWSLTEEGQLYAKEGSPEFRLVAWLQQRGNEEASLDVAAIKAGFGSEADVAVSNAMKMQWLRIDKATKLLHINKVPESDNVKTLLDVIQGQRDGNQDDAAALFKQLSELTATASPEKALQDLKKRKLVELRKVKYLKLQKGPKFSLHLMKPVADLTAELLIGDAWEKSNFKEYNFFAAGKRIRRGAVHPLMQVMKQFKQILYCMGFEEMPTNQYVESSFWCFDSLFMPQQHPARDVQDTFFLQAPKSSDATKIPQAYFNSVKNIHERGGHGSIGWQYEWSEEESMGNILRTHTTASSARMLYGLAQEYKKTGVFRPRKFFSIDRVFRNETLDATHLAEFHQVEGLVADRGLTIGHLMGVMETFYKQIGIEQLKFKPAYNPYTEPSMEIFGYHAGLKRWIEVGNSGIFRPEMLLPMGLPEDVTVIAWGLSLERPTMIRYGISNIRQLFGHRALLGMA

SEQ ID NO: 10 - protein, 623 aa, Plasmodium falciparum
MPTISVYEDDLFEKLGEEIIEEKLLDVCFDFGLEVDDIEYKNDKKIYKIEVPANRYDLICVEGLCRALKNFMCKFDDIKYDISMNNYDICIKGNQYIKVDGSVDDRRGYVVCCVLKNMNINDSVYNNIIEIQEKLHHNLGKKRSVLAIGIHDYDKIKFPLKYKFEKKEKINFIPLNEKTNLNGMNLIDFYSKNLNLKPYLKIIKDFDKYPIIVDSNEQILSLPPIINCDHTKISLNTKNVFIECTAIDRNKAQIALNILCSMLSEYCVPKYSIQSFVVIYENQDFSDDQNLKKKETQFLYPIFENKSLTCNIDYVRKLSGISHITVHEVNNLLKRMMLSCDIMDNNTFKVTIPFYRSDIMHCCDIIEDIAIAYGYGNIKYEPPQICKKHSLNNCSELFRNVLVECGYTEVMTNALLSRDENYNCMLRTHKSYDDPNINLDEYNPLAAPIQIKNSKTSEYEIIRTSLIVNLLKFVSANKHRELPLRFFEIGDVSYATYNQTDTNAVNKKYLSIIFSDKFTAGLEELHGVLEAILKEYQLFSDYKIEEKKKENISIRSDMYYKLIPKEDPSFLNERIVDIVLFPHNLKFGVLGIIHPKVLENFSLDIPVSAIEINVETLLNVLMM

SEQ ID NO: 11 - protein, 617 aa, Plasmodium vivax
MPTISVHEEDLIEKLGEKIEEEKLNDICFEFGIEIDDVEYKGEKKIYKIEVPANRYDLVCVEGLCRALKSFIGKYENVSYALLTNSEEACVKEKHFMRVDESVDERRSYVVSAVLKNVKMNENVYNNIIELQEKLHHNLGKKRILLAIGIHDYDKINFPVAYKFEEKEKINFIPLNETQNVNGNNFINFYQDNINLKSYLKIISDFEKFPVIVDAGGQILSLPPIINCDYTKITYDTRNLFIECTAIDRNKAEIAVNIICSMLSEYCTPKYSIHSFFVQYDKNHKAEKGNGYLYPVFKNKTLTCHMDYVRKLSGILNLSVKDVEPLLKKMMITSKVIDSSTFTVDVPFYRSDIMHFCDIVEDIAIAYGYGNIVSEKIEIAKKNSLSACTELFRNVLAECTYTEVMTNALLSKRENYDCMLRKHRSYDDRKINLDEYNPLAPPVQIMNSKTSEYEIVRTSLIVNMLKFVSANKHRELPLRFFEIGDVSYTTYDRTDTNAVNKRYLSVIFADKFTAGLEEAHGMLETVLKEFQLFSDYKIEEKSKENVAIRSDVFYKLVPKEDPSFLNERVVDIVLCPHNLKFGIMGIIHPKVLENFSIDIPVSVIEINIETIMDVLMM

SEQ ID NO: 12 - protein, 617 aa, Plasmodium fragile
MPTISVYEEDLIEKLGQKIEEEKLNDICFEFGIEIDDIEYKGEKKIYKIEVPANRYDLVCVEGLCRALKSFIGKCQNINYVLLQNSEEACVKEKHFIRVDESVDERRSYVVSAVLKNVKINENVYNNIIELQEKLHHNLGKKRILLAIGIHDYDKIKFPVVYKFEEKDKINFIPLNETKNVNGNNFLKFYQDNINLKSYLKIIKDFEKFPVIVDADNKILSLPPIINCDHTKITYDTKNLFIECTAIVKNKAEIAVNIICSMLSEYCTPKYSIHSFFVQYDKKHKAEKGNGYLYPIFKNKSLTCHMDYVRKLSGILDLSVKDVEPLLKKMMISSKIIDSNTFTVDVPFYRSDIMHCCDIVEDIAIAYGYGNIVSEKIEIAKKNSLSACTELFRNVLVECTYTEVMTNALLSKRENYDCMLRKHRDYKDEKINLDEYNPLAPPVQIMNSKTSEYEIVRTSLIVNMLKFVSANKHRELPLRFFEIGDVSYTTYNKTDTNAVNKRYLSVIFADKFTAGLEEAHGMLETVLKEFQLFSDYKIEEKRKENVAIRSDVFYKLVPTKDPSFLNERVVDIILCPQNLKFGILGIIHPKVLENFSIDVPVSVMEINIESIMNILMI

SEQ ID NO: 13 - protein, 617 aa, Plasmodium malariae
MPTISVFEEDLIEKLEERLSDEKLQDICFDFGLEVDDIEYKNKKKIYKIEVPANRYDLVCVEGLCRALKSFIGKFDNIKYDILMNSHECCIKEKHYINVKESVDERRSYVVSCVLKNIKMTEHVYNNIIELQEKLHHNIGKKRTVLAIGIHDYDKIKFPVEYKFEEKSKINFIPLNETKNLNGNNLMEFYEHNTNLKPYLKIIKNFEKYPLIVDANNQILSLPPIINCEYTKITLNTKNLFVECTATDKHKAEIALNIICSMLSEYCTPKYSIYSFLVLYDQNHKIEKGNSYLYPEFKNKVVTCEIDYVRKLSGIPNITVEDVEKVLKKMMIPIKIIDNCTFTAHVPFYRSDIMHSCDIVEDIAIGYGYGNIKSCEVEFSKKHLLNTCSDLFRNALIECAYTEVMTNALLSKRENYNCMLRKYKDYKDVQINLEEYNPLAPPVQIMNSKTSEYEIIRTSLIVNLLKFVASNKHRELPLRFFEIGDVSYTTYNKTDTNAVNKRHLSVIFADKFCSGLEEIHGVLETVLKEFQLFNDYKIDEKKKENISLRSDVFYKLLPKEDSSFLSERVVDIVLFPHNLKFGVLGIIHPEVLGNFSIDIPVSAVEINIETLLNVLMM

SEQ ID NO: 14 - protein, 573 aa, Plasmodium gonderi
MNKKEKGNQNVEKNENTFEEQKEDEMNHFLKVLEEEFGLCREKYEKKEDVQMNEFGIDVASHRSYYLQLKDEKNIEVLENEHTTSLCVSEKHNLEHSKVLGMVKKLETLYYVINHVKSFNKYQLTEEGEEYLKSGSPEYVTLKFVVENEKCTLDHLKKMFGKKGEIGLNINLKNKKIELNKNDKSLIPRVSTTGCDIVDDTRKLLNVIKAEGHDEHMLFTHLKETLFSKLDSDVNNLIANLKKRKLMEVKKVSYSYIIRTSRFEKKIKKQITDMNYQLMKNEEYKKYDIKKYNFFSSGKKINKGNIHLLTKQMRTFKEIFISLGFEEMETHNYVESSFWCFDALYIPQQHPSRDLQDTFFIKIPELCNENFIDSEYIENVKRVHSVGDYGSFGWNYNWELRESKKNVLRTHTTANSCRALFKLAKEYKKTGSIIPKKYFSIDRVFRNENLDSTHLAEFHQVEGLIIDKNLGLSHLIATLASFYKYIGIHKLRFKPTFNPYTEPSMEVYGYHEQNKKWIEVGNSGIFRPEMLQAMGFSDEVSVIAWGLSLERPTMIKYNIRNIRDLFGYRSLIA

SEQ ID NO: 15 - protein, 623 aa, Plasmodium falciparum; identical to SEQ ID NO: 10

SEQ ID NO: 16 - protein, 509 aa, Cryptosporidium hominis; identical to SEQ ID NO: 8

SEQ ID NO: 17 - protein, 647 aa, Cryptosporidium hominis
MPVVSVSTRVLSSYLNKEVTSEWLDEVCFNFGLELDCIEFDEEIKDNVAKIEIPANRPDILCLEGLVIALGCFIGNSNIPLFNLKPKGDDQKIIVKRNVARVRPFILCAILRDLSFNEDIYRSFIDYQEKLHNNICRKRSLVSIGTHDLDMIEGPFYYDAKAHSEIKFVPLIGSAEVDGNQLLDLLNKHQQLKKYVSLVENEIFLPVVTDSNGIVLSVPPLINGSQSKITLNTKNVFIEVTATDYNRASIVLNQIVSSFSMYCKNKFEIEPVKVEYEHSTYPPFPHKYEVLANGNYHVVTPNVERLEFVVKASEASNLLGINPILDPIVAQSLLRKMMIDSEIIKDNESLKCFVPINRSDILHPVDIIEDIGISFGFNNIELKKSRFYELDKSNLMAEQVKRELSLLGISESLNWALCKHSDCFESLFRTENIKLKNLFESEVQYNLNCPPVVVKDAKTSEFEILRTTLIQSLLKTIASNKSLPLPQKIFEVGDVVILDNNTPSGSRNDKRVAIAYCNSSGSGLEEIHGFLDQLLSKLGLVAEYSLNEPNSIHPNIIGMYSLSEVNDPSFLPTRSVKITIQKVELNSCLKSIDIEKTMLNKQVVIGIMGVVHPKVLNNFSLTLPTSVLELRLEPIMHLWPDTFFYDE

SEQ ID NO: 18 - protein, 675 aa, Toxoplasma gondii
MPTVSVPRDELFRRLGRTYSVHEFEELCFEFGIELDEVVEPGKDGSTETIYKIEVPANRYDLLCTEGISRALYAFNNPDAPLPAYRLEPATPQFTMTVKPAVNQVRPFVVCAILRNVTLTKAGLASFIEFQDKLHHTLCRRRSLVAIGTHDLSKIQPPFVYDARPPKNFEFVPLGCDSQMNGEQVMAHFSSHLQLKAYLPLIQNSPVYPLILDAKDRILSLPPIINSEFSKVTEDTRDIFIECTAVDITKAQIVLNTLVAMFSEYCKEPYTVEPIRVVYEDPSSAPIDRSVKCQGEASLQNGSASMNGWVFPRVNSRSMPFSLDYVRQLTGIPDLTADACANLLKRMMIHTSIEKATQAGILEASIPITRSDILHERDIVEDVAIAYSFNRLPVTRSYMLTGDALNCLSEKIRNFCTVCGYTEALNFSLSSAAENSSSLGRTPGDGKSSLFNPLEYQVNAQPVRLANSKTREFDQVRTTLISGLLKTIVANKGRRELPIKLFEIGDVCLLDSTTEVGARNLRYCCLAFADEHSSGLEEVHGVLDALLQSLQFVGEYAIAEMEAAVAAAAAAEKAGCEHASAHATEQLQRTLSRIRGTFKLVPSHEETFLPGRQVQVVASLKSSGDMENVPVLLGVMGTLHPHTLKAFGLTIPVSIFELNVEAFVQWLPAIDLVVG

SEQ ID NO: 19 - protein, 508 aa, Homo sapiens
MADGQVAELLLRRLEASDGGLDSAELAAELGMEHQAVVGAVKSLQALGEVIEAELRSTKHWELTAEGEEIAREGSHEARVFRSIPPEGLAQSELMRLPSGKVGFSKAMSNKWIRVDKSAADGPRVFRVVDSMEDEVQRRLQLVRGGQAEKLGEKERSELRKRKLLAEVTLKTYWVSKGSAFSTSISKQETELSPEMISSGSWRDRPFKPYNFLAHGVLPDSGHLHPLLKVRSQFRQIFLEMGFTEMPTDNFIESSFWNFDALFQPQQHPARDQHDTFFLRDPAEALQLPMDYVQRVKRTHSQGGYGSQGYKYNWKLDEARKNLLRTHTTSASARALYRLAQKKPFTPVKYFSIDRVFRNETLDATHLAEFHQIEGVVADHGLTLGHLMGVLREFFTKLGITQLRFKPAYNPYTEPSMEVFSYHQGLKKWVEVGNSGVFRPEMLLPMGLPENVSVIAWGLSLERPTMIKYGINNIRELVGHKVNLQMVYDSPLCRLDAEPRPPPTQEAA

SEQ ID NO: 20 - protein, 589 aa, Homo sapiens
MPTVSVKRDLLFQALGRTYTDEEFDELCFEFGLELDEITSEKEIISKEQGNVKAAGASDVVLYKIDVPANRYDLLCLEGLVRGLQVFKERIKAPVYKRVMPDGKIQKLIITEETAKIRPFAVAAVLRNIKFTKDRYDSFIELQEKLHQNICRKRALVAIGTHDLDTLSGPFTYTAKRPSDIKFKPLNKTKEYTACELMNIYKTDNHLKHYLHIIENKPLYPVIYDSNGVVLSMPPIINGDHSRITVNTRNIFIECTGTDFTKAKIVLDIIVTMFSEYCENQFTVEAAEVVFPNGKSHTFPELAYRKEMVRADLINKKVGIRETPENLAKLLTRMYLKSEVIGDGNQIEIEIPPTRADIIHACDIVEDAAIAYGYNNIQMTLPKTYTIANQFPLNKLTELLRHDMAAAGFTEALTFALCSQEDIADKLGVDISATKAVHISNPKTAEFQVARTTLLPGLLKTIAANRKMPLPLKLFEISDIVIKDSNTDVGAKNYRHLCAVYYNKNPGFEIIHGLLDRIMQLLDVPPGEDKGGYVIKASEGPAFFPGRCAEIFARGQSVGKLGVLHPDVITKFELTMPCSSLEINVGPFL

SEQ ID NO: 21 - protein, 57 aa, Plasmodium vivax
DSSTFTVDVPFYRSDIMHFCDIVEDIAIAYGYGNIVSEKIEIAKKNSLSACTELFRN

SEQ ID NO: 22 - protein, 57 aa, Plasmodium falciparum
DNNTFKVTIPFYRSDIMHCCDIIEDIAIAYGYGNIKYEPPQICKKHSLNNCSELFRN

SEQ ID NO: 23 - protein, 60 aa, Homo sapiens
DGNQIEIEIPPTRADIIHACDIVEDAAIAYGYNNIQMTLPKTYTIANQFPLNKLTELLRH

SEQ ID NO: 24 - protein, 293 aa, Plasmodium falciparum
KNEEYKKYDIKKYNFFSSGKKINKGNIHLLTKQMRTFKEIFFSLGFEEMETHNYVESSFWCFDALYIPQQHPSRDLQDTFFIKEPETCIDKFVDTEYIDNIKRVHTHGDYGSFGWNYKWKLEESKKNVLRTHTTANSCRALFKLAKEYKEAGCIKPKKYFSIDRVFRNENLDSTHLAEFHQVEGLIIDKNIGLSHLIGTLAAFYKHIGIHKLKFKPTFNPYTEPSMEIYGYHEQSKKWLEVGNSGIFRPEMLRSMGFSEEVSVIAWGLSLERPTMIKYNIKNIRDLFGYKSVV

SEQ ID NO: 25 - protein, 293 aa, Plasmodium vivax
KNEEYKKYQVKKYNFFSSGKKMNKGNIHLLIRQMRTFKDVFVSLGFEEMNTHNYVESSFWCFDALYIPQQHPSRDLQDTFFIKVPEMCQEEFTDQSYIENVKRVHSVGDYGSFGWNYQWELKSTKKNVLRTHTTANSCRALFQLAKEYQKTGSIIPKKFFSIDRVFRNENLDSTHLAEFHQVEGLIIDRNLGLSHLIGTLSAFYKYIGISKLRFKPTFNPYTEPSMEVYGYHEENKKWLEVGNSGIFRPEMLRAMGFPPEVSVIAWGLSLERPTMIKYSIRNIRDLFGYRSVI

SEQ ID NO: 26 - protein, 293 aa, Plasmodium ovale
RNDEYAKYDIKKYNFFSSGKKMNKGNIHLLTRQMRAFKDIFISLGFEEMDTQNYVESSFWCFDALYIPQQHPSRDLQDTFFIKEPEMCADNFIDSDYIANIEKVHTYGDYGSFGWNYKWKLEESKKNVLRTHTTANSCRTLFKLAKEYNKKGCIIPKKYFSIDRVFRNENLDSTHLAEFHQIEGLIIDKNLGLSQLISTLSAFYKYIGIHKLKFKPTFNPYTEPSMEIYGYHEENKKWLEVGNSGVFRPEMLRSMGFEKDVSVIAWGLSLERPTMIKYNIKNIRDLFGYKSVV

SEQ ID NO: 27 - protein, 294 aa, Plasmodium malariae
KNEEYKKYDIKEYNFFSSGKKIIKGTLHVLTKQMRIFKDIFISLGFEEMETHNYVESSFWCFDALYIPQQHPSRDLQDTFFIKDPEICQDNFIDIGYIENVKRVHSVGDYGSFGWNYEWKIEESKRNVLRTHTTSNSCRTLFKLAKEYNRKGHIILPKKYFSIDRVFRNENLDSTHLAEFHQVEGLIIDKNLGLAHLIGTLSAFYKYIGIHKLKFKPAFNPYTEPSMEIFGYHEQSRKWLEVGNSGVFRPEMLRAMGFPKDVSVIAWGLSLERPTMIKYNVKNIRDLFGYRSTL

SEQ ID NO: 28 - protein, 311 aa, Homo sapiens
SSGSWRDRPFKPYNFLAHGVLPDSGHLHPLLKVRSQFRQIFLEMGFTEMPTDNFIESSFWNFDALFQPQQHPARDQHDTFFLRDPAEALQLPMDYVQRVKRTHSQGGYGSQGYKYNWKLDEARKNLLRTHTTSASARALYRLAQKKPFTPVKYFSIDRVFRNETLDATHLAEFHQIEGVVADHGLTLGHLMGVLREFFTKLGITQLRFKPAYNPYTEPSMEVFSYHQGLKKWVEVGNSGVFRPEMLLPMGLPENVSVIAWGLSLERPTMIKYGINNIRELVGHKVNLQMVYDSPLCRLDAEPRPPPTQEAA

SEQ ID NO: 29 - protein, 302 aa, Cryptosporidium hominis
MNSSWENNEFKPYNFNAKGKRLPRGNIHPLTRTSRKFKRILSQMGFEEMPTNRWVESSFWNFDALFQPQKHPARDSHDTFFLETPSTFRNEMELSNDHIDKVKKVHEVGGYGSIGLSYNWSIEEASKNLLRTHTTAVSVRMLYKLAQMYLYNENGLSFDNFQRKAYFSIDRVFRNESIDATHLAEFHQVEGLIVDKNLTLADLIGTLKTFYEKIGISDLKFKPAFNPYTEPSMEIFGYHTTLKKWIEVGNSGLFRPEMLRPLGFPSDIVVIAWGLSLERPTMISYNIPNIRDLFSFKAKIFS

SEQ ID NO: 30 - protein, 296 aa, Toxoplasma gondii
IGDAWEKSNFKEYNFFAAGKRIRRGAVHPLMQVMKQFKQILYCMGFEEMPTNQYVESSFWCFDSLFMPQQHPARDVQDTFFLQAPKSSDATKIPQAYFNSVKNIHERGGHGSIGWQYEWSEEESMGNILRTHTTASSARMLYGLAQEYKKTGVFRPRKFFSIDRVFRNETLDATHLAEFHQVEGLVADRGLTIGHLMGVMETFYKQIGIEQLKFKPAYNPYTEPSMEIFGYHAGLKRWIEVGNSGIFRPEMLLPMGLPEDVTVIAWGLSLERPTMIRYGISNIRQLFGHRALLGMA

SEQ ID NO: 31 - protein, 287 aa, Homo sapiens
SSGSWRDRPFKPYNFLAHGVLPDSGHLHPLLKVRSQFRQIFLEMGFTEMPTDNFIESSFWNFDALFQPQQHPARDQHDTFFLRDPAEALQLPMDYVQRVKRTHSQGGYGSQGYKYNWKLDEARKNLLRTHTTSASARALYRLAQKKPFTPVKYFSIDRVFRNETLDATHLAEFHQIEGVVADHGLTLGHLMGVLREFFTKLGITQLRFKPAYNPYTEPSMEVFSYHQGLKKWVEVGNSGVFRPEMLLPMGLPENVSVIAWGLSLERPTMIKYGINNIRELVGHKVNL

SEQ ID NO: 32 - protein, 300 aa, Cryptosporidium hominis
MNSSWENNEFKPYNFNAKGKRLPRGNIHPLTRTSRKFKRILSQMGFEEMPTNRWVESSFWNFDALFQPQKHPARDSHDTFFLETPSTFRNEMELSNDHIDKVKKVHEVGGYGSIGLSYNWSIEEASKNLLRTHTTAVSVRMLYKLAQMYLYNENGLSFDNFQRKAYFSIDRVFRNESIDATHLAEFHQVEGLIVDKNLTLADLIGTLKTFYEKIGISDLKFKPAFNPYTEPSMEIFGYHTTLKKWIEVGNSGLFRPEMLRPLGFPSDIVVIAWGLSLERPTMISYNIPNIRDLFSFKAKI

SEQ ID NO: 33 - protein, 293 aa, Toxoplasma gondii
IGDAWEKSNFKEYNFFAAGKRIRRGAVHPLMQVMKQFKQILYCMGFEEMPTNQYVESSFWCFDSLFMPQQHPARDVQDTFFLQAPKSSDATKIPQAYFNSVKNIHERGGHGSIGWQYEWSEEESMGNILRTHTTASSARMLYGLAQEYKKTGVFRPRKFFSIDRVFRNETLDATHLAEFHQVEGLVADRGLTIGHLMGVMETFYKQIGIEQLKFKPAYNPYTEPSMEIFGYHAGLKRWIEVGNSGIFRPEMLLPMGLPEDVTVIAWGLSLERPTMIRYGISNIRQLFGHRALL

SEQ ID NO: 34 - protein, 4 aa, unidentified
  REGION 1..4: note = Description of Unknown: beta-strand peptide
AWGL